(19) United States
(12) Patent Application Publication
(10) Pub. No.: US 2007/0292540 A1
(43) Pub. Date: Dec. 20, 2007

(54) Extracts and Methods Comprising Cinnamon Species

(76) Inventors: Robert T. Gow, Naples, FL (US); Dan Li, Singapore (SG); George W. Sypert, Naples, FL (US); Randall S. Alberte, Estero, FL (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/690,627

(22) Filed: Mar. 23, 2007

Related U.S. Application Data
(60) Provisional application No. 60/785,012, filed on Mar. 23, 2006.
(60) Provisional application No. 60/873,475, filed on Dec. 7, 2006.

(51) Int. Cl. A61K 36/54; A23L 1/28; A61P 39/06; C07C 29/00; C07C 39/12; C07C 39/18; C07C 47/54; C07C 63/04; C07D 311/04; C07H 17/02

(52) U.S. Cl. 424/739; 426/655; 536/123.1; 536/8; 549/397; 549/403; 549/406; 562/405; 568/425; 568/716; 568/717; 568/840; 585/22

(57) ABSTRACT

The present invention relates to extracts of cinnamon species plant material prepared by supercritical CO₂ extractions methods.

### RELATED APPLICATIONS

This application claims the benefit of priority to U.S. Provisional Patent Applications Ser. Nos. 60/785,012, filed Mar. 23, 2006, and 60/873,475, filed Dec. 7, 2006, which are hereby incorporated by reference in their entirety.

### FIELD OF INVENTION

The disclosure relates in part to extractions derived from cinnamon species, having an elevated essential oil amount, an elevated phenolic acid amount, an elevated proanthocyanidin amount, and/or an elevated polysaccharide amount, methods of preparing such extractions, and methods for use of such extractions.

### BACKGROUND OF THE INVENTION

Cinnamon (Cinnamomum zeylanicum or verum, C. aromaticum, and C. cassia) is a small evergreen tree 10-15 meters tall that is native to tropical southern India and Sri Lanka and grows from sea level to elevations of nine hundred meters. It has thick scabrous bark and strong branches. Young shoots are speckled greenish orange. The leaves are petiolate and leathery when mature, with a shiny green upper side and lighter underside. The leaves smell spicy and have a hot taste. The fruit is an oval berry, larger than a blackberry; like an acorn in its receptacle. The fruit is bluish when ripe with white spots on it, with a taste like Juniper and a terebine smell. When boiled, it gives off an oily matter which is called cinnamon suet. The root-bark smells like cinnamon and tastes like camphor, which can be isolated via distillation. “cinnamon”, the medicinal part of cinnamonum species, consists of the dried bark, separated from the cork and the underlying parenchyma, of young branches and shoots of Cinnamoum species.

Cinnamon species were introduced throughout the islands of the Indian Ocean and Southeast Asia, and are now cultivated extensively in Sri Lanka and the coastal regions of India. Sri Lanka is the main producing country, though substantial cinnamon product comes from India, Malaysia, Madagascar and the, Seychelles. Cinnamon bark has been used in traditional Eastern and Western medicines for several thousand years. According to the energetics theory in traditional Chinese medicine (TCM), cinnamon acts to supplement the body fire, to warm and tone the spleen and kidney; thus making it effective for chest and abdominal pain, diarrhea due to asthenia, and hypofunction of the kidney. Galenical preparations of cinnamon are used as a carminative, digestive, or stomachic component of compounds in TCM, traditional Greco-European medicines, and traditional Indian Ayurvedic and Unani medicine. The German Commission E approved the internal use of cinnamon for loss of appetite and dyspeptic complaints such as mild spasms of the gastrointestinal tract, bloating, and flatulence. In the United States and Germany, cinnamon is used as a carminative and stomachic component of herbal compounds in dosage forms including aqueous infusion or decoction, alcoholic fluid extract or tincture, and essential oil. It also appears as a component of multi-herb cough, cold, and fever formulas. More recently, scientific evidence has supported the use of cinnamon for type 2 diabetes (NIDDM-non-insulin dependent diabetes mellitus), anti-oxidant activity, anti-platelet adhesive activity, anti-inflammatory activity, anti-bacterial and fungal activity, and enhancement of brain function. See Khan A et al. Diabetes Care 26:3215-3218, 2003; Anderson R A et al. J Agric Food Chem 52:65-70, 2004; Jarville-Taylor et al. J Am Coll Nutri 20:327-336, 2001; Qin R et al. Horm Metab Res 36:119-123, 2004; Vespohl E J et al. Phytother Res 19:203-206, 2005; Lee S H et al Biochem Pharmacol 69:791-9, 2005; Chericoni S et al. J Agric Food Chem 53:4762-4765, 2005; Lin C C et al. Phytother Res 17:7260730, 2003; Jayaprakasha G K et al. J Agric Food Chem 51:4344-4348, 2003; Huss U et al. J Nat Prod 65:1517-21, 2002; Nagai H et al. Jpn J Pharmacol 32:813-822, 1982; Su M J et al. J Biomed Sci 6:376-386, 1999; Shimada Y et al. Phytomed 11:404-410, 2004; Taher M et al. Med J Malayia 59B:97-98, 2004; Kamath J V et al. Phytother Res 17:970-972, 2003; Kurokawa M et al. Eur J Pharmacol 348:45-51, 1998; Simic A et al. Phytother Res 18:713-717, 2004; Tabak M et al. J Ethnopharmacol 67:269-277, 1999; Kong L D et al. J Ethnopharmacol 73:199-207, 2000; Kwon B M et al. Arch Pharm Res 21:147-152, 1998; Ka H et al. Cancer Lett 196:143-152, 2003.

The chemical constituents of cinnamon bark include the essential oils (volatile and non-volatile), polyphenolic acids, coumarin, gum, muscilage, resin, carbohydrates (starch, polysaccharides), and ash (Table 1). From a commercial and biological standpoint, the essential oil (particularly the cinnamaldehydes and terpenes) and the polyphenolic acids (particularly the flavonol glycosides-proanthocyanidins and flavonoids) have been traditionally considered to be of greater importance than the other constituents. Polyphenolic compounds contain more than one hydroxyl group (OH) on one or more aromatic rings. The physical and chemical properties, analysis, and biological activities of polyphenols and particularly flavonoids have been studied for many years. However, other chemical constituents such as the polysaccharides may also have important biologically beneficial effects. Like all botanicals, the chemical composition of cinnamon bark varies with species, age of harvest, climate, soil, and horticultural practices.

TABLE 1
Principal Chemical Constituents of Cinnamon Bark

| | % dry |
|---|---|
| Chemical constituents | weight |
| Essential Oils | 1-4% |
| Volatile Oils | |
| Trans-cinnamaldehyde | (60-80%) |
| Benzaldehyde | |
| 2′-hydroxycinnamaldehyde | |
| 2-methoxycinnamaldehyde | |
| 2′-benzoxycinnamaldehyde | |
| Eugenol | (up to 10%) |
| Trans-cinnamic acid | (5-10%) |
| Cinnamyl acetate | |
| Cinnamyl alcohol | |
| Linalool | |
| 1,8-cineole | |
| Monoterpenes and Sesquiterpenes | (1-3%) |
| Alpha-Pinene | |
| Beta-pinene | |
| Borneol | |
| Polyphenols | 5-10% |
| Flavonol glycosides | |
| Kaempferitrin | |
| Kaempferol 3-O-Beta-D-glucopyranosyl-(1→4)-alpha- | |
| L-rhamnopyranoside | |
| Kaempferol 3-O-beta-D-apiofuranosyl-(1→2)-alpha- | |
| L-rhamnopyranoside | |
| Kaempferol 3-O-beta-D-apiofuranosyl-(1→4)-alpha- | |
| L-rhamnopyranoside | |
| Flavonoids | |
| Methylhydroxychalcone | |
| catechin | |
| epicatechin | |
| anthocyanidin | |
| Catechin/Epicatechin oligomers | |
| 3-(2-hydroxyphenyl)-propanoic acid | |
| 3-(2-hydroxyphenyl)-O-glycoside | |
| Proanthocyanidins | |
| Condensed Tannins | |
| Calcium-monterpenes oxalate | |
| Gum | |
| Muscilage | |
| Resin | |
| Carbohydrates | 80-90% |
| Starch | |
| Polysaccharides | |
| Ash | |

### SUMMARY OF THE INVENTION

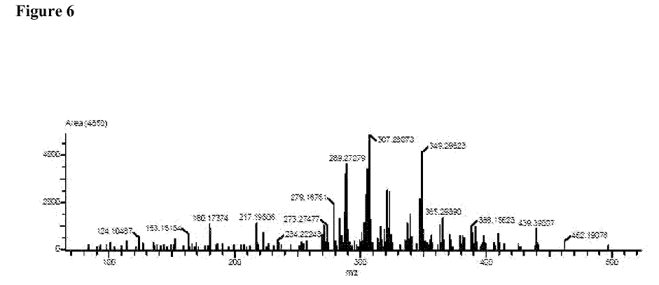

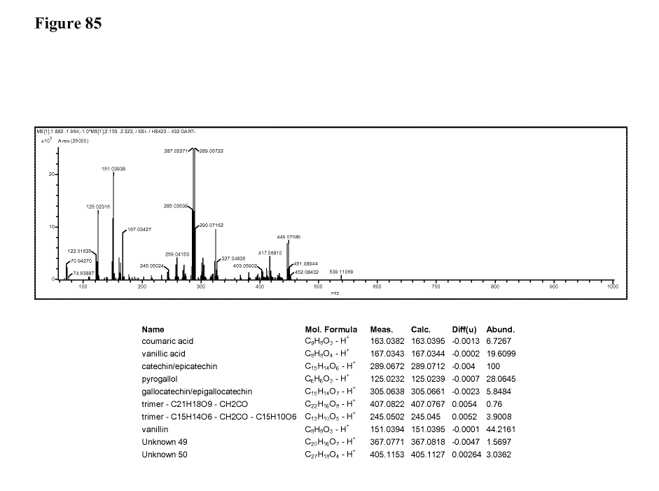

In one aspect, the present invention relates to a cinnamon species extract comprising a fraction having a Direct Analysis in Real Time (DART) mass spectrometry chromatogram of any of FIGS. 6 to 85.

In a further embodiment, the fraction comprises a compound selected from the group consisting of cinnamaldehyde, benzaldehyde, cinnamyl alcohol, trans-cinnamic acid, cinnamyl acetate, an essential oil, a polyphenol, a polysaccharide, and combinations thereof.

In a further embodiment, the fraction comprises cinnamaldehyde in an amount greater than about 2% by weight. In a further embodiment, the fraction comprises cinnamaldehyde in an amount greater than about 5, 10, 15, 20, 25, 30, 35, 40, 45, 50, 55, 60, 65, 70, 75, 80, 85, 90, or 95% by weight. In a further embodiment, the fraction comprises cinnamaldehyde in an amount from about 65% to about 95% by weight.

In a further embodiment, the fraction comprises an essential oil selected from the group consisting of eugenol, 2′-hydroxycinnamaldehyde, 2-methoxycinnamaldehyde, 2′-benzoxycinnamaldehyde, linalool, 1,8-cineole, alpha-pinene, beta-pinene, and combinations thereof. In a further embodiment, the fraction comprises essential oil in an amount from about 1% to about 5% by weight. In a further embodiment, the fraction comprises a combined amount of cinnamaldehyde and essential oil of about 5% to about 40% by weight.

In a further embodiment, the fraction comprises a polyphenol selected from the group consisting of flavonoid, flavonol glycoside, and combinations thereof. In a further embodiment, the flavonoid is selected from the group consisting of 3-(2-hydroxyphenyl)-propanoic acid, 3-(2-hydroxyphenyl)-O-glycoside, anthocyanidin, epitcatechin, catechin, methylhydroxychalcone, catechin oligomers, epicatechin oligomers, oligomeric proanthocyanidins, polymeric proanthocyanidins, and combinations thereof. In a further embodiment, the flavonol glycoside is selected from the group consisting of kaempferitrin, kaempferol 3-O-Beta-D-glucopyranosyl-(1→4)-alpha-L-rhamnopyranoside, kaempferol 3-O-beta-D-apiofuranosyl-(1→2)-alpha-L-rhamnopyranoside, kaempferol 3-O-beta-D-apiofuranosyl-(1→4)-alpha-L-rhamnopyranoside, and combinations thereof. In a further embodiment, the fraction comprises a polyphenol in an amount from about 20% to about 70% by weight. In a further embodiment, the fraction comprises cinnamaldehyde at about 6% by weight and a polyphenol at about 70% by weight. In a further embodiment, the fraction comprises cinnamaldehyde at about 40% by weight and a polyphenol at about 20% by weight.

In a further embodiment, the fraction comprises a polysaccharide selected from the group consisting of glucose, arabinose, galactose, rhamnose, xylose uronic acid and combinations thereof. In a further embodiment, the fraction comprises a polysaccharide at about 30% by weight.

In another aspect, the present invention relates to a food or medicament comprising the cinnamon species extract of the present invention.

In another aspect, the present invention relates to a method for making a cinnamon extract comprising sequentially extracting a cinnamon species plant material to yield an essential oil fraction, a non-tannin polyphenolic fraction and a polysaccharide fraction by a) extracting cinnamon species plant material by supercritical carbon dioxide extraction to yield the essential oil fraction and a first residue; b) extracting cinnamon species plant material or the first residue from step a) with hot water to yield the polysaccharide fraction and a second residue; and c) extracting cinnamon species plant material, the first residue from step a) and/or the second residue from step b) with a hydro-alcoholic solution and purifying the extraction using affinity adsorbent processes to yield the non-tannin polyphenolic fraction.

In a further embodiment, step a) comprises 1) loading in an extraction vessel ground cinnamon species plant material; 2) adding carbon dioxide under supercritical conditions; 3) contacting the ground cinnamon bark and the carbon dioxide for a time; and 4) collecting an essential oil fraction in a collection vessel. In a further embodiment, supercritical conditions comprise 60 bars to 800 bars of pressure at 35° C. to 90° C. In a further embodiment, supercritical conditions comprise 60 bars to 500 bars of pressure at 40° C. to 80° C. In a further embodiment, the time is 30 minutes to 2.5 hours. In a further embodiment, the time is 1 hour. In a further embodiment, a supercritical carbon dioxide fractional separation system is used for fractionation, purification, and profiling of the essential oil fraction.

In a further embodiment, step b) comprises 1) contacting ground cinnamon species plant material or the first residue from step a) with a water solution for a time sufficient to extract polysaccharide chemical constituent; and 2) separating and purifying the solid polysaccharides from the solution by alcohol precipitation. In a further embodiment, the water solution is at 80° C. to 100° C. In a further embodiment, the water solution is at 80° C. to 90° C. In a further embodiment, the time is 1-5 hours. In a further embodiment, the time is 2-4 hours. In a further embodiment, the time is 2 hours. In a further embodiment, the alcohol is ethanol.

In a further embodiment, step c) comprises: 1) contacting cinnamon species plant material, the first residue from step a) and/or the second residue from step b) with hydroalcoholic solution for a time sufficient to extract polyphenolic chemical constituents; 2) passing a concentrated alcohol solution of extracted polyphenolic chemical constituents from the hydroalcoholic solvent mixture through an affinity adsorbent resin column wherein the polyphenolic acids are adsorbed; and 3) eluting the purified non-tannin polyphenolic chemical constituent fraction(s) from the affinity adsorbent resin leaving the tannin polyphenolics adsorbed to the affinity adsorbent resin.

In a further embodiment, the hydroalcoholic solution comprises ethanol and water wherein the ethanol concentration is 10-95% by weight. In a further embodiment, the hydroalcoholic solution comprises ethanol and water wherein the ethanol concentration is 25% by weight. In a further embodiment, step 1) is carried out at 30° C. to 100° C. In a further embodiment, step 1) is carried out at 60° C. to 100° C. In a further embodiment, the time is 1-10 hours. In a further embodiment, the time is 1-5 hours. In a further embodiment, the time is 2 hours.

In another aspect the present invention relates to a cinnamon species extract prepared by the methods of the present invention.

In another aspect the present invention relates to a cinnamon species extract comprising cinnamaldehyde, cinnamic acid at 1 to 5% by weight of the cinnamaldehyde, methyl cinnamic acid at 5 to 15% by weight of the cinnamaldehyde, cinnamyl alcohol at 1 to 5% by weight of the cinnamaldehyde, β-gualenen/cis-γ-bisababolene at 20 to 30% by weight of the cinnamaldehyde, and pyrogallol at 1 to 5% by weight of the cinnamaldehyde.

In another aspect the present invention relates to a cinnamon species extract comprising pyrogallol, cinnamic acid at 80 to 90% by weight of the pyrogallol, methyl cinnamic acid at 85 to 95% by weight of the pyrogallol, coumaric acid at 20 to 30% by weight of the pyrogallol, homovanillic acid at 15 to 25% by weight of the pyrogallol, cinnamaldehyde at 85 to 95% by weight of the pyrogallol, and benzyl benzoate at 10 to 15% by weight of the pyrogallol.

In another aspect the present invention relates to a cinnamon species extract comprising catechin, cinnamic acid at 5 to 15% by weight of the catechin, methyl cinnamic acid at 5 to 15% by weight of the catechin, coumaric acid at 5 to 15% by weight of the catechin, ferulic acid at 1 to 10% by weight of the catechin, 2-methoxyphenol at 1 to 5% by weight of the catechin, homovanillic acid at 5 to 15% by weight of the catechin, vanillic acid at 20 to 30% by weight of the catechin, benzaldehyde at 1 to 5% by weight of the catechin, cinnamaldehyde at 35 to 45% by weight of the catechin, pyrogallol at 85 to 95% by weight of the catechin, and caffeic acid at to 15% by weight of the catechin.

In another aspect the present invention relates to a cinnamon species extract comprising β-gualenen/cis-γ-bisababolene and cinnamaldehyde at 5 to 15% by weight of the β-gualenen/cis-γ-bisababolene.

In another aspect the present invention relates to a cinnamon species extract comprising cinnamaldehyde and β-gualenen/cis-γ-bisababolene at 10 to 20% by weight of cinnamaldehyde.

In another aspect the present invention relates to a cinnamon species extract comprising cinnamaldehyde, pyrogallol at 30 to 40% by weight of the cinnamaldehyde, and catechin/epicatechin at 1 to 10% by weight of cinnamaldehyde.

In another aspect the present invention relates to a cinnamon species extract comprising cinnamaldehyde, cinnamic acid at 1 to 5% by weight of the cinnamaldehyde, methoxy cinnamaldehyde at 0.5 to 5% by weight of the cinnamaldehyde, eugenol at 0.1 to 5% by weight of the cinnamaldehyde, p-cymene at 1 to 5% by weight of the cinnamaldehyde, camphor at 0.1 to 5% by weight of the cinnamaldehyde, carvacrol at 0.5 to 5% by weight of the cinnamaldehyde, caryophyllene/humulene at 25 to 35% by weight of the cinnamaldehyde, pyrogallol at 0.1 to 5% of the cinnamaldehyde, and cinnamyl cinnamate at 40 to 50% by weight of the cinnamaldehyde.

In another aspect the present invention relates to a cinnamon species extract comprising cinnamyl cinnamate, methoxy cinnamaldehyde at 0.5 to 5% by weight of the cinnamyl cinnamate, cinnamyl alcohol at 0.1 to 5% by weight of the cinnamyl cinnamate, p-cymene at 1 to 5% by weight of the cinnamyl cinnamate, linalool at 0.1 to 5% by weight of the cinnamyl cinnamate, camphor at 0.1 to 5% by weight of the cinnamyl cinnamate, carvacrol at 0.5 to 5% by weight of the cinnamyl cinnamate, cinnamaldehyde at 70 to 80% by weight of the cinnamyl cinnamate, caryophyllene/humulene at 45 to 55% by weight of the cinnamyl cinnamate, and pyrogallol at 0.1 to 5% of the cinnamyl cinnamate.

In another aspect the present invention relates to a cinnamon species extract comprising pyrogallol, cinnamic acid at 5 to 10% by weight of the pyrogallol, coumaric acid at 60 to 70% by weight of the pyrogallol, ferulic acid at 1 to 10% of the pyrogallol, 2-methoxyphenol at 5 to 15% of the pyrogallol, vanillic acid at 1 to 10% by weight of the pyrogallol, catechin/epicatechin at 30 to 40% by weight of the pyrogallol, benzaldehyde at 1 to 5% by weight of the pyrogallol, afzelechin/epiafzelechin at 5 to 15% by weight of the pyrogallol, resveratrol at 1 to 10% by weight of the pyrogallol, and vanillin at 1 to 5% by weight of the pyrogallol.

In another aspect the present invention relates to a cinnamon species extract comprising pyrogallol, cinnamic acid at 0.5 to 5% by weight of the pyrogallol, coumaric acid at 10 to 20% by weight of the pyrogallol, ferulic acid at 0.5 to 5% of the pyrogallol, 2-methoxyphenol at 1 to 5% of the pyrogallol, homo/isovanillic acid at 0.5 to 5% by weight of the pyrogallol, vanillic acid at 1 to 10% by weight of the pyrogallol, catechin/epicatechin at 25 to 35% by weight of the pyrogallol, benzaldehyde at 1 to 5% by weight of the pyrogallol, cinnamaldehyde at 1 to 5% of the pyrogallol, afzelechin/epiafzelechin at 0.1 to 5% by weight of the pyrogallol, and vanillin at 65 to 75% by weight of the pyrogallol.

The extractions of the disclosure are useful in providing physiological and medical effects including, but not limited to, anti-oxidant activity, oxygen free radical scavenging, nitrosation inhibition, anti-mutagenic activity (cancer prevention), anti-carcinogenic activity (cancer therapy), skin protection, anti-aging, anti-cardiovascular disease, anti-stroke disease and therapy, cerebral protection, anti-hyperlipidemia, anti-periodontal disease, anti-osteoporosis, immunological enhancement, anti-viral, anti-HIV and anti-bacterial activity, anti-fungal activity, anti-viral activity, weight control and thermogenesis, anti-diabetes, and anxiety reduction, mood enhancement and cognitive enhancement

These embodiments of the disclosure, other embodiments, and their features and characteristics, will be apparent from the description, drawings and claims that follow.

### BRIEF DESCRIPTION OF THE INVENTION

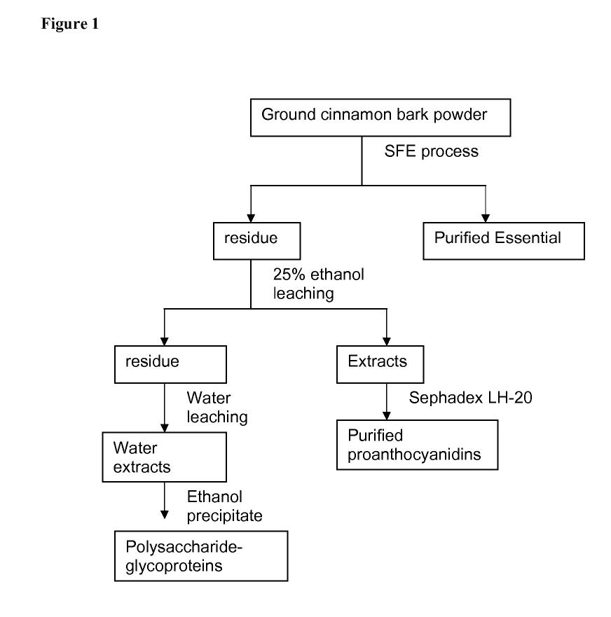

FIG. 1 depicts an exemplary schematic diagram of cinnamon extraction processes

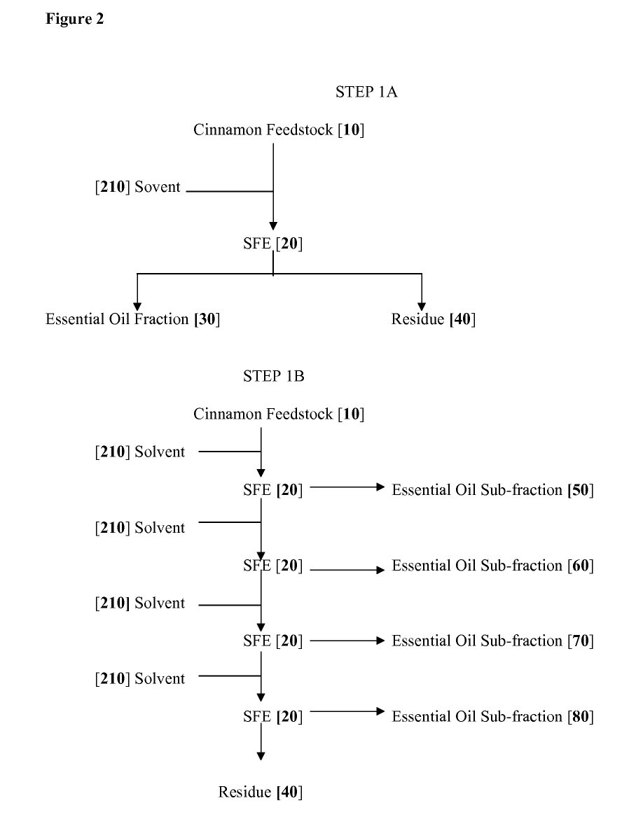

FIG. 2 depicts an exemplary method for the preparation of essential oil fractions.

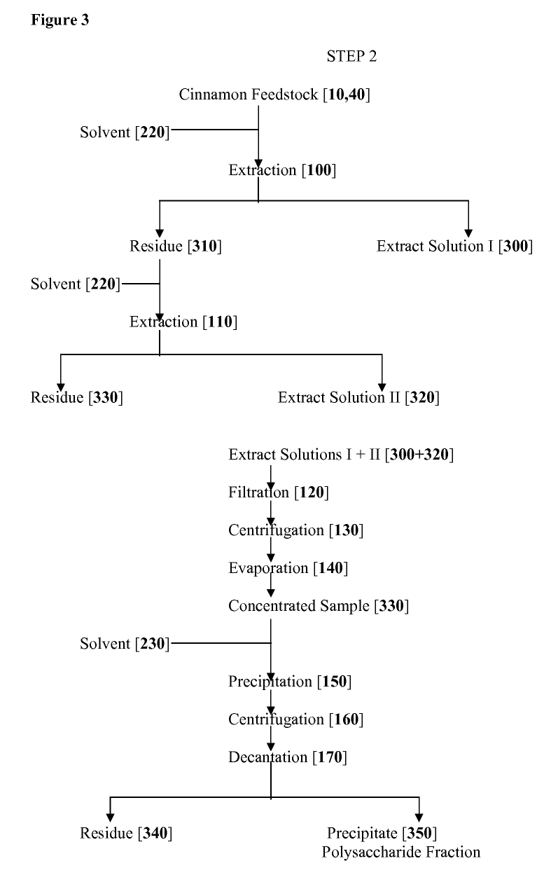

FIG. 3 depicts an exemplary method for preparation of polysaccharide fractions.

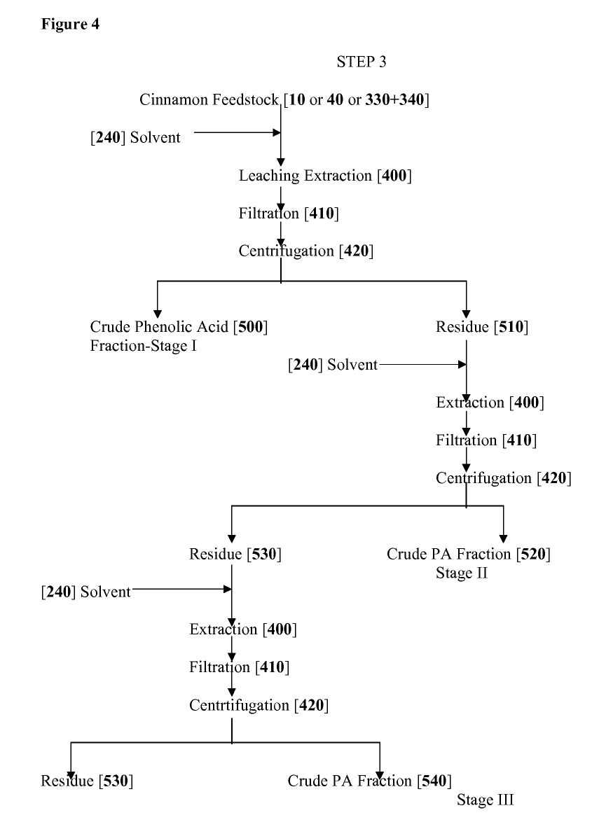

FIG. 4 depicts an exemplary method for solvent leaching extraction.

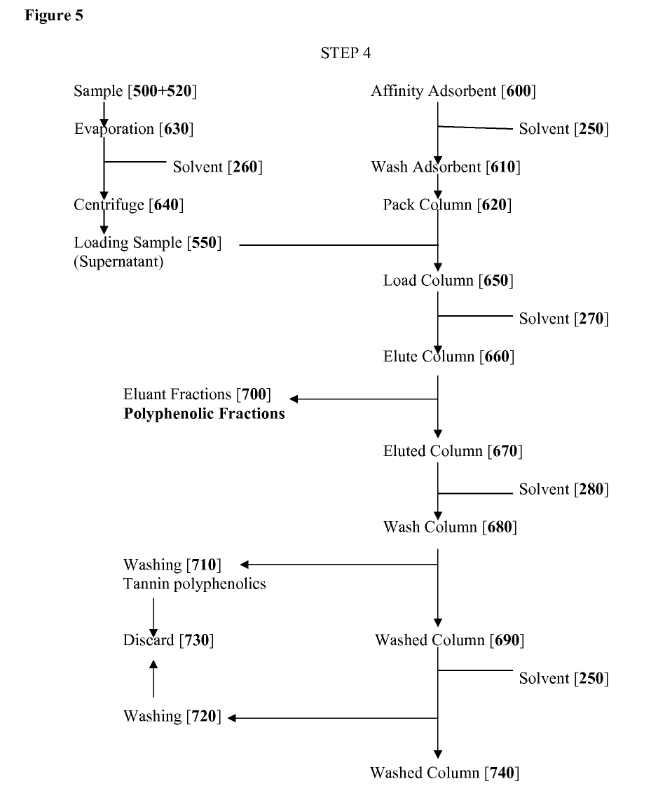

FIG. 5 depicts an exemplary method for preparation of purified polyphenolic fractions.

FIG. 6 depicts AccuTOF-DART Mass Spectrum for cinnamon polysaccharide (positive ion mode).

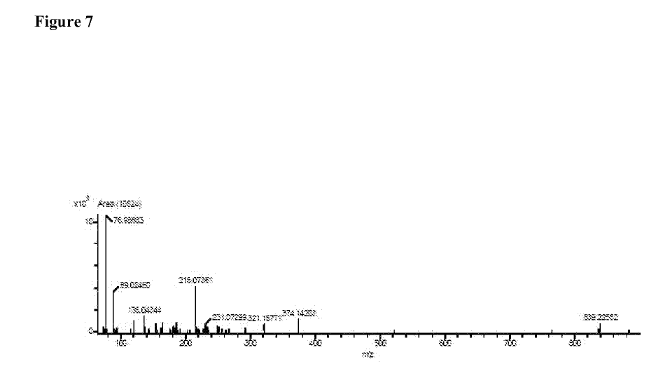

FIG. 7 depicts AccuTOF-DART Mass Spectrum for cinnamon polysaccharide (negative ion mode).

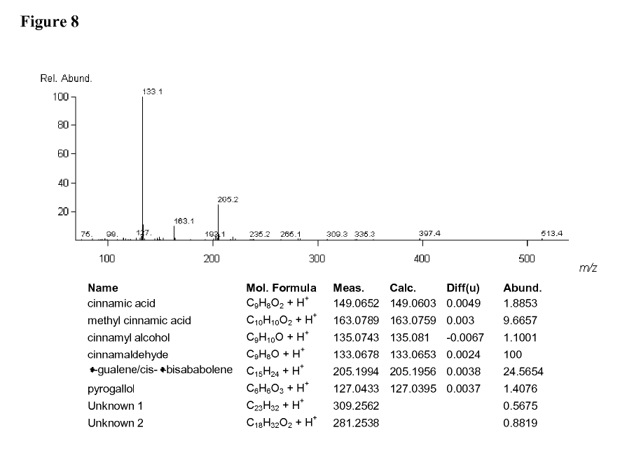

FIG. 8 depicts AccuTOF-DART Mass Spectrum for cinnamon bark (positive ion mode).

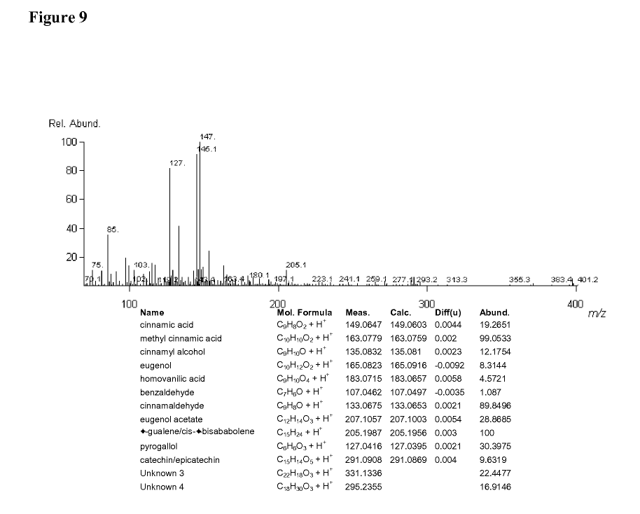

FIG. 9 depicts AccuTOF-DART Mass Spectrum for crude extract of cinnamon bark separated by column chromatography using Sephadex LH-20 packing material (positive ion mode).

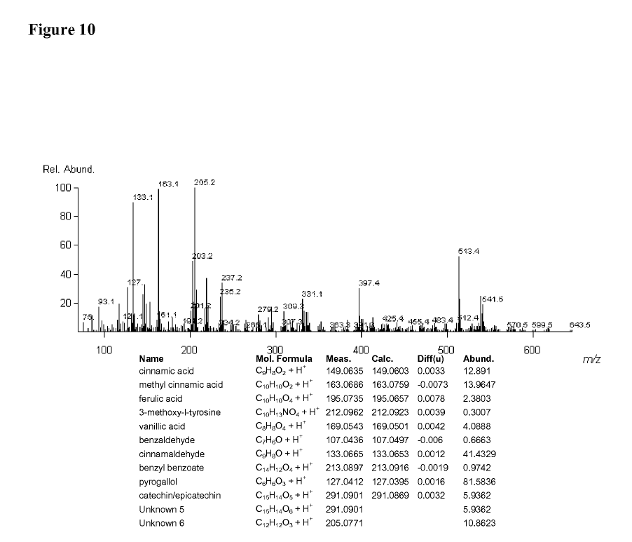

FIG. 10 depicts AccuTOF-DART Mass Spectrum for crude extract of cinnamon bark HS#147 using a 75% EtOH extraction solvent (positive ion mode).

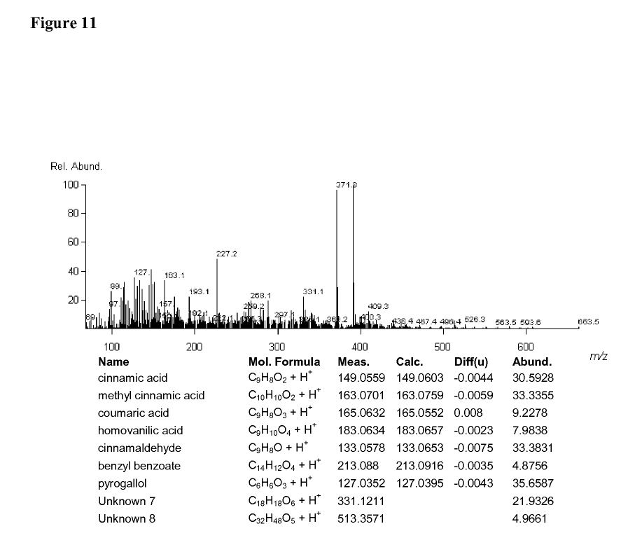

FIG. 11 depicts AccuTOF-DART Mass Spectrum for fraction F3 separated by column chromatography using Sephadex LH-20 packing material (positive ion mode).

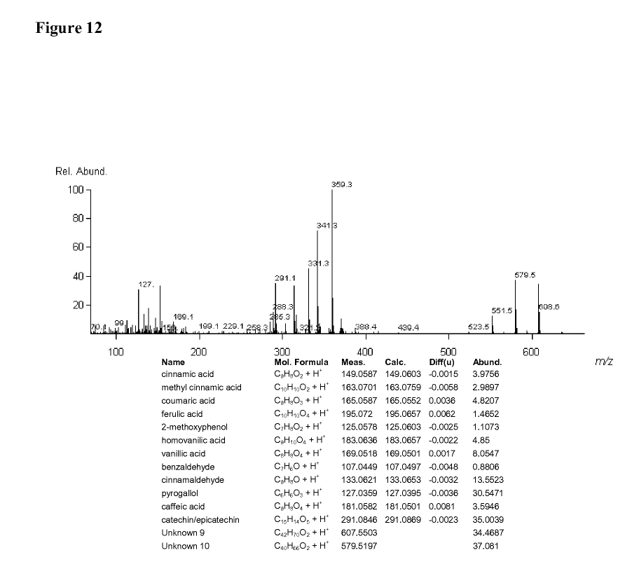

FIG. 12 depicts AccuTOF-DART Mass Spectrum for fraction F4 by column chromatography using Sephadex LH-20 packing material (positive ion mode).

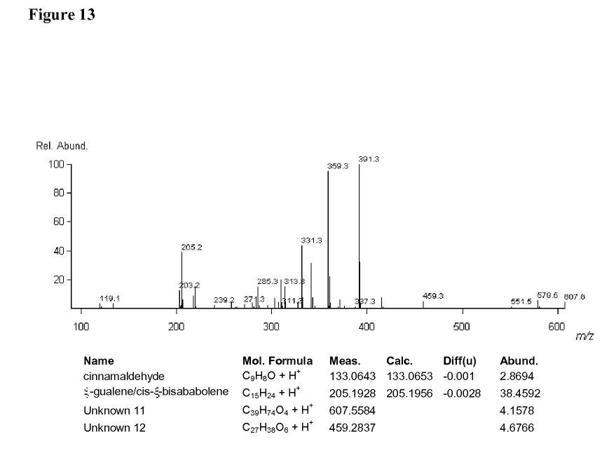

FIG. 13 depicts AccuTOF-DART Mass Spectrum for fraction F5 by column chromatography using Sephadex LH-20 packing material (positive ion mode).

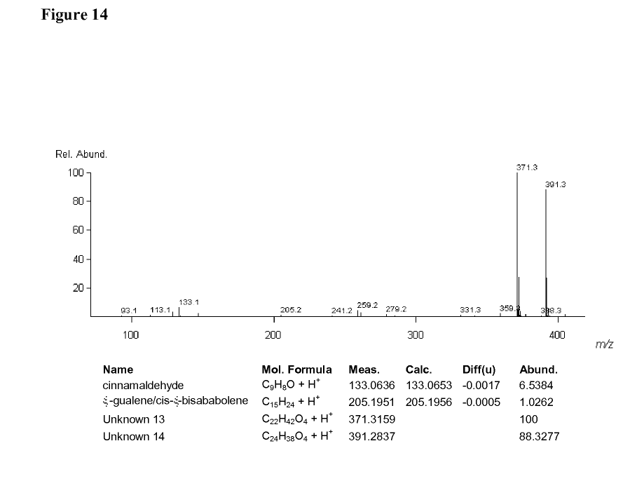

FIG. 14 depicts AccuTOF-DART Mass Spectrum for fraction F6 by column chromatography using Sephadex LH-20 packing material (positive ion mode).

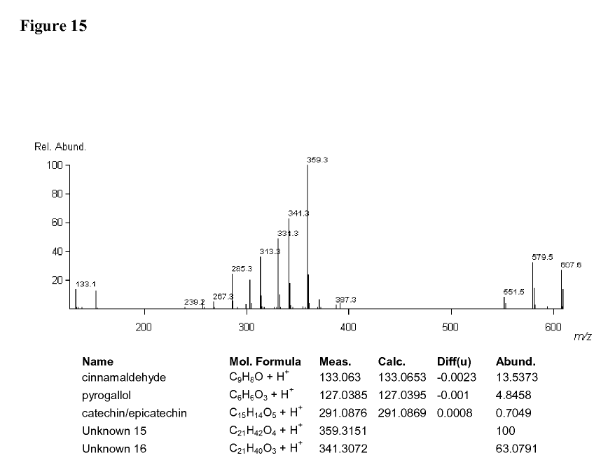

FIG. 15 depicts AccuTOF-DART Mass Spectrum for fraction F7 by column chromatography using Sephadex LH-20 packing material (positive ion mode).

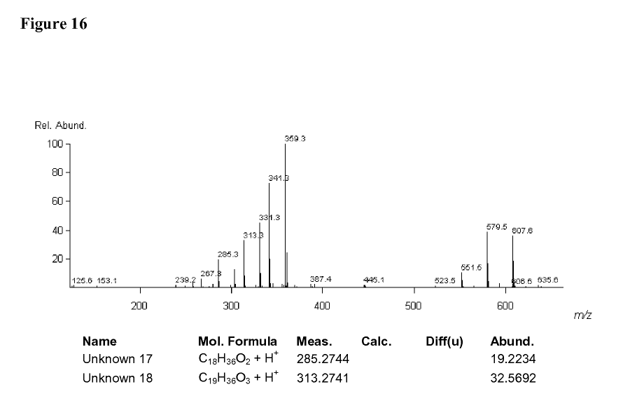

FIG. 16 depicts AccuTOF-DART Mass Spectrum for fraction F8 by column chromatography using Sephadex LH-20 packing material (positive ion mode).

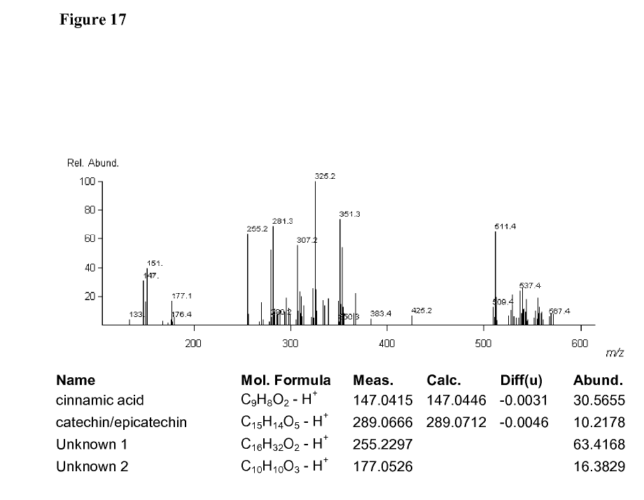

FIG. 17 depicts AccuTOF-DART Mass Spectrum for cinnamon bark (negative ion mode).

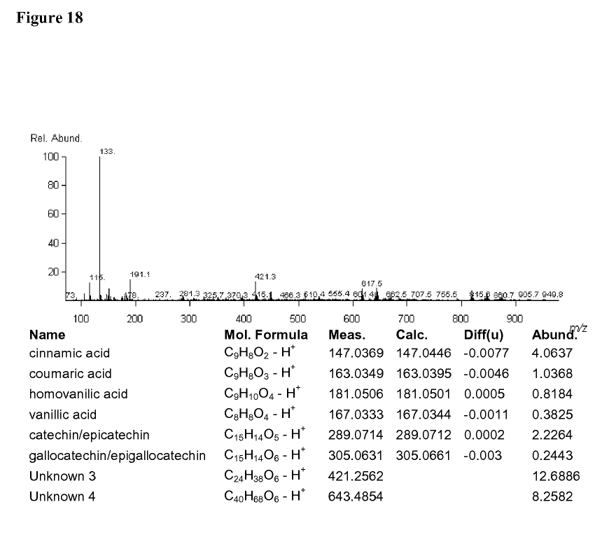

FIG. 18 depicts AccuTOF-DART Mass Spectrum for crude extract of cinnamon bark HS#147 using a 75% EtOH extraction solvent (negative ion mode).

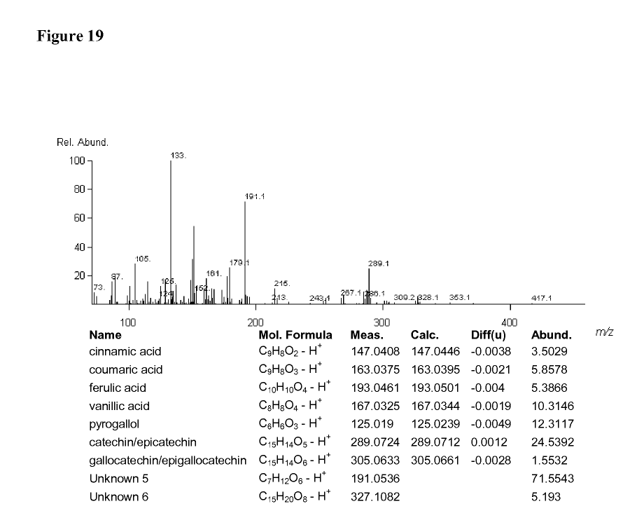

FIG. 19 depicts AccuTOF-DART Mass Spectrum for crude extract of cinnamon bark separated by column chromatography using Sephadex LH-20 packing material (negative ion mode).

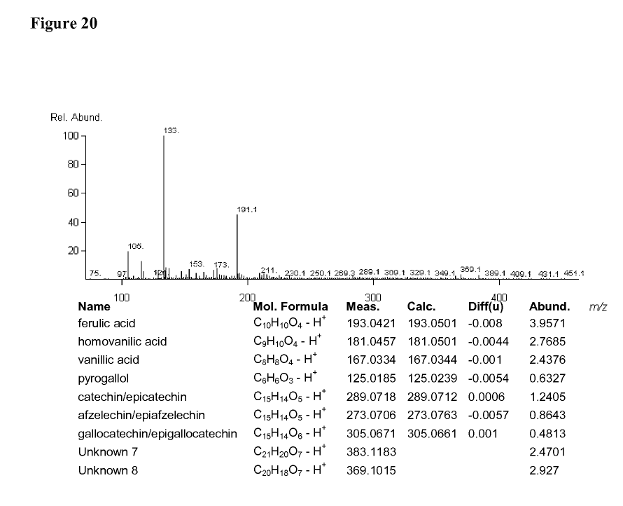

FIG. 20 depicts AccuTOF-DART Mass Spectrum for fraction F3 separated by column chromatography using Sephadex LH-20 packing material (negative ion mode).

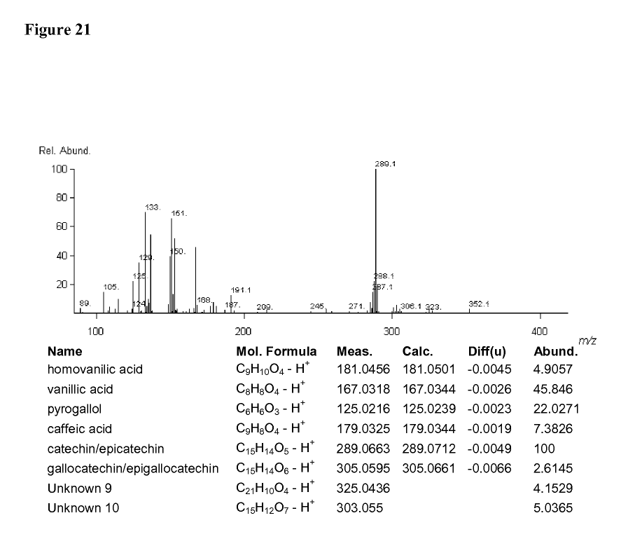

FIG. 21 depicts AccuTOF-DART Mass Spectrum for fraction F4 by column chromatography using Sephadex LH-20 packing material (negative ion mode).

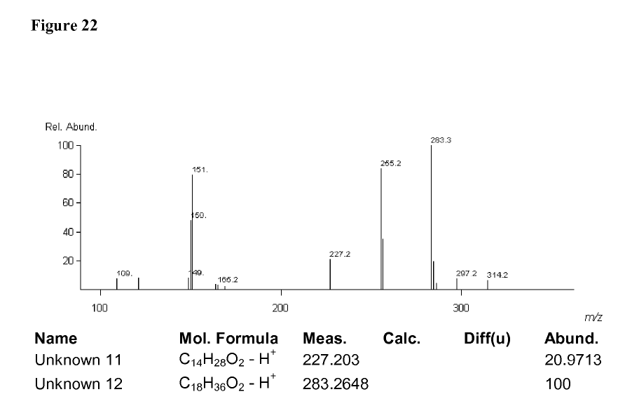

FIG. 22 depicts AccuTOF-DART Mass Spectrum for fraction F5 by column chromatography using Sephadex LH-20 packing material (negative ion mode).

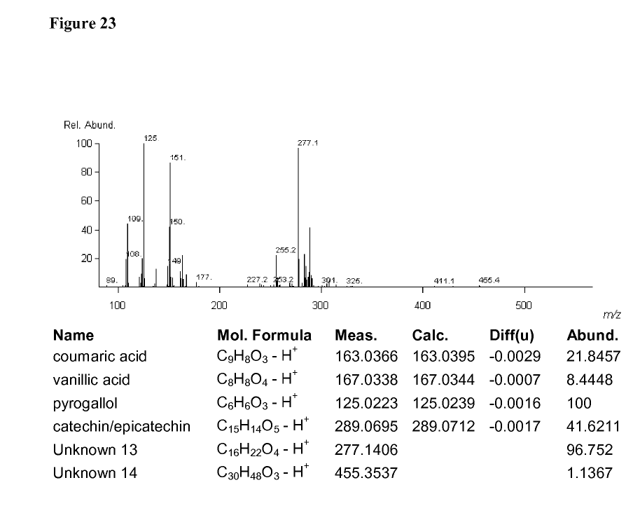

FIG. 23 depicts AccuTOF-DART Mass Spectrum for fraction F6 by column chromatography using Sephadex LH-20 packing material (negative ion mode).

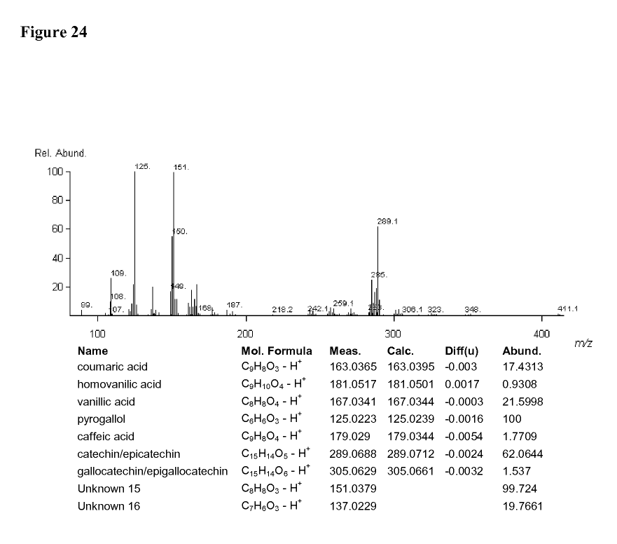

FIG. 24 depicts AccuTOF-DART Mass Spectrum for fraction F7 by column chromatography using Sephadex LH-20 packing material (negative ion mode).

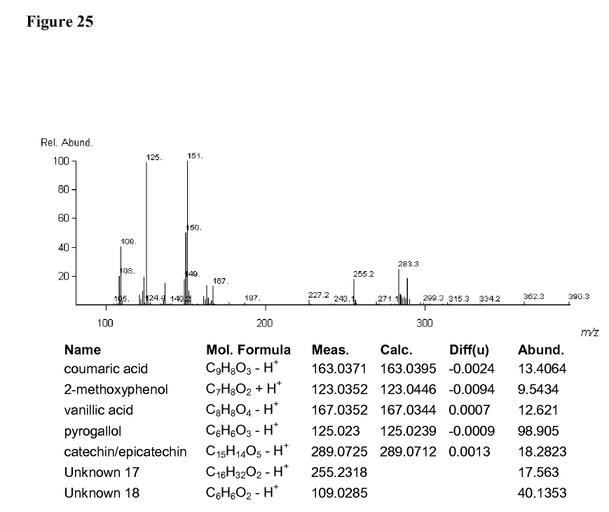

FIG. 25 depicts AccuTOF-DART Mass Spectrum for fraction F8 by column chromatography using Sephadex LH-20 packing material (negative ion mode).

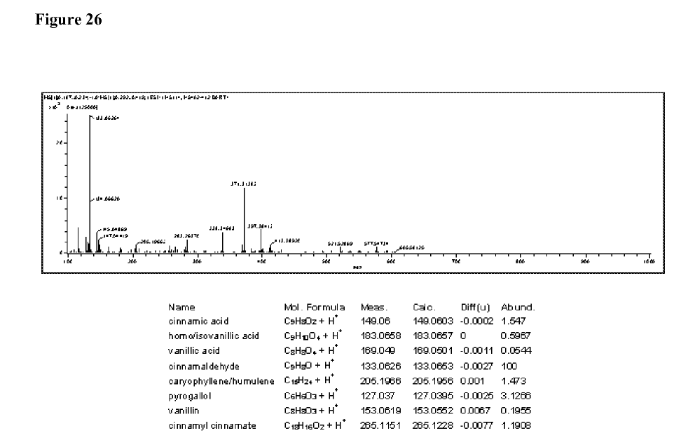

FIG. 26 depicts AccuTOF-DART Mass Spectrum for cinnamon stick purchased commercially from Mountain Rose Herbs (positive ion mode).

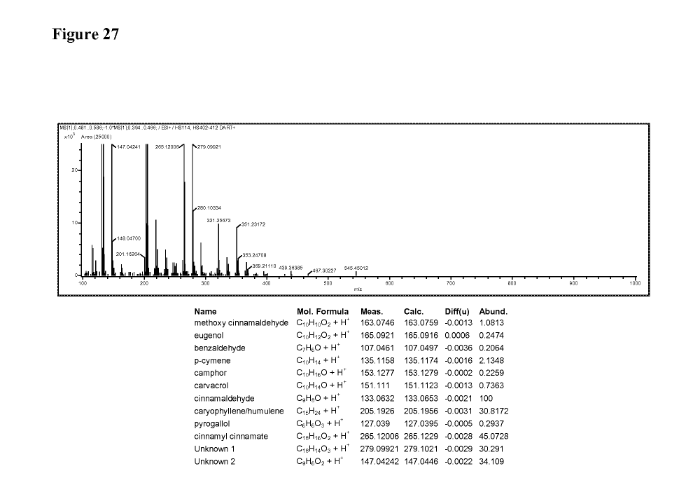

FIG. 27 depicts AccuTOF-DART Mass Spectrum for cinnamon essential oil extracted by SCCO₂ methods at 40° C. and 100 bar (positive ion mode).

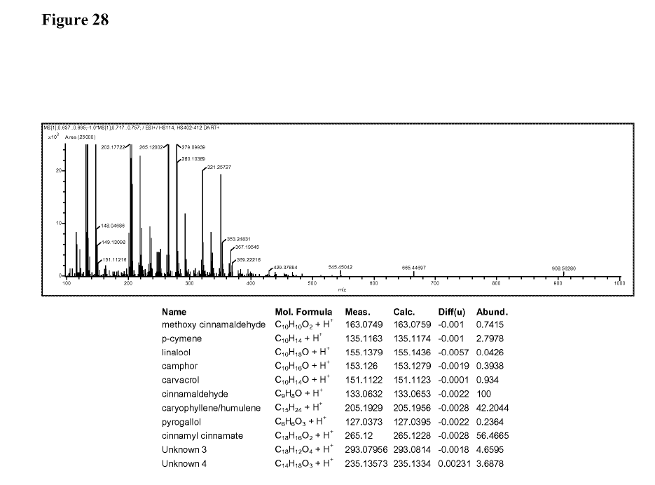

FIG. 28 depicts AccuTOF-DART Mass Spectrum for cinnamon essential oil extracted by SCCO₂ methods at 40° C. and 300 bar (positive ion mode).

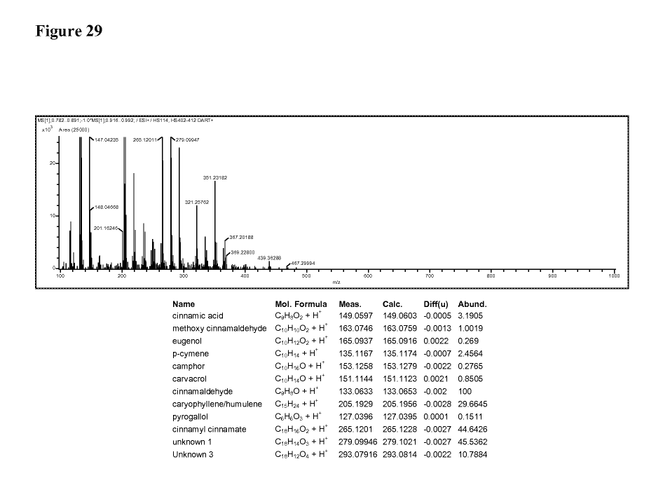

FIG. 29 depicts AccuTOF-DART Mass Spectrum for cinnamon essential oil extracted by SCCO₂ methods at 40° C. and 500 bar (positive ion mode).

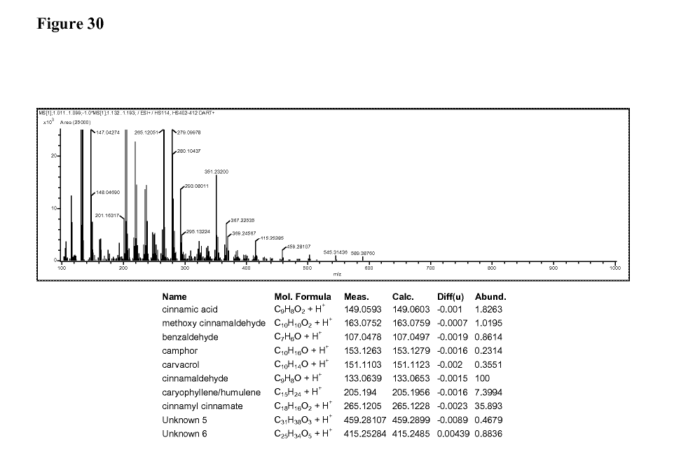

FIG. 30 depicts AccuTOF-DART Mass Spectrum for cinnamon essential oil extracted by SCCO₂ methods at 60° C. and 100 bar (positive ion mode).

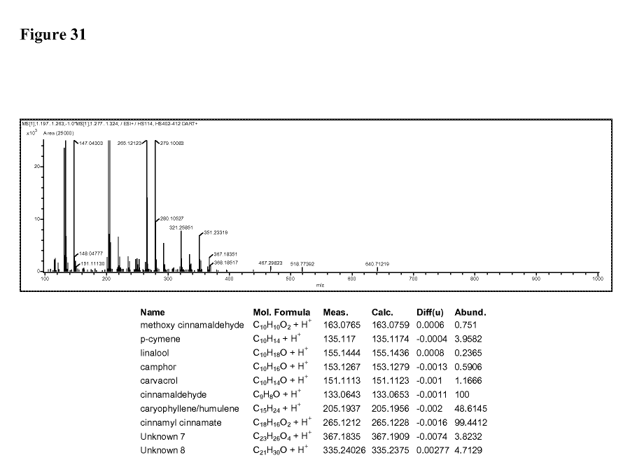

FIG. 31 depicts AccuTOF-DART Mass Spectrum for cinnamon essential oil extracted by SCCO₂ methods at 60° C. and 300 bar (positive ion mode).

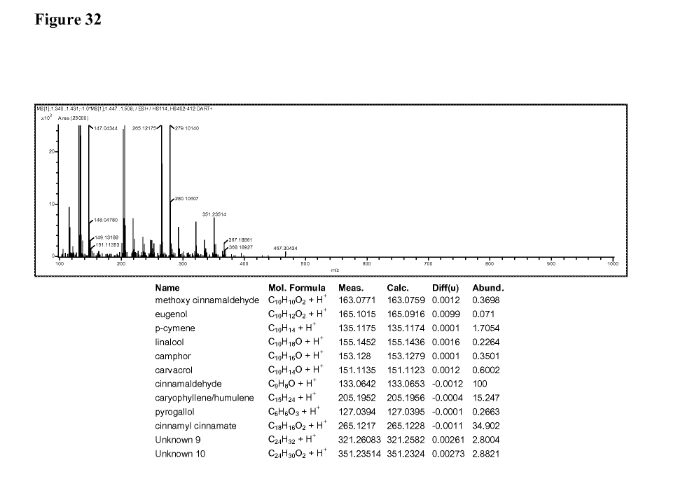

FIG. 32 depicts AccuTOF-DART Mass Spectrum for cinnamon essential oil extracted by SCCO₂ methods at 60° C. and 500 bar (positive ion mode).

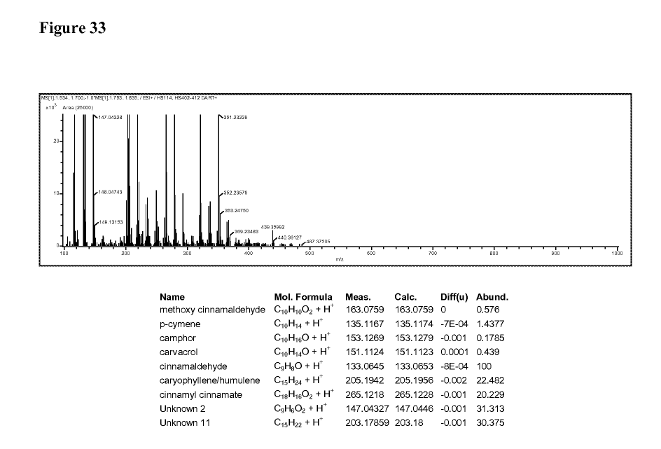

FIG. 33 depicts AccuTOF-DART Mass Spectrum for cinnamon essential oil extracted by SCCO₂ methods at 80° C. and 100 bar (positive ion mode).

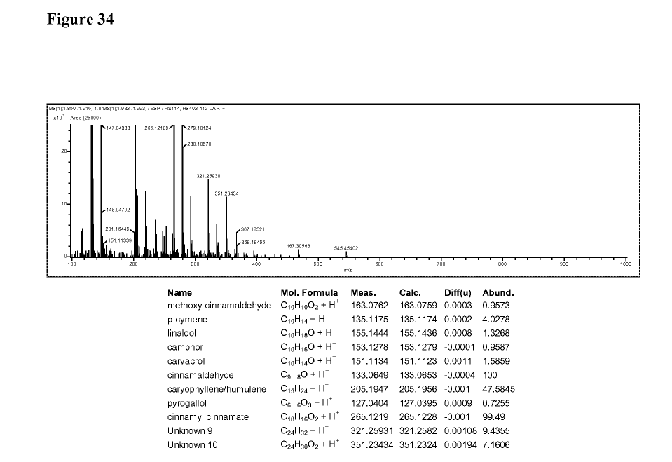

FIG. 34 depicts AccuTOF-DART Mass Spectrum for cinnamon essential oil extracted by SCCO₂ methods at 80° C. and 300 bar (positive ion mode).

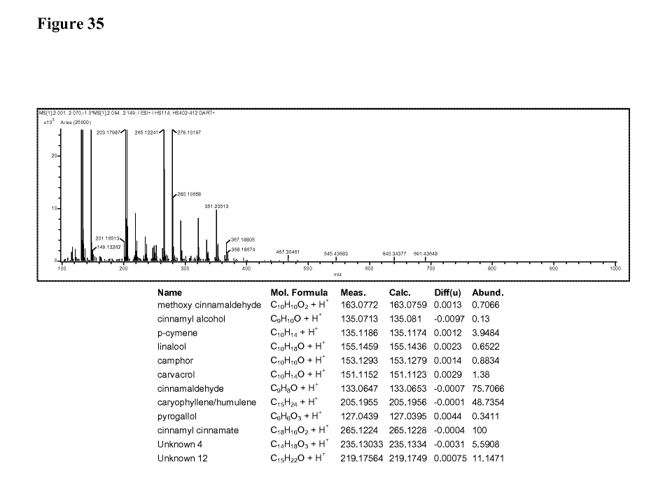

FIG. 35 depicts AccuTOF-DART Mass Spectrum for cinnamon essential oil extracted by SCCO₂ methods at 80° C. and 500 bar (positive ion mode).

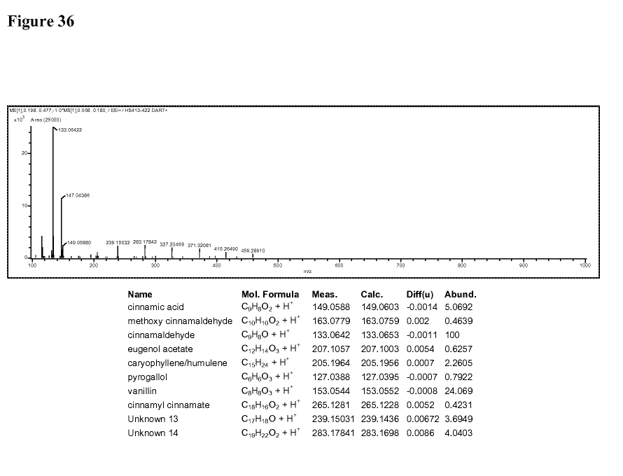

FIG. 36 depicts AccuTOF-DART Mass Spectrum for 80% EtOH leaching extract of crude cinnamon (positive ion mode).

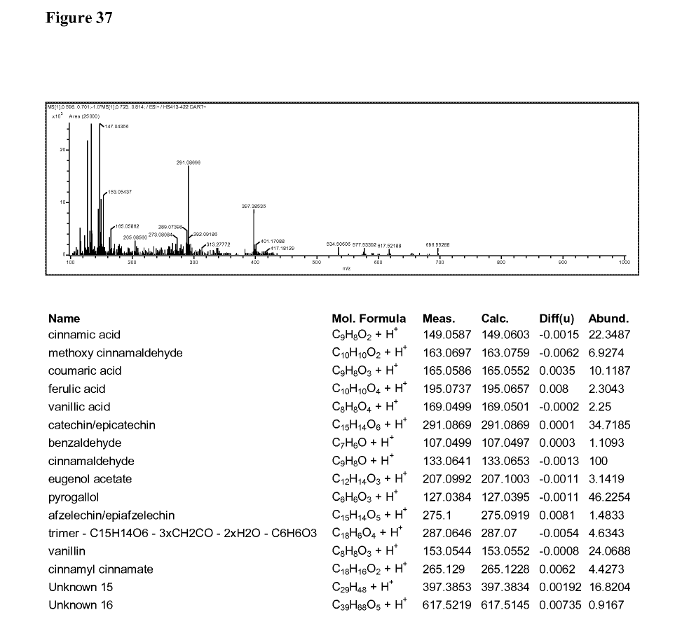

FIG. 37 depicts AccuTOF-DART Mass Spectrum for 80% EtOH leaching extract of residue from SCCO₂ extraction of crude cinnamon (positive ion mode).

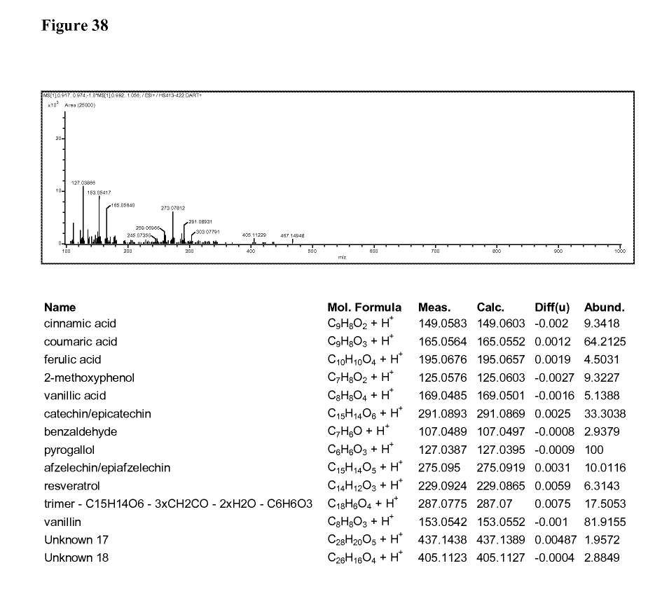

FIG. 38 depicts AccuTOF-DART Mass Spectrum for cinnamon ethanol elution fraction F4 using Sephadex LH-20 packing material of HS114 SCCO₂ residue (positive ion mode).

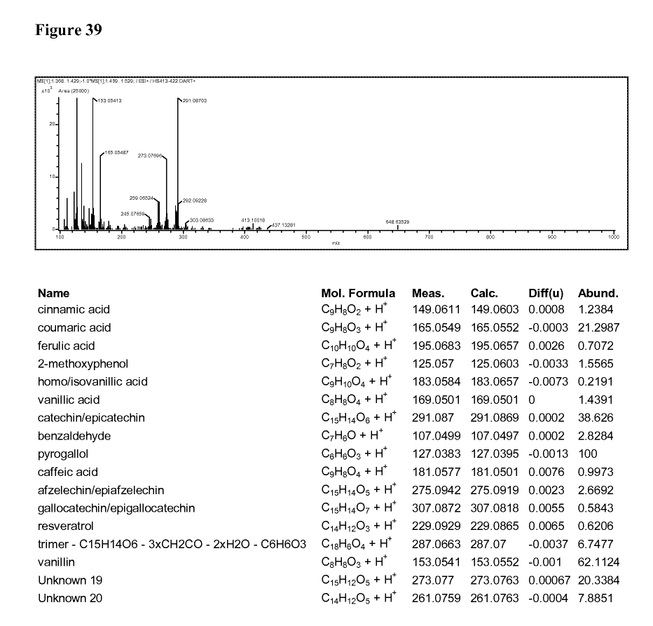

FIG. 39 depicts AccuTOF-DART Mass Spectrum for cinnamon ethanol elution fraction F5 using Sephadex LH-20 packing material of HS114 SCCO₂ residue (positive ion mode).

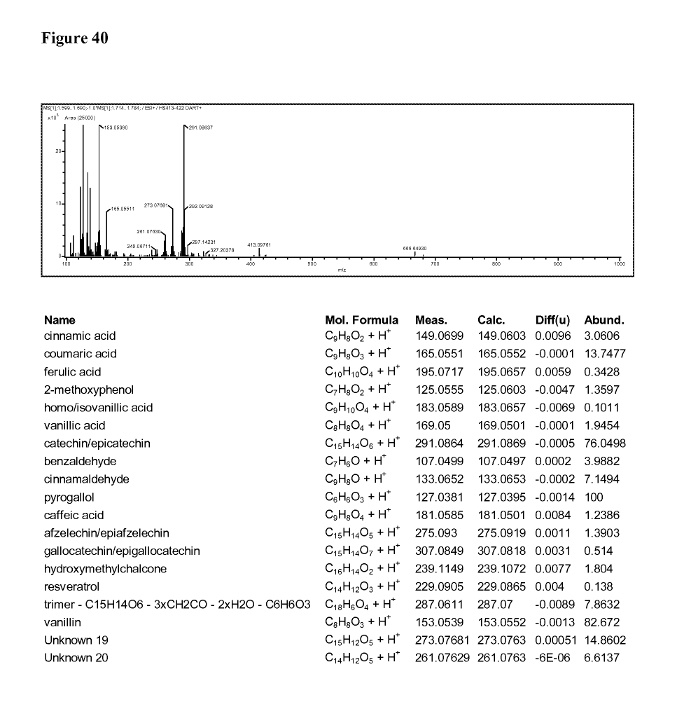

FIG. 40 depicts AccuTOF-DART Mass Spectrum for cinnamon ethanol elution fraction F6 using Sephadex LH-20 packing material of HS114 SCCO₂ residue (positive ion mode).

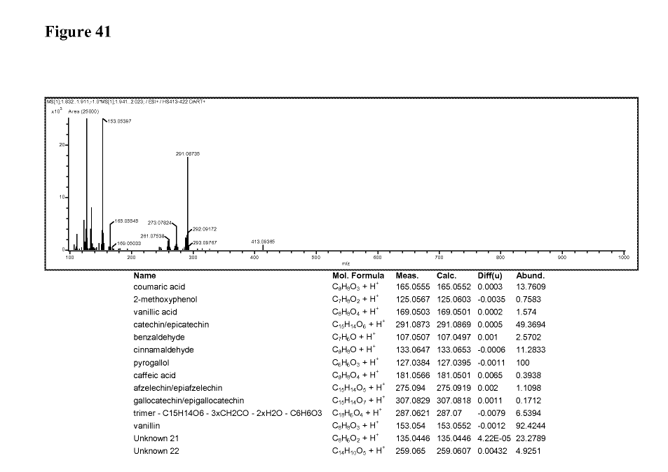

FIG. 41 depicts AccuTOF-DART Mass Spectrum for cinnamon ethanol elution fraction F7 using Sephadex LH-20 packing material of HS114 SCCO₂ residue (positive ion mode).

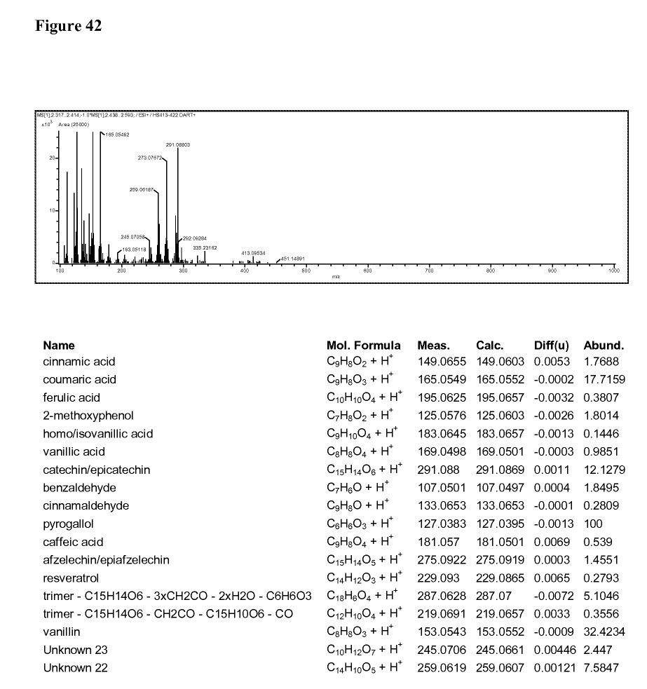

FIG. 42 depicts AccuTOF-DART Mass Spectrum for cinnamon ethanol elution fraction F8 using Sephadex LH-20 packing material of HS114 SCCO₂ residue (positive ion mode).

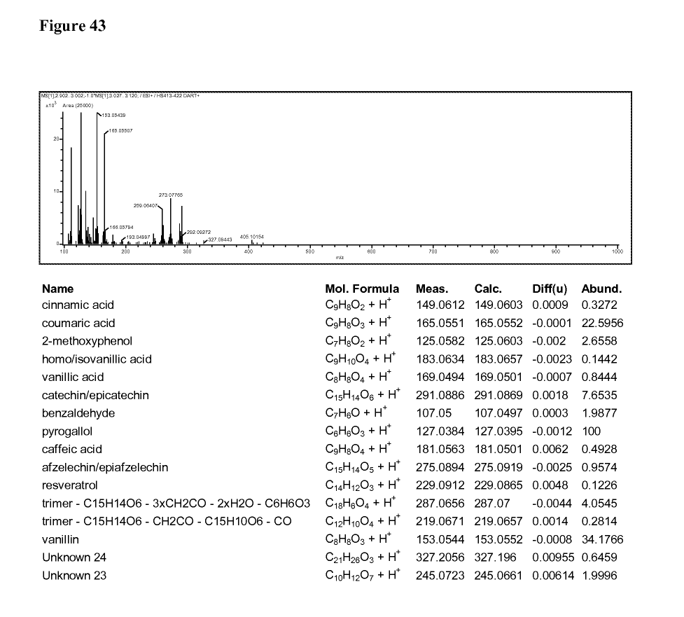

FIG. 43 depicts AccuTOF-DART Mass Spectrum for cinnamon ethanol elution fraction F9 using Sephadex LH-20 packing material of HS114 SCCO₂ residue (positive ion mode).

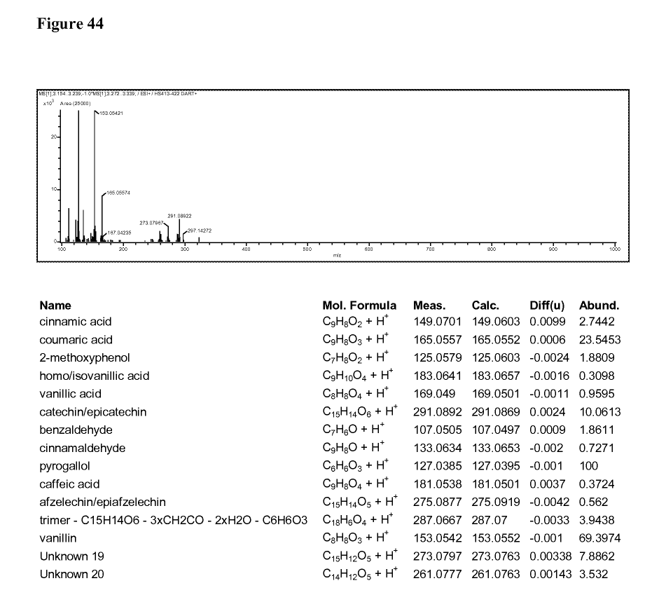

FIG. 44 depicts AccuTOF-DART Mass Spectrum for cinnamon ethanol elution fraction F10 using Sephadex LH-20 packing material of HS114 SCCO₂ residue (positive ion mode).

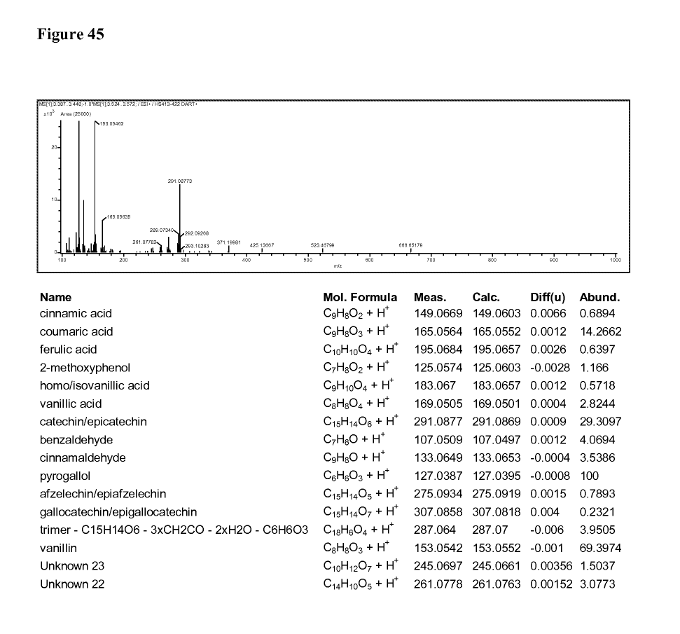

FIG. 45 depicts AccuTOF-DART Mass Spectrum for cinnamon ethanol elution fraction F11 using Sephadex LH-20 packing material of HS114 SCCO₂ residue (positive ion mode).

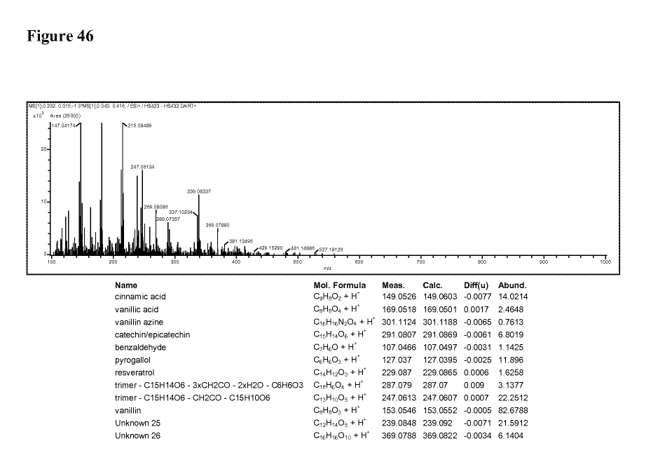

FIG. 46 depicts AccuTOF-DART Mass Spectrum for cinnamon crude extract from HS114 (positive ion mode).

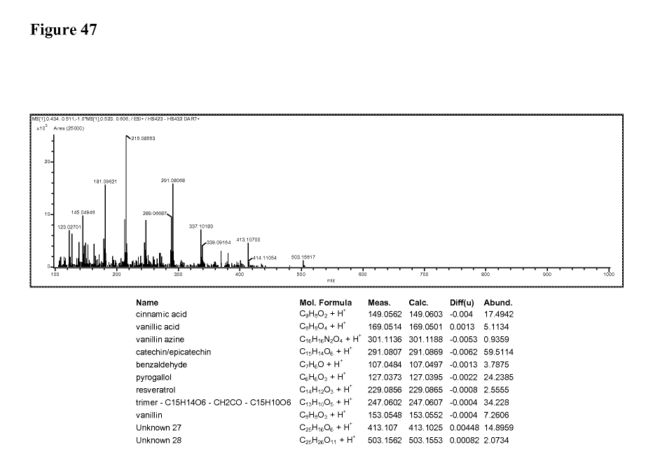

FIG. 47 depicts AccuTOF-DART Mass Spectrum for cinnamon crude extract from HS114 (SCCO₂) (positive ion mode).

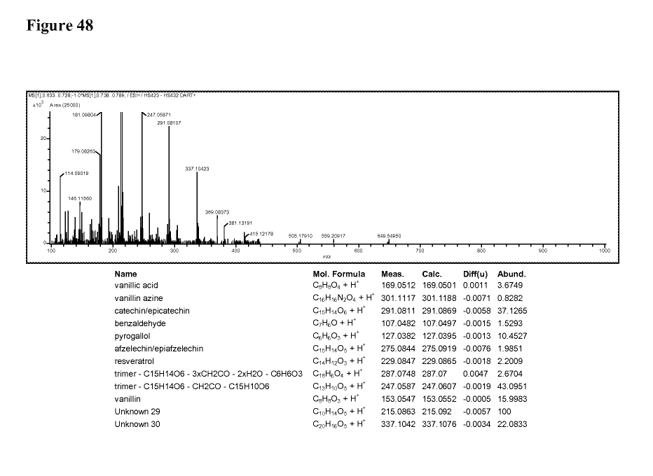

FIG. 48 depicts AccuTOF-DART Mass Spectrum for cinnamon ethanol elution fraction F4 after thiolytic degradation from Sepadex LH-20 (positive ion mode).

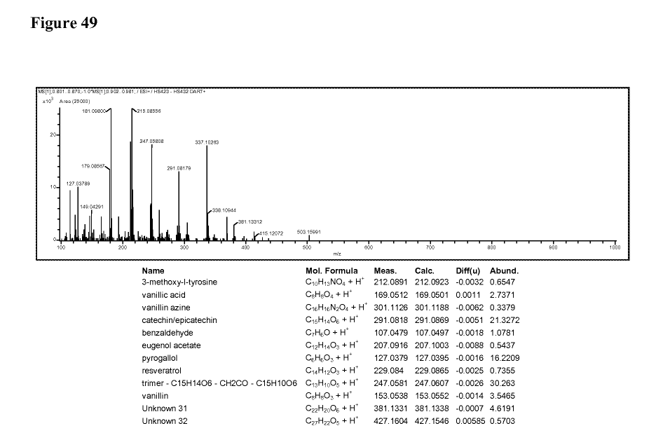

FIG. 49 depicts AccuTOF-DART Mass Spectrum for cinnamon ethanol elution fraction F5 after thiolytic degradation from Sepadex LH-20 (positive ion mode).

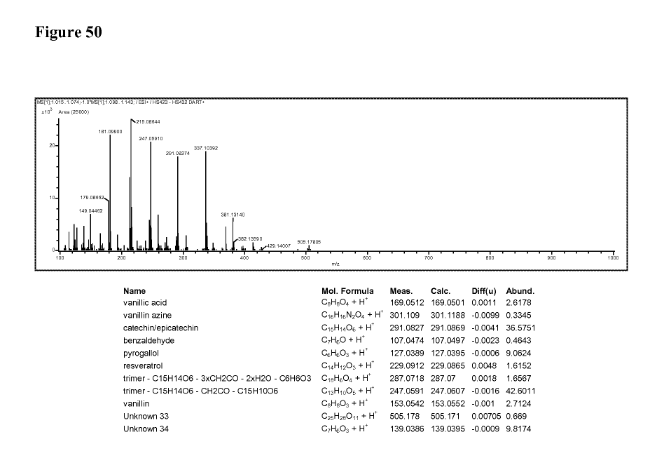

FIG. 50 depicts AccuTOF-DART Mass Spectrum for cinnamon ethanol elution fraction F6 after thiolytic degradation from Sepadex LH-20 (positive ion mode).

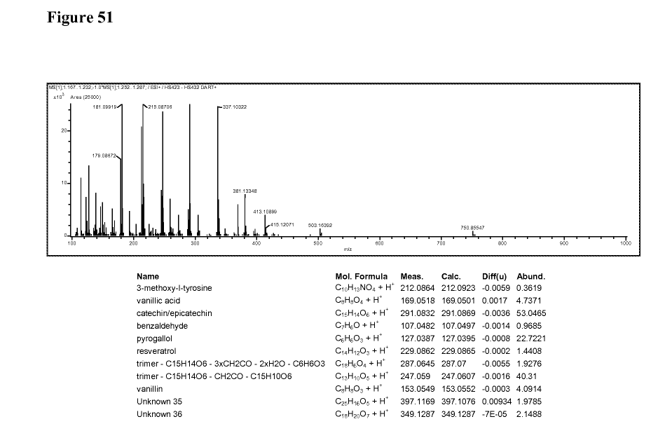

FIG. 51 depicts AccuTOF-DART Mass Spectrum for cinnamon ethanol elution fraction F7 after thiolytic degradation from Sepadex LH-20 (positive ion mode).

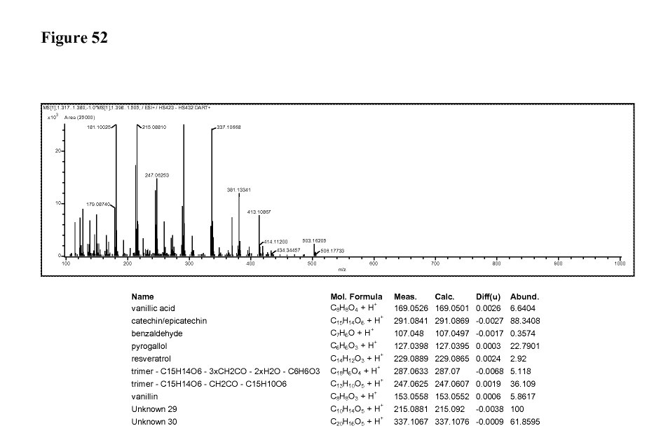

FIG. 52 depicts AccuTOF-DART Mass Spectrum for cinnamon ethanol elution fraction F8 after thiolytic degradation from Sepadex LH-20 (positive ion mode).

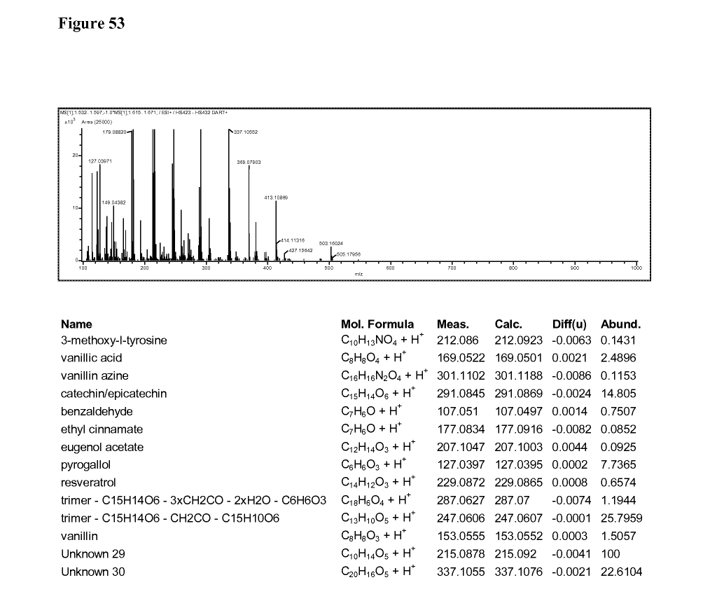

FIG. 53 depicts AccuTOF-DART Mass Spectrum for cinnamon ethanol elution fraction F9 after thiolytic degradation from Sepadex LH-20 (positive ion mode).

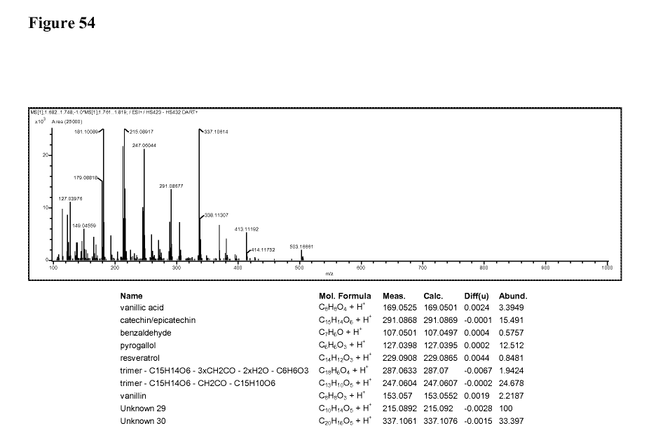

FIG. 54 depicts AccuTOF-DART Mass Spectrum for cinnamon ethanol elution fraction F10 after thiolytic degradation from Sepadex LH-20 (positive ion mode).

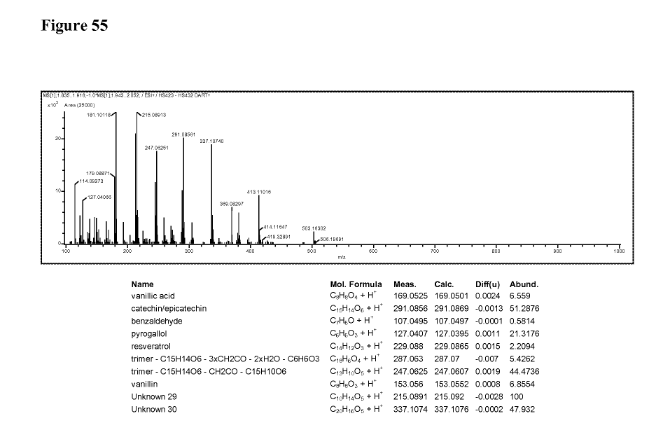

FIG. 55 depicts AccuTOF-DART Mass Spectrum for cinnamon ethanol elution fraction F11 after thiolytic degradation from Sepadex LH-20 (positive ion mode).

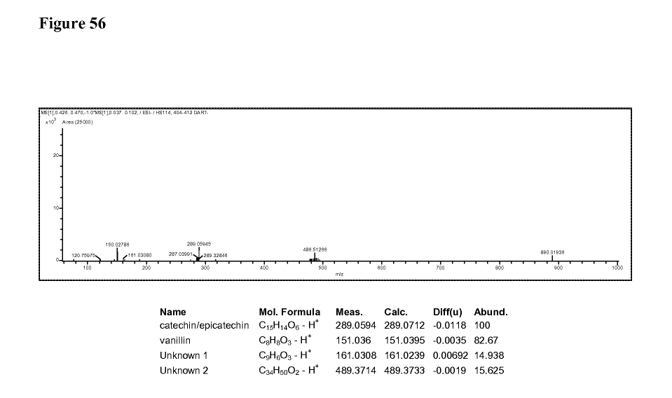

FIG. 56 depicts AccuTOF-DART Mass Spectrum for cinnamon stick purchased commercially from Mountain Rose Herbs (negative ion mode).

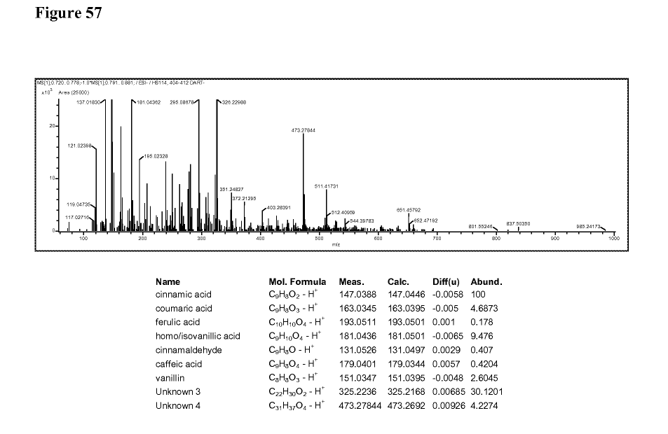

FIG. 57 depicts AccuTOF-DART Mass Spectrum for cinnamon essential oil extracted by SCCO₂ methods at 40° C. and 100 bar (negative ion mode).

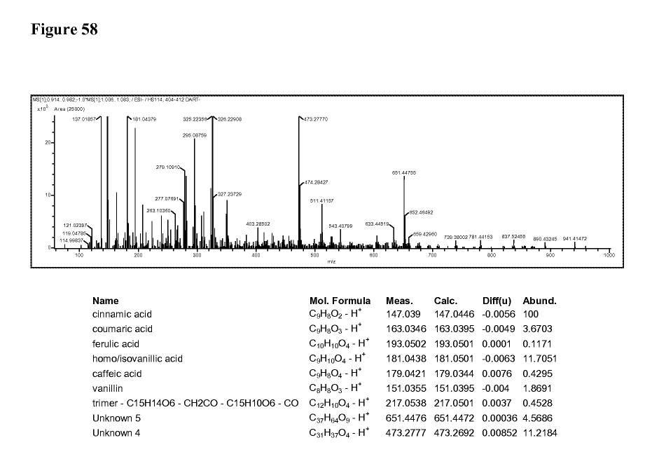

FIG. 58 depicts AccuTOF-DART Mass Spectrum for cinnamon essential oil extracted by SCCO₂ methods at 40° C. and 300 bar (negative ion mode).

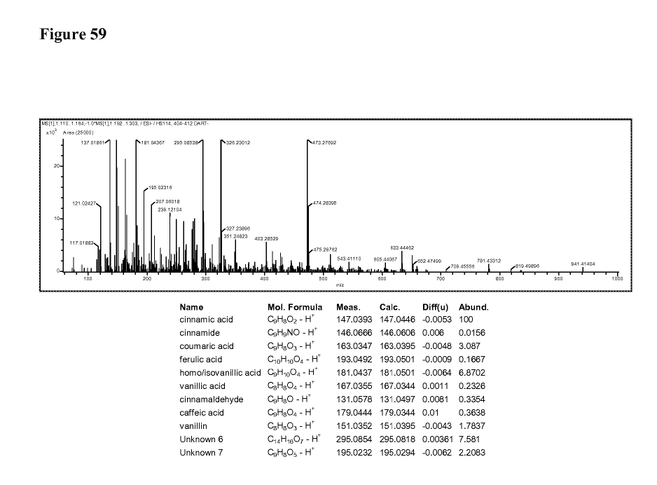

FIG. 59 depicts AccuTOF-DART Mass Spectrum for cinnamon essential oil extracted by SCCO₂ methods at 40° C. and 500 bar (negative ion mode).

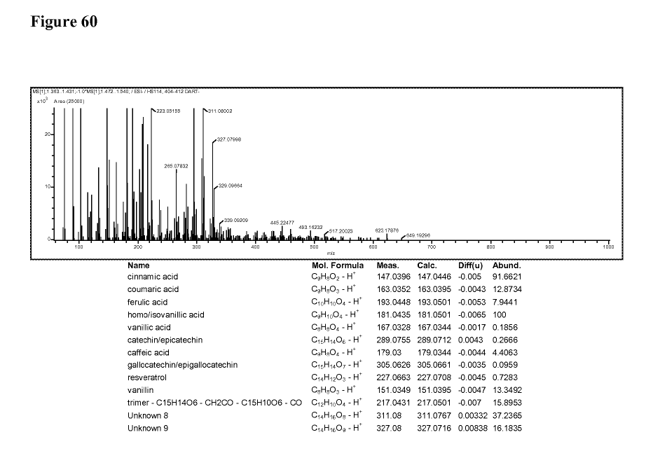

FIG. 60 depicts AccuTOF-DART Mass Spectrum for cinnamon essential oil extracted by SCCO₂ methods at 60° C. and 100 bar (negative ion mode).

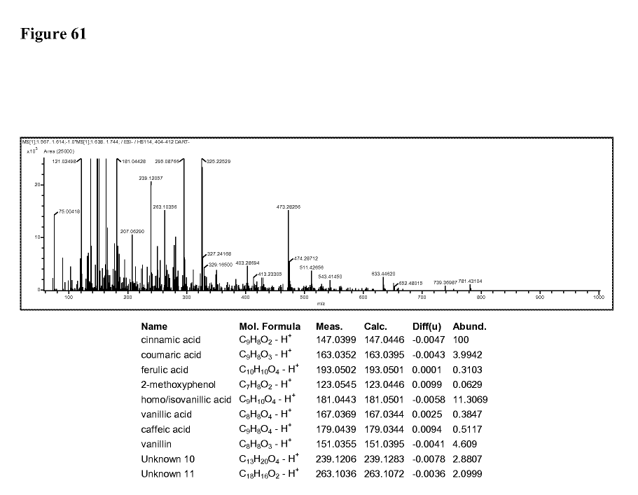

FIG. 61 depicts AccuTOF-DART Mass Spectrum for cinnamon essential oil extracted by SCCO₂ methods at 60° C. and 300 bar (negative ion mode).

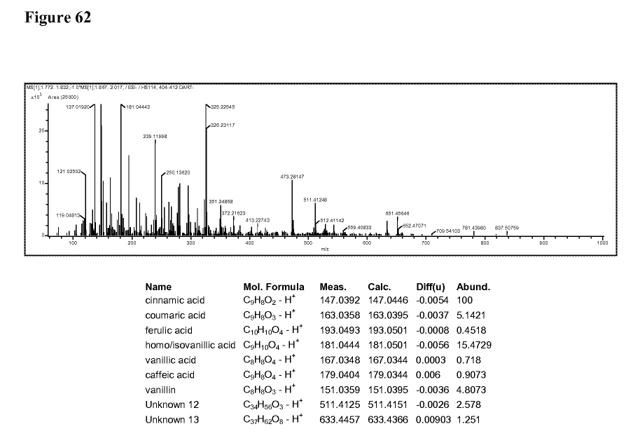

FIG. 62 depicts AccuTOF-DART Mass Spectrum for cinnamon essential oil extracted by SCCO₂ methods at 60° C. and 500 bar (negative ion mode).

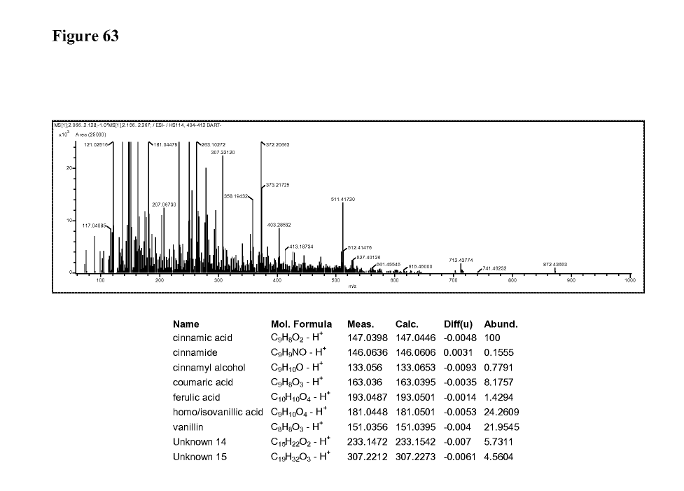

FIG. 63 depicts AccuTOF-DART Mass Spectrum for cinnamon essential oil extracted by SCCO₂ methods at 80° C. and 100 bar (negative ion mode).

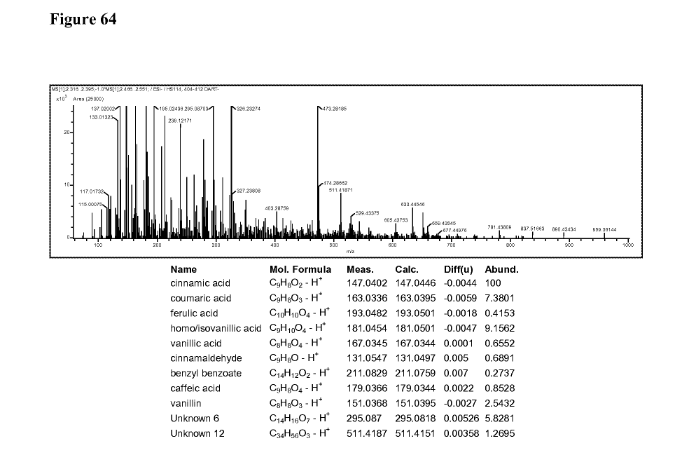

FIG. 64 depicts AccuTOF-DART Mass Spectrum for cinnamon essential oil extracted by SCCO₂ methods at 80° C. and 300 bar (negative ion mode).

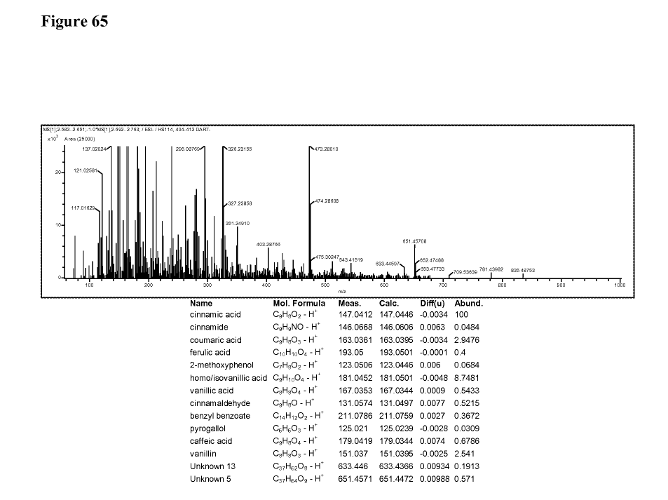

FIG. 65 depicts AccuTOF-DART Mass Spectrum for cinnamon essential oil extracted by SCCO₂ methods at 80° C. and 500 bar (negative ion mode).

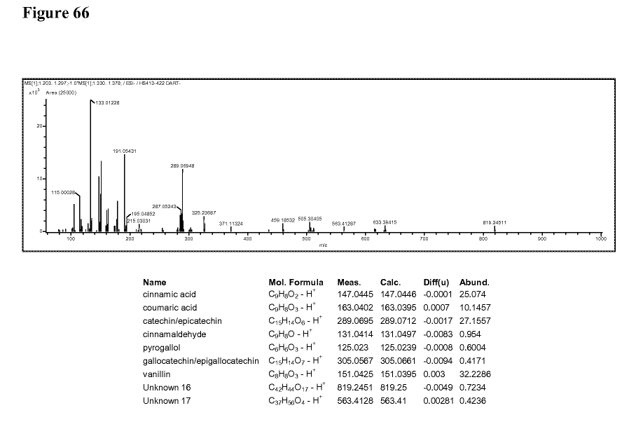

FIG. 66 depicts AccuTOF-DART Mass Spectrum for 80% EtOH leaching extract of crude cinnamon (negative ion mode).

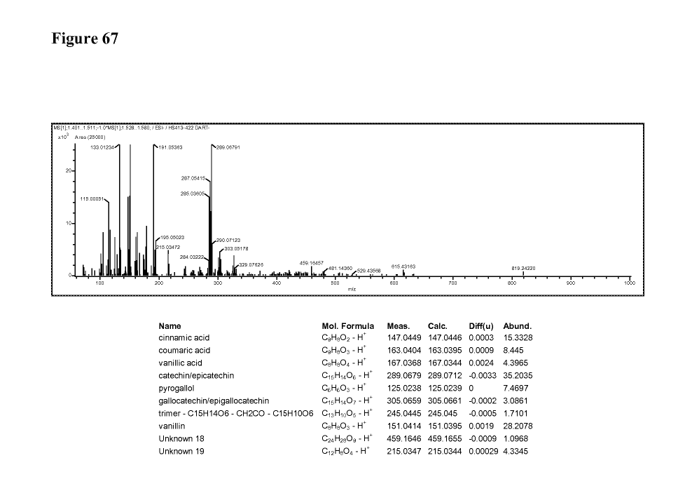

FIG. 67 depicts AccuTOF-DART Mass Spectrum for 80% EtOH leaching extract of residue from SCCO₂ extraction of crude cinnamon (negative ion mode).

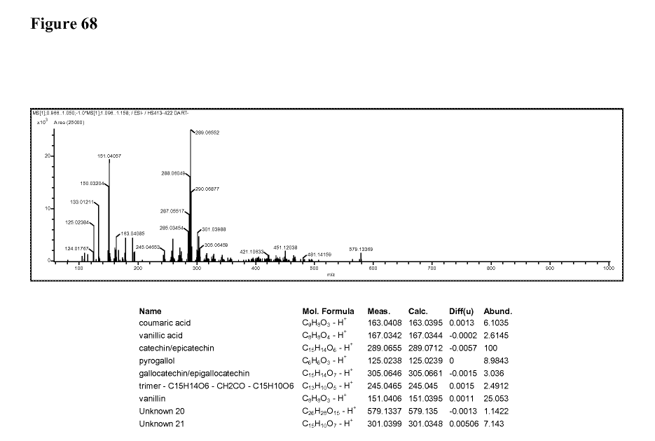

FIG. 68 depicts AccuTOF-DART Mass Spectrum for cinnamon ethanol elution fraction F4 using Sephadex LH-20 packing material of HS114 SCCO₂ residue (negative ion mode).

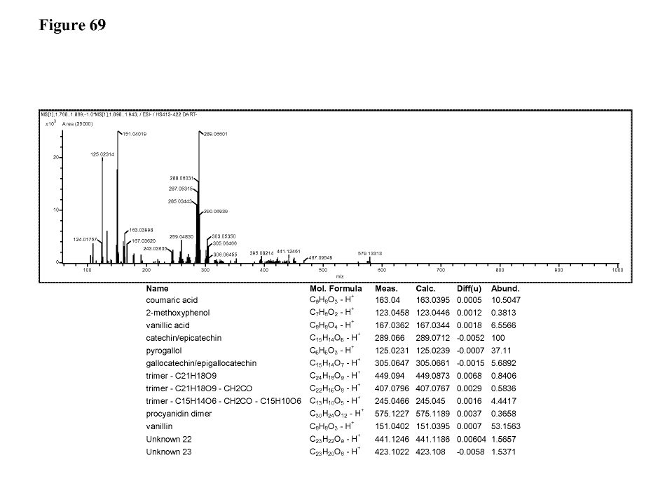

FIG. 69 depicts AccuTOF-DART Mass Spectrum for cinnamon ethanol elution fraction F5 using Sephadex LH-20 packing material of HS114 SCCO₂ residue (negative ion mode).

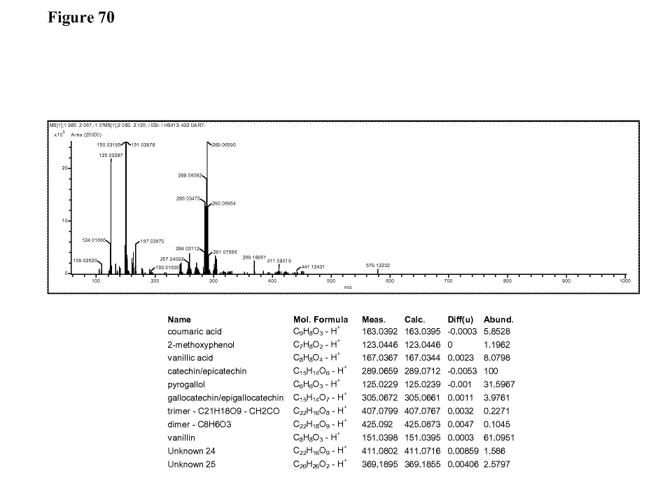

FIG. 70 depicts AccuTOF-DART Mass Spectrum for cinnamon ethanol elution fraction F6 using Sephadex LH-20 packing material of HS114 SCCO₂ residue (negative ion mode).

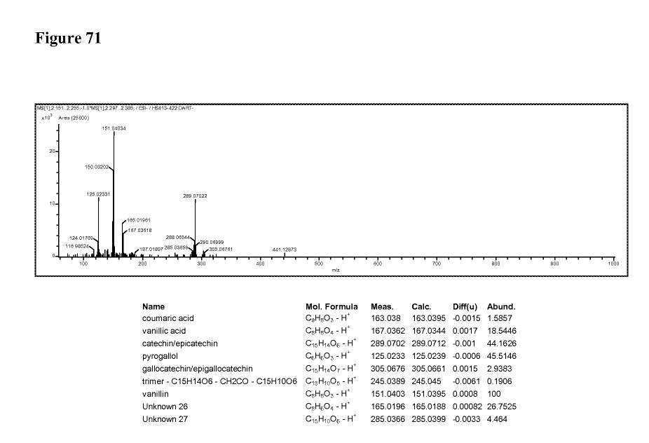

FIG. 71 depicts AccuTOF-DART Mass Spectrum for cinnamon ethanol elution fraction F7 using Sephadex LH-20 packing material of HS114 SCCO₂ residue (negative ion mode).

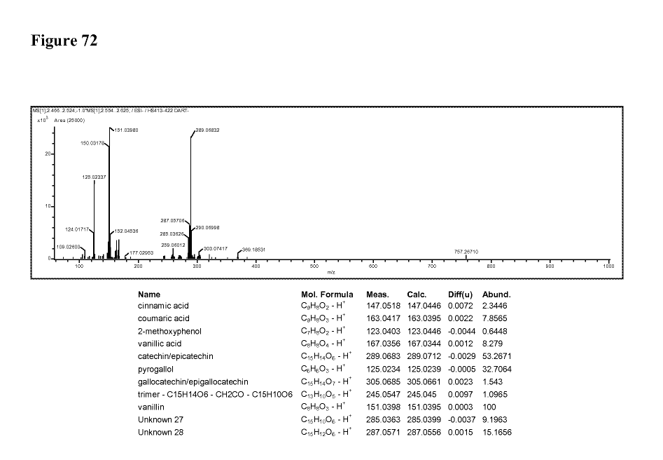

FIG. 72 depicts AccuTOF-DART Mass Spectrum for cinnamon ethanol elution fraction F8 using Sephadex LH-20 packing material of HS114 SCCO₂ residue (negative ion mode).

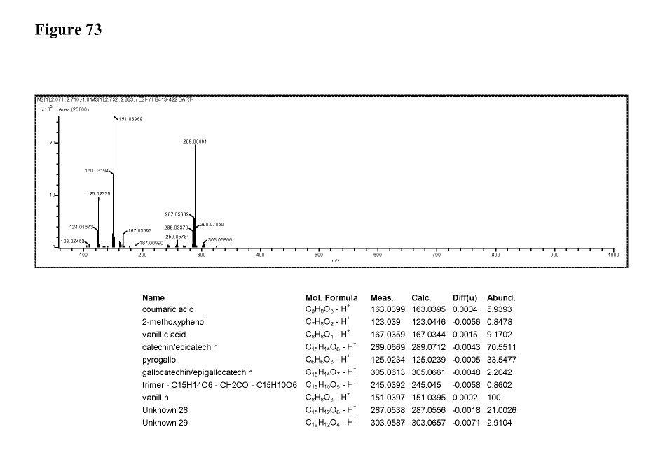

FIG. 73 depicts AccuTOF-DART Mass Spectrum for cinnamon ethanol elution fraction F9 using Sephadex LH-20 packing material of HS114 SCCO₂ residue (negative ion mode).

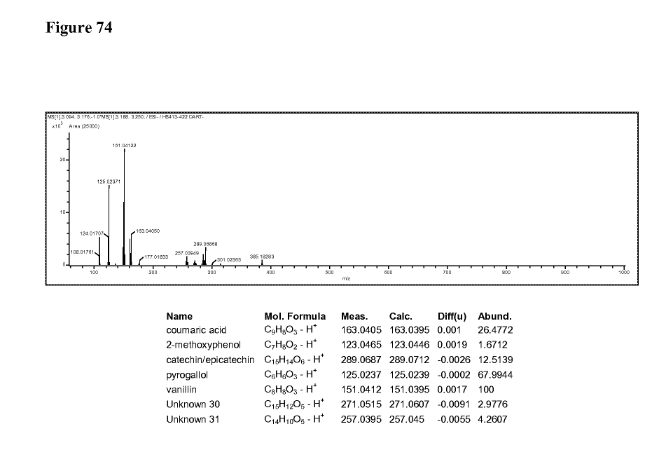

FIG. 74 depicts AccuTOF-DART Mass Spectrum for cinnamon ethanol elution fraction F10 using Sephadex LH-20 packing material of HS114 SCCO₂ residue (negative ion mode).

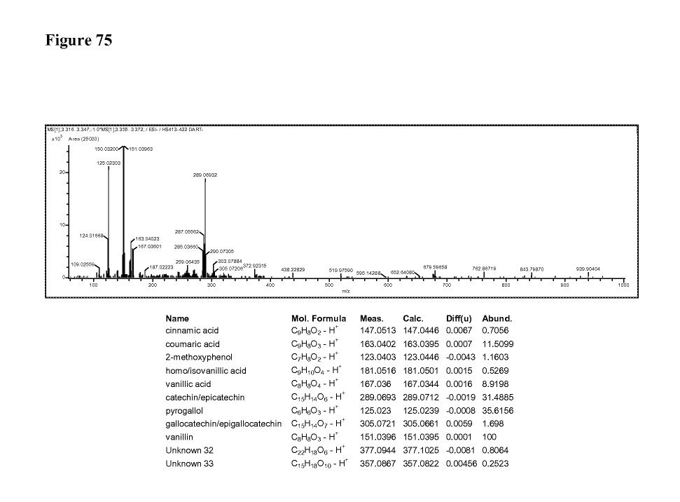

FIG. 75 depicts AccuTOF-DART Mass Spectrum for cinnamon ethanol elution fraction F11 using Sephadex LH-20 packing material of HS114 SCCO₂ residue (negative ion mode).

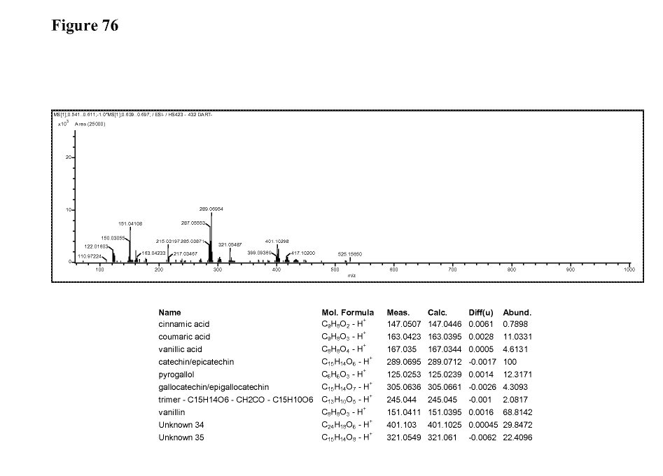

FIG. 76 depicts AccuTOF-DART Mass Spectrum for cinnamon crude extract from HS114 (negative ion mode).

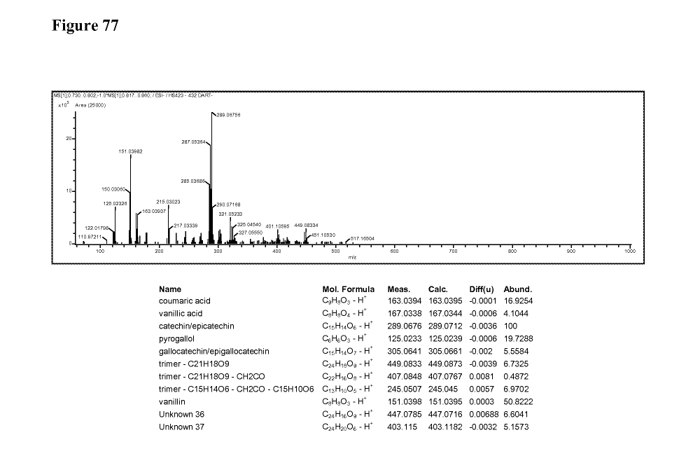

FIG. 77 depicts AccuTOF-DART Mass Spectrum for cinnamon crude extract from HS114 (SCCO₂) (negative ion mode).

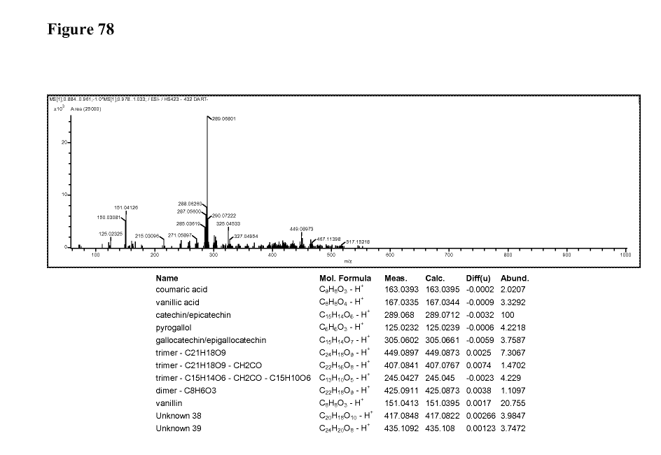

FIG. 78 depicts AccuTOF-DART Mass Spectrum for cinnamon ethanol elution fraction F4 after thiolytic degradation from Sepadex LH-20 (negative ion mode).

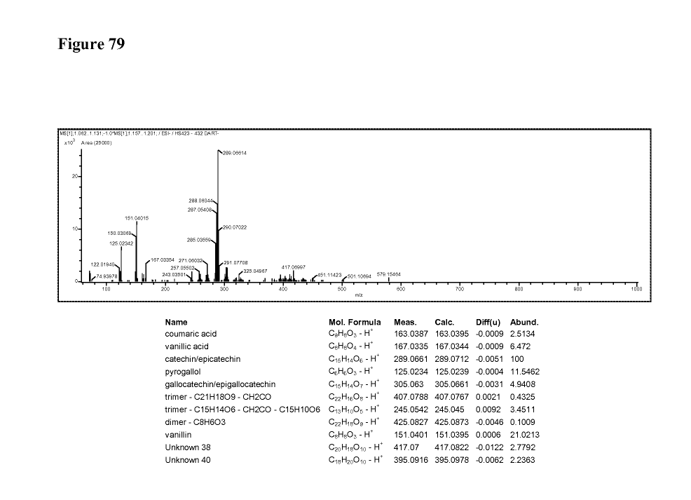

FIG. 79 depicts AccuTOF-DART Mass Spectrum for cinnamon ethanol elution fraction F5 after thiolytic degradation from Sepadex LH-20 (negative ion mode).

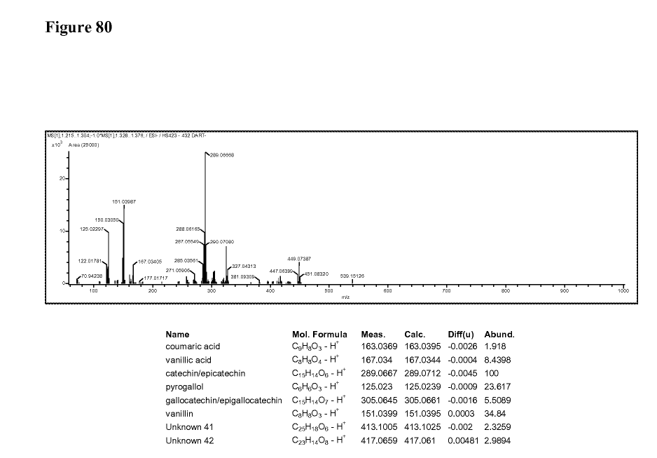

FIG. 80 depicts AccuTOF-DART Mass Spectrum for cinnamon ethanol elution fraction F6 after thiolytic degradation from Sepadex LH-20 (negative ion mode).

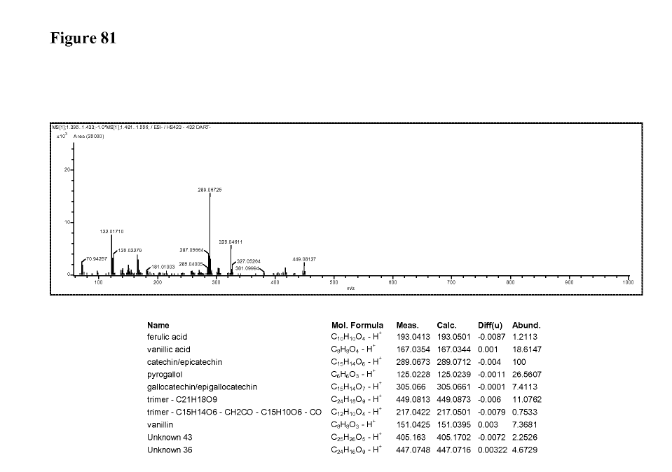

FIG. 81 depicts AccuTOF-DART Mass Spectrum for cinnamon ethanol elution fraction F7 after thiolytic degradation from Sepadex LH-20 (negative ion mode).

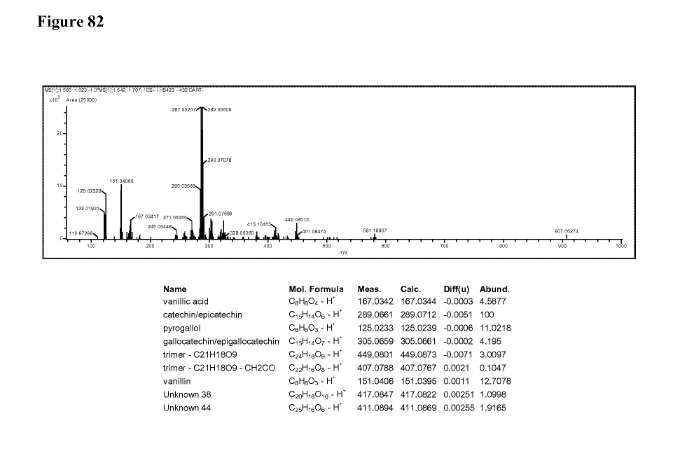

FIG. 82 depicts AccuTOF-DART Mass Spectrum for cinnamon ethanol elution fraction F8 after thiolytic degradation from Sepadex LH-20 (negative ion mode).

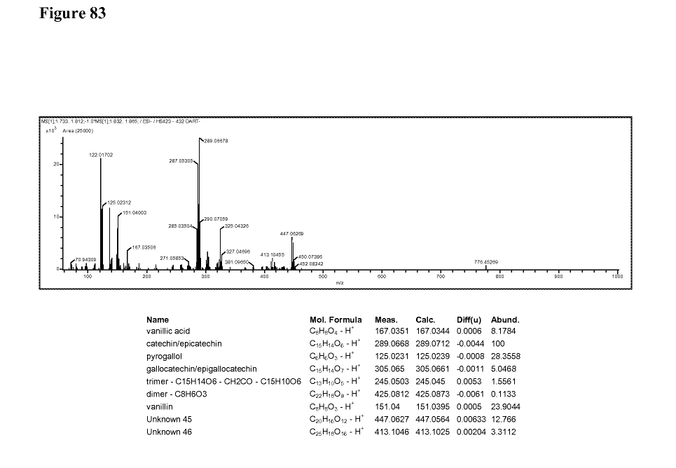

FIG. 83 depicts AccuTOF-DART Mass Spectrum for cinnamon ethanol elution fraction F9 after thiolytic degradation from Sepadex LH-20 (negative ion mode).

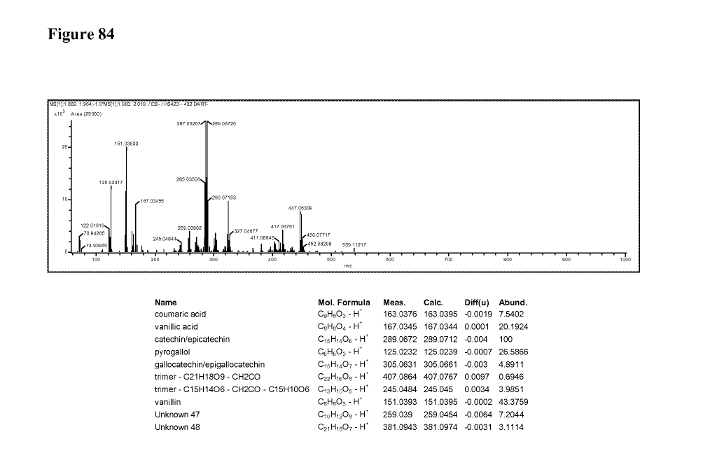

FIG. 84 depicts AccuTOF-DART Mass Spectrum for cinnamon ethanol elution fraction F10 after thiolytic degradation from Sepadex LH-20 (negative ion mode).

FIG. 85 depicts AccuTOF-DART Mass Spectrum for cinnamon ethanol elution fraction F11 after thiolytic degradation from Sepadex LH-20 (negative ion mode).

### DETAILED DESCRIPTION OF THE INVENTION

Definitions

As used herein, cinnamon refers to the bark plant material derived from the Cinnamomum species botanical. The term “cinnamon” is also used interchangeably with cinnamon species and relates to said plants, clones, variants, and sports, etc.

As used herein, the term “one or more compounds” means that at least one compound, such as, but not limited to, trans-cinnamaldehyde (a lipid soluble essential oil chemical constituent of cinnamon species), or methylhydroxychalcone (a water soluble polyphenolic of cinnamon species) or a polysaccharide molecule of cinnamon species is intended, or that more than one compound, for example, trans-cinnamaldehyde and methylhydroxychalcone is intended.

As used herein, the term “fraction” means the extraction comprising a specific group of chemical compounds characterized by certain physical and/or chemical properties.

As used herein, the term “essential oil fraction” refers to a fraction comprising lipid soluble, water insoluble compounds obtained or derived from cinnamon and related species including, but not limited to, the chemical compound classified as trans-cinnamaldehyde.

As used herein, the term “essential oil sub-fraction” refers to a fraction comprising lipid soluble, water insoluble compounds obtained or derived from cinnamon and related species including, but not limited to, the chemical compound classified as trans-cinnamaldehyde having enhanced concentrations of specific compounds found in the essential oil of cinnamon species.

As used herein, the term “polyphenolic fraction” refers to a fraction comprising the water soluble and ethanol soluble polyphenolic acid compounds obtained or derived from cinnamon and related species, further comprising, but not limited to, compounds such as methylhydroxychalcone, and catechin and epicatechin oligomers.

As used herein, the term “polysaccharide fraction” refers to a fraction comprising soluble-ethanol insoluble polysaccharide compounds obtained or derived from cinnamon and related species.

Other chemical constituents of cinnamon may also be present in these extraction fractions.

As used herein, the term “purified” fraction relates to a fraction comprising a specific group of compounds characterized by certain physical-chemical properties or physical or chemical properties that are concentrated to greater than 20% of the fraction's chemical constituents. In other words, a purified fraction comprises less than 80% chemical constituent compounds that are not characterized by certain desired physical-chemical properties or physical or chemical properties that define the fraction.

As used herein, the term “profile” refers to the ratios by percent mass weight of the chemical compounds within an extraction fraction or sub-fraction or to the ratios of the percent mass weight of each of the three cinnamon fraction chemical constituents in a final cinnamon extraction.

As used herein, “feedstock” generally refers to raw plant material, comprising whole plants alone, or in combination with on or more constituent parts of a plant comprising leaves, roots, including, but not limited to, main roots, tail roots, and fiber roots, stems, bark, leaves, seeds, and flowers, wherein the plant or constituent parts may comprise material that is raw, dried, steamed, heated or otherwise subjected to physical processing to facilitate processing, which may further comprise material that is intact, chopped, diced, milled, ground or otherwise processed to affected the size and physical integrity of the plant material. Occasionally, the term “feedstock” may be used to characterize an extraction product that is to be used as feed source for additional extraction processes.

As used herein, the term “cinnamon constituents” shall mean chemical compounds found in cinnamon species and shall include all such chemical compounds identified above as well as other compounds found in cinnamon species, including but not limited to the essential oil chemical constituents, polyphenolic acids, and polysaccharides.

The chemical constituents of cinnamon are of extensive therapeutic value. Recent scientific research and clinical studies have demonstrated the following therapeutic effects of the various chemical compounds, chemical fractions, and gross extraction products of cinnamon which include the following: NIDDM-type 2 diabetes mellitus (proanthocyanidins, methylhydroxychalcone, catechins and epicatechin oligomers, flavonoids, water soluble extract); Improved cholesterol metabolism including decreased low density lipoprotein (phenolic acids including proanthocyanidins, methylhydroxychacone, catechins, epicatechin oigomers, flavonoids, water soluble extract); anti-artery damaging free radicals and improved function of small blood vessels (essential oils, cinnamaldehyde, 2′-hydroxycinnamaldehyde, 2′-methoxycinnmaldehyde, phenolic acids, flavonoids glycosides, proanthocyanidins, flavonoids, catechins, epicatechin oligomers, extract); anti-thrombotic and anti-platelet aggregation (essential oil, cinnamaldehyde); anti-inflammatory activity (essential oil, cinnamaldehyde, eugenol, 1,8 cineole, alpha-pinene, beta-pinene, borneol, flavonol glycosides, extract); anti-oxidant (phenolic acids, flavonol glycosides, proanthocyanidins, flavonoids, water soluble extract); anti-allergic (phenolic acids, flavonol glycosides, proanthocyanidins, flavonoids, water soluble extract); Neurological protectant (water soluble extract); cardiovascular protectant (essential oil, water soluble extract); enhanced brain function (essential oil, particularly volatile oils); caminative, loss of appetite, dyspeptive complaints, anti-vomiting, anti-bloating & flatulence, promotion of intestinal motility, facilitation of weight gain, (flavonoids, 3-(2-hydroxyphenyl)-propanoic acid, 3-(2-hydroxyphenyl)-O-glycoside, water soluble extract); anti-cough, common cold and fever (essential oil, cinnamyl acetate); anti-bacterial & anti-fungal activity (essential oil, cinnamaldehyde, eugenol, 1,8-cineole, beta-pinene, borneol); lipolytic & improved wound healing (ethanol extract); and anti-cancer & anti-gout (essential oil, cinnamaldehyde, 2′-hydroxycinnamaldehyde, 2′-benzoxycinnamaldehyde, methanol extract); See Khan A et al. Diabetes Care 26:3215-3218, 2003; Anderson R A et al. J Agric Food Chem 52:65-70, 2004; Jarville-Taylor et al. J Am Coll Nutri 20:327-336, 2001; Qin R et al. Horm Metab Res 36:119-123, 2004; Vespohl E J et al. Phytother Res 19:203-206, 2005; Lee S H et al Biochem Pharmacol 69:791-9, 2005; Chericoni S et al. J Agric Food Chem 53:4762-4765, 2005; Lin C C et al. Phytother Res 17:7260730, 2003; Jayaprakasha G K et al. J Agric Food Chem 51:4344-4348, 2003; Huss U et al. J Nat Prod 65:1517-21, 2002; Nagai H et al. Jpn J Pharmacol 32:813-822, 1982; Su M J et al. J Biomed Sci 6:376-386, 1999; Shimada Y et al. Phytomed 11:404-410, 2004; Taher M et al. Med J Malayia 59B:97-98, 2004; Kamath J V et al. Phytother Res 17:970-972, 2003; Kurokawa M et al. Eur J Pharmacol 348:45-51, 1998; Simic A et al. Phytother Res 18:713-717, 2004; Tabak M et al. J Ethnopharmacol 67:269-277, 1999; Kong L D et al. J Ethnopharmacol 73:199-207, 2000; Kwon B M et al. Arch Pharm Res 21:147-152, 1998; Ka H et al. Cancer Lett 196:143-152, 2003.

Anthocyanins are a particular class of naturally occurring flavonoid compounds that are responsible for the red, purple, and blue colors of many fruits, vegetables, cereal grains, and flowers. For example, the colors of fruits such as blueberries, bilberries, strawberries, raspberries, boysenberries, marionberries, cranberries, elderberries, etc. are due to many different anthocyanins. Recently, the interest in anthocyanin pigments has intensified because of their possible health benefits as dietary antioxidants. For example, anthocyanin pigments of bilberries (Vaccinium myrtillus) have long been used for improving visual acuity and treating circulatory disorders. There is experimental evidence that certain anthocyanins and other flavonoids have anti-inflammatory properties. In addition, there are reports that orally administered anthocyanins are beneficial for treating diabetes and ulcers and may have antiviral and antimicrobial activities. The chemical basis for these desirable properties of flavonoids is believed to be related to their antioxidant capacity. Thus, the antioxidant characteristics associated with berries and other fruits and vegetables have been attributed to their anthocyanin content.

Proanthocyanidins, also known as “oligomeric proanthocyanidins,” “OPCs,” or “procyanidins,” are another class of naturally occurring flavonoid compounds widely available in fruits, vegetables, nuts, seeds, flowers, and barks. Proanthocyanidins belong to the category known as condensed tannins. They are the most common type of tannins found in fruits and vegetables, and are present in large quantities in the seeds and skins. In nature, mixtures of different proanthocyanidins are commonly found together, ranging from individual units to complex molecules (oligomers or polymers) of many linked units. The general chemical structure of a polymeric proanthocyanidin comprises linear chains of flavonoid 3-ol units linked together through common C(4)-C(6) and/or C(4)-C(8) bonds. The proanthocyanidins are mixtures of oligomers and polymers containing catechin and/or epicatechin units linked through C4-C8 and/or C4-C6 bonds. These flavan-3-ols can also be doubly linked by a C4-C8 bond and an additional ether bond between C7-C2. ¹³C NMR has been useful in identifying the structures of polymeric proanthocyanidins, and recent work has elucidated the chemistry of di-, tri-, and tetrameric proanthocyanidins. Larger oligomers of the flavonoid 3-ol units are predominant in most plants and are found with average molecular weights above 2,000 Daltons and containing 6 or more monomer units. (Newman, et al., Mag. Res. Chem., 25:118 (1987)). Considerable recent research has explored the therapeutic applications of proanthocyanidins, which are primarily known for their antioxidant activity. However, these compounds have also been reported to demonstrate antibacterial, antiviral, anticarcinogenic, anti-inflammatory, anti-allergic, and vasodilatory actions. In addition, they have been found to inhibit lipid peroxidation, platelet aggregation, capillary permeability and fragility, and to affect enzyme systems including phospholipase A2, cyclooxygenase, and lipoxygenase. For example, proanthocyanidin monomers (i.e., anthocyanins) and dimers have been used in the treatment of diseases associated with increased capillary fragility and have also been shown to have anti-inflammatory effects in animals (Beladi, I. et al., Ann. N.Y. Acad. Sci., 284:358 (1977)). Based on these reported findings, oligomeric proanthocyanidins (OPCs) may be useful components in the treatment of a number of conditions (Fine, A. M., Altern. Med. Rev. 5(2):144-151 (2000)).

Proanthocyanidins may also protect against viruses. In in vitro studies, proanthocyanidins from witch hazel (Hamamelis virginiana) killed the Herpes simplex 1 (HSV-1) virus (Erdelmeier, C. A., Cinatl, J., Plant Med. June: 62(3):241-5 (1996); DeBruyne, T., Pieters, L., J. Nat. Prod. July: 62(7):954-8 (1999)). Another study was carried out to determine the structure-activity relationships of the antiviral activity of various tannins. It was found that the more condensed the chemical structure, the greater the antiviral effect (Takechi, M., et al., Phytochemistry, 24:2245-50 (1985)). In another study, proanthocyanidins were shown to have anti-Herpes simplex activity in which the 50 percent effective doses needed to reduce herpes simplex plaque formation were two to three orders of magnitude less than the 50 percent cytotoxic doses (Fukuchi, K., et al., Antiviral Res., 11:285-298 (1989)).

Cyclooxygenase (COX-1, COX-2) or prostaglandin endoperoxide H synthase (PGHS-1, PGHS-2) enzymes are widely used to measure the anti-inflammatory effects of plant products (Bayer, T., et al., Phytochemistry, 28:2373-2378 (1989); and Goda, Y., et al., Chem. Pharm. Bull., 40:2452-2457 (1992)). COX enzymes are the pharmacological target sites for nonsteroidal anti-inflammatory drugs (Humes, J. L., et al., Proc. Natl. Acad. Sci. U.S.A., 78:2053-2056 (1981); and Rome, L. H., et al., Proc. Natl. Acad. Sci. U.S.A., 72:4863-4865 (1975)). Two isozymes of cyclooxygenase involved in prostaglandin synthesis are cyclooxygenase-1 (COX-1) and cyclooxygenase-2 (COX-2) (Hemler, M., et al., J. Biol. Chem., 25:251, 5575-5579 (1976)). It is hypothesized that selective COX-2 inhibitors are mainly responsible for anti-inflammatory activity (Masferrer, J. L., et al., Proc. Natl. Acad. Sci. U.S.A., 91:3228-3232 (1994)). Flavonoids are now being investigated as anti-inflammatory substances, as well as for their structural features for cyclooxygenase (COX) inhibition activity.

Although cinnamon is generally safe and non-toxic even at high doses, it may induce allergic reactions in individuals who are sensitive to cinnamon or Peruvian balsa. It is not recommended during pregnancy and lactation. There are no known interactions with other drugs.

What is needed are novel and reproducible cinnamon extracts that combine purified essential oil, purified polyphenolics with high flavonol glycosides and flavonoids, and polysaccharide chemical constituent fractions that can be produced with standardized and reliable amounts of these synergistically acting, physiologically and medically beneficial cinnamon chemical constituents. Williamson E M. Phtomedicine 8:401-409, 2001.

Extractions

Essential Oil Fraction

Cinnamon bark is rich in essential oil and provides various kinds of oils depending on the part of plant used. It was reported that there is 1-2% essential oil by % mass weight in cinnamon bark. The main component of cinnamon bark oil is the aromatic aldehyde-3-phenyl-2(E)-propenal, also called cinnamaldehyde (about 60% in essential oil by mass weight).

Cinnamon bark was used as feedstock for current research. Supercritical carbon dioxide extraction and fractionation technology has been chosen for extraction due to its well-known benefit on processing of lipid soluble chemicals. Its usefulness for extraction is due to the combination of gas-like mass transfer properties and liquid-like solvating characteristics with diffusion coefficients greater than those of liquid solvents. The extracted essential oil constituents were assayed using gas chromatography-mass spectroscopy. Total 71 compounds have been identified from cinnamon bark oil extracted by supercritical CO2. Besides major cinnamaldehyde's congeners, such as benzaldehyde (P1), cinnamaldehyde (P10 and P14), cinnamyl alcohol (P16), trans-cinnamic acid (P23), cinnamyl acetate (P25), other minor compounds including: 4 monoterpenes, 16 sesquiterpenes, 9 fatty acids and their derivatives, and 6 steroids (P64 and P67 P71) have also been identified. Fatty acids and steroids have not previously been reported in cinnamon oil.

It was found that supercritical CO2 is an excellent tool to purify and profile essential oil fractions. The extraction yield of these fractions varies depending on processing temperature, pressure, and solvent/feed ratio. The highest extraction yield was 1.76% by mass weight at temperature of 80° C. and pressure of 100-500 bar with a solvent/feed ratio of 114. In crude extracted cinnamon bark essential oil, cinnamaldehyde accounts for 58%-69% by mass weight of the purified fractions. It was found that up to 20% of steroid compounds in extracts in extract fractions can only be extracted at low temperatures of about 40 C. High purity of cinnamaldehyde's congeners (greater than 90%) can be obtained at high temperatures of 60-90° C. and low pressures of about 100 bar. High pressure and temperature are better for processing fatty acid compounds and the highest purity can be up to ˜10% in extract fractions.

The crude extracted cinnamon bark essential oil can also be fractioned by multistage stage processing by increase processing pressure sequentially at fixed temperature. The results are shown in Table 2. It was found that the major compounds cinnamaldehyde congeners can be profiled between 67.1-93.1%. Other minor compounds, as sesquiterpene can be profiled between 1.1-2.7%; fatty acid can be profiled between 0.9-9.9%; steroids can only be extracted at temperature of 40° C. and can be profiled between 0.0-20.3% by % mass weight of the fraction (relative abundance). The highest purity of cinnamaldehyde can be up to 91.13%, which is 76 times greater than that found of that in cinnamon bark feedstock.

TABLE 2
Cinnamon essential oil compounds profile in extracts obtained at different conditions

| | T = 40° C. | | | | T = 60° C. | | | T = 80° C. | | |
|---|---|---|---|---|---|---|---|---|---|---|
| Compounds | Stage 1 | Stage 2 | Stage 3 | Stage 4 | Stage 1 | Stage 2 | Stage 3 | Stage 1 | Stage 2 | Stage 3 |
| Cinnamaldehyde | 67.3 | 88.0 | 83.3 | 67.1 | 93.1 | 86.2 | 74.7 | 90.7 | 88.9 | 74.1 |
| congeners | | | | | | | | | | |
| Sesquiterpene | 1.4 | 1.5 | 2.1 | 2.1 | 2.7 | 1.7 | 2.0 | 1.1 | 1.1 | 3.5 |
| Fatty acids and | 0.9 | 2.5 | 6.6 | 9.9 | 0.9 | 5.9 | 8.6 | 1.0 | 4.1 | 7.8 |
| derivatives | | | | | | | | | | |
| Steroids | 20.3 | 5.2 | 0.3 | 0.8 | 0.0 | 0.0 | 0.0 | 0.0 | 0.0 | 0.0 |

Phenolic Acid Fraction

Antioxidant activity of cinnamon is related to the phenolic acid chemical constituent content. Specific antioxidant phytochemicals that have been identified in cinnamon include the following phenolic acids: epicatechin, camphene, engenol, gamma-terpinene, phenol, salicylic acid and tannins. More recently, scientists at the US department of agriculture found one type of flavonoid, type-A procyanidin, extracted by water that mimics the effect of insulin. This compound potentiates insulin action in isolated adipocytes. In-vivo studies also showed that cinnamon water extracts improve insulin actions via increasing glucose uptake, in part through enhancing the insulin-signaling pathway in skeletal muscle. The object of this section of the present invention is to purify phenolic acids by removing tannin acids. The phenolic acids of interests due to their hypoglycemic activity are the proanthocyanidins. The proanthocyanindins are mixtures of oligomers and polymers containing (+)-catechin and/or (−)-epicatechin units linked through C4-C8 and/or C4-C6 bonds (B-type). These flavan-3-ol can also be doubled linked by a C4-C8 bond and an additional ether bond between C7-C2 bond (A-type). Due to lack of a commercial available HPLC reference standard, the Folin-Ciocalteu method was used to analysis total phenolic acid content and the protein-precipitable phenolics method to analysis total tannin acid content. Individual phenolic acids in the total phenolic acids were identified and semi-quantified by Direct Analysis in Real-time (DART) mass spectrometry.

In the cinnamon bark feedstock, there is about 4.87% total phenolic acid, in which about 2.27% is nontannin phenolic acids and about 2.61% is tannin acids. Total phenolic acid extraction conditions were optimized by studying the effect of different solvents, temperatures, PH values, and multistage processing. It was found that aqueous ethanol (25-75% ethanol) were optimum extraction solvents. The highest extraction yield were found at about 17.6% by using 25% ethanol as the extraction solvent at a temperature of 40° C. using two stage of processing at solvent feed ratio of 10 and 5 respectively. No pH value change needed during processing.

Sephadex LH-20 dextran beads were found to be an excellent media to separate nontannin phenolic acids from tannin acids. The results are shown in Table 3. It was found that tannin acid has been remarkably removed and nontannin phenolic acid has been purified to up to 100% (44 fold of that in feedstock).

TABLE 3
Cinnamon phenolics weight percentage changing
during sephadex LH-20 processing.

| | | | | | | B1- | B1- |
|---|---|---|---|---|---|---|---|
| | Feed | B1-F3 | B1-F4 | B1-F5 | B1-F6 | F7 | F8 |
| | Weight % | | | | | | |
| Nontannin | 2.27 | 29.5 | 66.4 | 87.8 | 91.1 | 100 | 93.8 |
| phenolic | | | | | | | |
| acids | | | | | | | |
| Tannin acids | 2.61 | 0 | 0 | 0 | 0 | 0 | 0 |

Polysaccharide Fraction

Cinnamon polysaccharide-glycoprotein fraction were obtained by water extraction and 80% ethanol precipitation. The yield of purified cinnamon polysaccharide-glycoprotein fractions was about 3.5%. The purity of cinnamon polysaccharide was 0.29-0.47 g dextran equivalent/g polysaccharide. (Dextran was used as reference standard because no cinnamon polysaccharide standards are available). The average molecular weight of cinnamon polysaccharide was ˜2500 KDa. AccuTOF-DART mass spectrometry was also used to characterize cinnamon polysaccharide, the results are shown in FIGS. 6 and 7.

Extractions Relative to Natural Cinnamon Species

This disclosure comprises extractions of isolated and purified fractions of essential oils (or essential oil sub-fractions), polyphenolic acids, and polysaccharides from one or more cinnamon species. These individual fractions can be combined in specific ratios (profiles) to provide beneficial combinations and can provide reliable or reproducible extract products that are not found in currently know extract products. For example, an essential oil fraction or sub-fraction from one species may be combined with an essential oil fraction or sub-fraction from the same or different species or with a polyphenolic acid fraction from the same or different species, and that combination may or may not be combined with a polysaccharide fraction from the same or different species of cinnamon.

Extractions of the disclosure may also be defined in terms of concentrations relative to those found in natural cinnamon species. Embodiments also comprise extractions wherein one or more of the fractions, including essential oils, polyphenolic acids, or polysaccharides, are found in a concentration that is greater than that found in native cinnamon species plant material. Embodiments also comprise extractions wherein one or more of the fractions, including essential oils, polyphenolics, or polysaccharides, are found in a concentration that is less than that found in native cinnamon species. Known amounts of the bio-active chemical constituent fractions of the cinnamon species (Table 1) are used as an example of the disclosure. For example, extractions of the disclosure comprise fractions wherein the concentration of essential oils is from 0.001 to 50 times the concentration of native cinnamon species, and/or compositions where the concentration of desired polyphenolic acids is from 0.001 to 50 times the concentration of native cinnamon species, and/or compositions where the concentration of water soluble-ethanol insoluble polysaccharides is from 0.001 to 20 times the concentration of native cinnamon species.

Extractions of the disclosure comprise fractions wherein the concentration of essential oils is from 0.01 to 50 times the concentration of native cinnamon species, and/or compositions wherein the concentration of desired polyphenolic acids is from 0.01 to 50 times the concentration of native cinnamon species, and/or compositions wherein the concentration of polysaccharides is from 0.01 to 20 times the concentration of native cinnamon species. Furthermore, extractions of the disclosure comprise sub-fractions of the essential oil chemical constituents having at least one or more of chemical compounds present in the native plant material essential oil that is in amount greater or less than that found in native cinnamon plant material essential oil chemical constituents. For example, the chemical compound, trans-cinnamaldehyde, may have it's concentration increased in an essential oil sub-fraction to 80% by % mass weight of the sub-fraction from its concentration of 60% by % mass weight of the total essential oil chemical constituents in the native cinnamon plant material. In contrast, trans-cinnamaldehyde may have it's concentration reduced in an essential oil sub-fraction to about 6% by % mass weight of the sub-fraction from it's concentration of about 60% by % mass weight of the total essential oil chemical constituents in the native plant material, a 10 fold decrease in concentration. Extractions of the disclosure comprise fractions wherein the concentration of specific chemical compounds in such novel essential oil sub-fractions is either increase by about 1.1 to about 10 times or decreased by about 0.1 to about 10 times that concentration found in the native cinnamon essential oil chemical constituents.

Additional embodiments comprise extractions comprising altered profiles (ratio distribution) of the chemical constituents of the cinnamon species in relation to that found in the native plant material or to currently available cinnamon species extract products. For example, the essential oil fraction may be increased or decreased in relation to the polyphenolic acids and/or polysaccharide concentrations. Similarly, the polyphenolic acids or polysaccharides may be increased or decreased in relation to the other extract constituent fractions to permit novel constituent chemical profile extractions for specific biological effects. By combining the isolated and purified fractions of one or more of essential oils, polyphenolics and/or polysaccharides, extractions may be made that provide novel combinations of essential oils.

Methods of the disclosure comprise providing novel cinnamon extractions for treatment and prevention of human disorders. For example, a novel cinnamon species extraction for treatment of type 2 diabetes mellitus may have an increased polyphenolic fraction concentration and reduced essential oil and polysaccharide fraction concentrations, by % weight, than that found in the cinnamon species native plant material or conventional known extraction products. A novel cinnamon species extraction for anti-oxidant, anti-blood vessel damage, and ischemic cerebrovascular disease may have an increased essential oil and polyphenolic acid fraction and a reduced polysaccharide fraction, by % weight, than that found in the native cinnamon species plant material or conventional known extraction products. Another example of a novel cinnamon species extraction, for treatment of allergic disorders comprises a fraction having an increased polyphenolic fraction concentration, an increased polysaccharide fraction, and a reduced essential oil fraction than that found in native cinnamon species plant material or known conventional extraction products.

Methods of Extraction

The following methods as taught may be used individually or in combination with the disclosed method or methods known to those skilled in the art. The starting material for extraction is plant material from one or more cinnamon species. The plant material may be the any portion of the plant, though the bark is the most preferred starting material.

The cinnamon species plant material may undergo pre-extraction steps to render the material into any particular form, and any form that is useful for extraction is contemplated by the disclosure. Such pre-extraction steps include, but are not limited to, that wherein the material is chopped, minced, shredded, ground, pulverized, cut, or torn, and the starting material, prior to pre-extraction steps, is dried or fresh plant material. A preferred pre-extraction step comprises grinding and/or pulverizing the cinnamon species bark material into a fine powder. The starting material or material after the pre-extraction steps can be dried or have moisture added to it. Once the cinnamon species plant material is in a form for extraction, methods of extraction are contemplated by the disclosure.

Methods of extraction of the disclosure comprise processes disclosed herein. In general, methods of the disclosure comprise, in part, methods wherein cinnamon species plant material is extracted using supercritical fluid extraction (SFE) with carbon dioxide as the solvent (SCCO₂) that is followed by one or more solvent extraction steps, such as, but not limited to, water, hydroalcoholic, and affinity polymer absorbent extraction processes. Additional other methods contemplated for the disclosure comprise extraction of cinnamon species plant material using other organic solvents, refrigerant chemicals, compressible gases, sonification, pressure liquid extraction, high speed counter current chromatography, molecular imprinted polymers, and other known extraction methods. Such techniques are known to those skilled in the art. In one aspect, extractions of the disclosure may be prepared by a method comprising the steps depicted schematically in FIGS. 1-5.

The disclosure includes processes for concentrating (purifying) and profiling the essential oil and other lipid soluble compounds from cinnamon plant material using SCCO₂ technology. The disclosure includes the fractionation of the lipid soluble chemical constituents of cinnamon into, for example, an essential oil fraction of high purity (high essential oil chemical constituent concentration). Moreover, the disclosure includes a SCCO₂ process wherein the individual chemical constituents within an extraction fraction may have their chemical constituent ratios or profiles altered. For example, SCCO₂ fractional separation of the chemical constituents within an essential oil fraction permits the preferential extraction of certain essential oil compounds relative to the other essential oil compounds such that an essential oil extract sub-fraction can be produced with a concentration of certain compounds greater than the concentration of other compounds. Extraction of the essential oil chemical constituents of the cinnamon species with SCCO₂ as taught in the disclosure eliminates the use of toxic organic solvents and provides simultaneous fractionation of the extracts. Carbon dioxide is a natural and safe biological product and an ingredient in many foods and beverages.

In performing the previously described extraction methods, it was found that greater than 80% yield by mass weight of the essential oil chemical constituents having greater than 95% purity of the essential oil chemical constituents in the original dried cinnamon bark feedstock of the cinnamon species can be extracted in the essential oil SCCO₂ extract fraction (Step 1A). Using the methods as taught in Step 1B (SCCO₂ Extraction and Fractionation Processes), the essential oil yield was reduced due to the fractionation of the essential oil chemical constituents into highly purified (>90%) essential oil sub-fractions. In addition, the SCCO₂ extraction and fractionation process as taught in this disclosure permits the ratios (profiles) of the individual chemical compounds comprising the essential oil chemical constituent fraction to be altered such that unique essential oil sub-fraction profiles can be created for particular medicinal purposes. For example, the concentration of the steroid essential oil chemical constituents may be increased while simultaneous reducing the concentration of the fatty acid compounds or visa versa.

Using the methods as taught in Step 2 of this disclosure, a water soluble fraction is achieved with a 4.8% mass weight yield from the original cinnamon species feedstock having a 26.0% concentration of total phenolic acids, a yield of about 10% mass weight of the phenolic acid chemical constituents found in the native cinnamon bark feedstock. However, this water solvent extract does contain valuable water soluble-ethanol insoluble polysaccharide chemical constituents. In addition, this extraction step achieves about 100% yield of the water soluble, ethanol insoluble polysaccharides found in the native cinnamon species plant material. The polysaccharide concentration in this water-soluble extraction fraction is about 27% by % dry mass weight in this water soluble extract fraction. Using 95% ethanol to precipitate the polysaccharides, a purified polysaccharide fraction may be collected from this water leaching extract. The yield of the polysaccharide fraction is about 1.3% by % mass weight based on the cinnamon rhizome feedstock. Based on a colormetric analytical method using dextran as reference standards, a purity of >95% cinnamon polysaccharides compounds may be obtained.

Using the methods as taught in Step 3 of this disclosure, a hydroalcoholic leaching fraction is achieved with a 17.6% yield from the original cinnamon species feedstock having a 64% concentration of phenolic acids, about ⅓ of the phenolic acids being non-bioactive tannins. This further equates to about a 90% yield of the phenolic acid related chemical constituents found in the native cinnamon species plant material.

Using the methods as taught in Step 4 of this disclosure (Affinity Adsorbent Extraction Processes or Process Chromatography), polyphenolic acid fractions with purities of greater than 95% by % dry mass of the extraction fraction with less than 0.1% tannins by % mass weight may be obtained. It is possible to extract about 77% of the non-tannin polyphenolic acids from the hydroalcoholic leaching extract feedstock. This equates to a 69% yield of the polyphenolic acid chemical constituents found in the native cinnamon species plant material. Based on the average degree of polymerization, the purified polyphenolic fractions are largely made of the beneficial bioactive polyphenolic oligomers.

Furthermore, it is possible to profile the polyphenolic chemical constituents of the purified polyphenolic fractions. For example, purified polyphenolic sub-fractions may be obtained containing a high concentration of polyphenolic trimers or tetramers. Such novel purified polyphenolic sub-fractions may have great value for specific medical conditions.

Finally, the methods as taught in the disclosure permit the purification (concentration) of the cinnamon species essential oil chemical constituent fractions, novel polyphenolic fractions or sub-fractions, and a novel polysaccharide fraction to be as high as 99%% by mass weight of the desired chemical constituents in the essential oil fractions, as high as 97% by mass weight in the polyphenolic phenolic fraction, and as high as 98% by mass weight in the polysaccharide fraction. The specific extraction environments, rates of extraction, solvents, and extraction technology used depend on the starting chemical constituent profile of the source material and the level of purification desired in the final extraction products. Specific methods as taught in the disclosure can be readily determined by those skilled in the art using no more than routine experimentation typical for adjusting a process to account for sample variations in the attributes of starting materials that is processed to an output material that has specific attributes. For example, in a particular lot of cinnamon species plant material, the initial concentrations of the essential oil chemical constituents, the polyphenolic acids, and the polysaccharides are determined using methods known to those skilled in the art as taught in the disclosure. One skilled in the art can determine the amount of change from the initial concentration of the essential oil chemical constituents, for instance, to the predetermined amounts or distribution (profile) of essential oil chemical constituents for the final extraction product using the extraction methods, as disclosed herein, to reach the desired concentration and/or chemical profile in the final cinnamon species extraction product.

A schematic diagram of the methods of extraction of the biologically active chemical constituents of cinnamon is illustrated in FIGS. 1-5. The extraction process is typically, but not limited to, 4 steps.

Step 1: Supercritical Fluid Carbon Dioxide Extraction of Cinnamon Essential Oil

Due to the hydrophobic nature of the essential oil, non-polar solvents, including, but not limited to SCCO₂, hexane, petroleum ether, and ethyl acetate may be used for this extraction process. Since some of the components of the essential oil are volatile, steam distillation may also be used as an extraction process.

A generalized description of the extraction of the essential oil chemical constituents from the bark of the cinnamon species using SCCO₂ is diagrammed in FIG. 2-Step 2A and 2B. The feedstock 10 is dried ground cinnamon bark (about 140 mesh). The extraction solvent 210 is pure carbon dioxide. Ethanol may be used as a co-solvent. The feedstock is loaded into a SFE extraction vessel 20. After purge and leak testing, the process comprises liquefied CO₂ flowing from a storage vessel through a cooler to a CO₂ pump. The CO₂ is compressed to the desired pressure and flows through the feedstock in the extraction vessel where the pressure and temperature are maintained at the desired level. The pressures for extraction range from about 60 bar to 800 bar and the temperature ranges from about 35° C. to about 90° C. The SCCO₂ extractions taught herein are preferably performed at pressures of at least 100 bar and a temperature of at least 35° C., and more preferably at a pressure of about 60 bar to 500 bar and at a temperature of about 40° C. to about 80° C. The time for extraction for a single stage of extraction range from about 30 minutes to about 2.5 hours, to about 1 hour. The solvent to feed ratio is typically about 60 to 1 for each of the SCCO₂ extractions. The CO₂ is recycled. The extracted, purified, and profiled essential oil chemical constituents 30 are then collected a collector or separator, saved in a light protective glass bottle, and stored in a dark refrigerator at 4° C. The cinnamon feedstock 10 material may be extracted in a one step process (FIG. 2, Step 2A) wherein the resulting extracted and purified cinnamon essential oil fraction 30 is collected in a one collector SFE or SCCO₂ system 20 or in multiple stages (FIG. 2, Step 2B) wherein the extracted purified and profiled cinnamon essential oil sub-fractions 50, 60, 70, 80 are separately and sequentially collected in a one collector SFE system 20. Alternatively, as in a fractional SFE system, the SCCO₂ extracted cinnamon feedstock material may be segregated into collector vessels (separators) such that within each collector there is a differing relative percentage essential oil chemical constituent fraction (profile) in each of the purified essential oil sub-fractions collected. The residue (remainder) 40 is collected, saved and used for further processing to obtain purified fractions of the cinnamon species phenolic acids and polysaccharides. An embodiment of the disclosure comprises extracting the cinnamon species feedstock material using multi-stage SCCO₂ extraction at a pressure of 60 bar to 500 bar and at a temperature between 35° C. and 90° C. and collecting the extracted cinnamon material after each stage. A second embodiment of the disclosure comprises extracting the cinnamon species feedstock material using fractionation SCCO₂ extraction at pressures of 60 bar to 500 bar and at a temperature between 35° C. and 90° C. and collecting the extracted cinnamon material in differing collector vessels at predetermined conditions (pressure, temperature, and density) and determined intervals (time). The resulting extracted cinnamon purified essential oil sub-fractions from each of the multi-stage extractors or in differing collector vessels (fractional system) can be retrieved and used independently or can be combined to form one or more cinnamon essential oil fractions comprising a predetermined essential oil chemical constituent concentration that is higher or lower than that found in the native plant material or in conventional cinnamon extraction products. Typically, the total yield of the essential oil fraction from cinnamon species using a single step maximal SCCO₂ extraction is about 0.4 to about 1.8% (>85% of the essential oil chemical constituents) by % weight having an essential oil chemical constituent purity of greater than 95% by mass weight of the extract. The results of such extraction processes are found below and in Table 4. The procedure can be found in Example 1.

TABLE 4
HPLC analysis of single stage SFE cinnamon essential oil extraction.

| | | Density | | | CND | CND yield |
|---|---|---|---|---|---|---|
| T (° C.) | P (bar) | (g/cc) | S/F | Yield (%) | purity (%) | (%) |
| 40 | 80 | 0.293 | 57 | 0.46 | 69.1 | 0.32 |
| 40 | 100 | 0.64 | 57 | 0.87 | 60.2 | 0.53 |
| 40 | 120 | 0.723 | 57 | 0.87 | 61.5 | 0.53 |
| 40 | 300 | 0.915 | 57 | 1.27 | 58.0 | 0.74 |
| 60 | 80 | 0.195 | 38 | 0.34 | 65.4 | 0.22 |
| 60 | 100 | 0.297 | 38 | 0.34 | 68.1 | 0.23 |
| 60 | 120 | 0.448 | 38 | 0.43 | 67.1 | 0.29 |
| 60 | 300 | 0.834 | 38 | 1.14 | 58.7 | 0.67 |
| 80 | 100 | 0.226 | 19 | 0.49 | 68.0 | 0.33 |
| 80 | 300 | 0.751 | 19 | 1.14 | 59.6 | 0.68 |

These results demonstrate the effect of pressure on the kinetics of extraction. Higher extraction pressures result in the system reaching equilibrium at shorter times with less amount of CO₂ consumed. The total extraction yield increases with increasing extraction pressure due to the density increase associated with pressure increase. Interestingly, a lower pressures such as 100-300 bar, the lower the temperature, the higher the yield again related to a higher density. At higher pressures such as 300-500 bar, temperature has far less effect of the extraction yield. Although a higher yield and greater efficiency of extraction may be achieved with pressures greater than 200 bar, 95% purity of the essential oil chemical constituents can be achieved with pressures less than 300 bar and temperatures of about 40-80° C.

In the experiment range investigated, it can be clearly noted that there is a competition effect between temperature and density. This aspect is well defined and documented in the literature, where an increase in pressure, at constant temperature, leads to an increase in the yield due to the enhancement in the solvency power of the supercritical and near critical fluid. An increase in temperature promotes an enhancement in vapor pressure of the compounds favoring the extraction. Additionally, the increase in diffusion coefficient and the decrease in solvent viscosity also help the compounds extraction from the herbaceous porous matrix as the temperature is increased to higher value. On the other hand, an increase in temperature, at constant system pressure, leads to a decrease in the solvent density.

Seventy-one compounds were separated and identified in cinnamon bark essential oil using GC-MS analysis. By comparing the mass spectra data of sample with the data in the scientific literature, cinnamaldehyde, coumarin, and cinnamyl acetate were identified. (Tables 3 and 4) In addition to cinnamaldehyde and it's cogeners such as benzaldhyde (P1), cinnamaldehyde (P10 & P14), cinnamyl alcohol (P16), trans-cinnamic acid (P23), and cinnamyl acetate (P25), 4 monoterpenes (P6, P8, and P9), 16 sesquiterpenes (P20-22, P26, P29, P31-2, P35-42, and P46), and 9 fatty acids and fatty acid derivatives were identified. Other minor aromatic and aliphatic compounds were also present. Of the compounds identified, SFE was able to extract fatty acids and steroid compounds that had not previously been identified in cinnamon essential oil. These compounds make up about 90% of the essential oil chemical constituents by % mass weight. Cinnamaldehyde is the major chemical constituent of the cinnamon essential oil at about 70-91% by % mass weight. A greater number of compounds were identified from extractions under the conditions of 40° C. and 120 bar with higher purity of about 100% than at SFE extraction conditions of higher temperatures and pressures. Cinnamaldehyde purity of greater than 90% mass weight was accomplished with SFE temperatures of 60° C. and 100 bar with a loss of steroid compounds and lower fatty acid and sesquiterpene purity. Steroid compounds can only be extracted a low temperature of 40° C. At a SFE temperature of 40° C. and 80 bar, the steroid compound chemical constituent purity was as high as 20% mass weight. In contrast, higher SFE temperatures (60-80° C.) and pressures (500 bar) favor the extraction of the fatty acid compounds. These data indicate that SCCO₂ has the ability to profile the chemical constituents of cinnamon essential oil.

TABLE 5
Compounds Identified in Cinnamon Essential Oil Fraction

| | Ret | | | | | |
|---|---|---|---|---|---|---|
| Peak | time | | | | | |
| ID | (min) | Compound | CAS# | Formula | Mw | structure |
| P1 | 7.2 | Benzaldehyde | 100-52-7 | C7H6O | 106 | 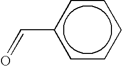 |
| P2 | 9.9 | Benzeneacetaldehyde | 122-78-1 | C8H8O | 120 | 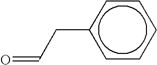 |
| P3 | 10.6 | Acetophenone | 98-86-2 | C8H8O | 120 | 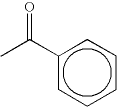 |
| P4 | 10.8 | Benzoylcarboxaldehyde | 1074-12-0 | C8H6O2 | 134 | 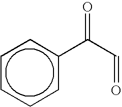 |
| P5 | 14.1 | Benzenepropanal | 104-53-0 | C9H10O | 134 | 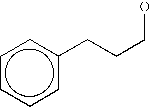 |
| P6 | 14.3 | Borneol | 507-70-0 | C10H18O | 154 | 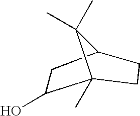 |
| P7 | 14.6 | Benzofuran, 2-methyl- | 4265-25-2 | C9H8O | 132 | 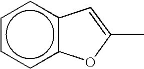 |
| P8 | 14.7 | 1-Terpinen-4-ol | 562-74-3 | C10H18O | 154 | 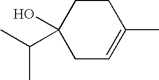 |
| P9 | 15.2 | α-Terpieol | 10482-56-1 | C10H18O | 154 | 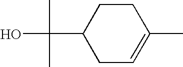 |
| P10 | 16.1 | Cinnamylaldehyde | 104-55-2 | C9H8O | 132 | 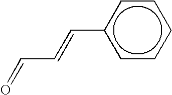 |
| P11 | 16.5 | Benzenepropanol | 122-97-4 | C9H12O | 136 | 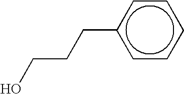 |
| P12 | 16.8 | Benzoylformic acid | 611-73-4 | C8H6O3 | 150 | 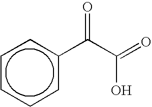 |
| P13 | 17.5 | Benzene, 1,3-bis(1,1- dimethylethyl)- | 1014-60-4 | C14H22 | 190 | 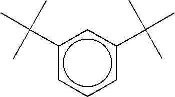 |
| P14 | 18.4 | Cinnamaldehyde, (E)- | 14371-10-9 | C9H8O | 132 | 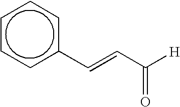 |
| P15 | 18.8 | Acetic acid, bornyl ester | 92618-89-8 | C12H20O2 | 196 | 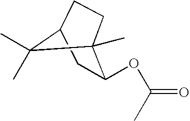 |
| P16 | 19.5 | Cinnamyl alcohol | 104-54-1 | C9H10O | 134 | 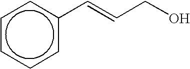 |
| P17 | 20.0 | 2,4-Decadienal | 2363-88-4 | C10H16O | 152 | 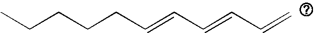 |
| P18 | 20.5 | 2,4-dimethyl-1-heptanol | 18450-74-3 | C9H20O | 144 | 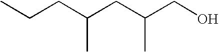 |
| P19 | 22.0 | Megastigam- 4,6(E),8(E)-triene | 51468-86-1 | C13H20 | 176 | 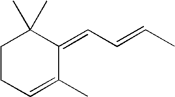 |
| P20 | 23.6 | Copaene | 3856-25-5 | C15H24 | 204 | 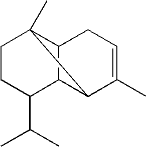 |
| P21 | 26.3 | 1,3,6,10- Dodecatetraene, 3,7,11-trimethyl-, (Z,E)- | 26560-14-5 | C15H24 | 204 | 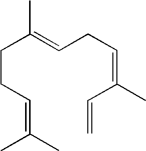 |
| P22 | 26.6 | Beta-caryophyllene | 87-44-5 | C15H24 | 204 | 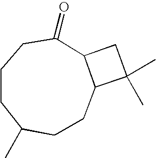 |
| P23 | 26.9 | trans-Cinnamic acid | 140-10-3 | C9H8O2 | 148 | 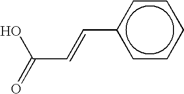 |
| P24 | 27.4 | Coumarin | 91-64-5 | C9H6O2 | 146 | 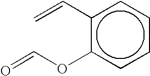 |
| P25 | 28.5 | Cinnamyl acetate | 103-54-8 | C11H12O2 | 176 | 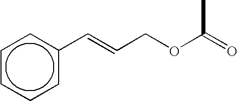 |
| P26 | 34.0 | α-Muurolene | 31983-22-9 | C15H24 | 204 | 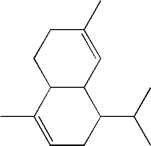 |
| P27 | 34.2 | 3-(phenylmethoxy)-1- propanol | 4799-68-2 | C10H14O2 | 166 | 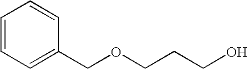 |
| P28 | 35.0 | Phenol, 3,5-bis(1,1- dimethylethyl)- | 1138-52-9 | C14H22O | 206 | 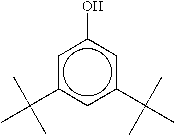 |
| P29 | 35.7 | (−)-Calamenene | 483-77-2 | C15H22 | 202 | 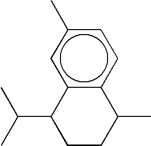 |
| P30 | 35.9 | Cinnamaldehyde, o- methoxy- | 1504-74-1 | C10H10O2 | 162 | 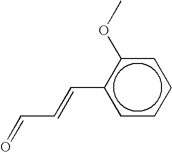 |
| P31 | 36.4 | 1,2,3,4,4A,7- hexahydro-1,6- dimethyl-4-(1- methylethyl)- naphthalene | 16728-99-7 | C15H24 | 204 | 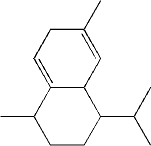 |
| P32 | 39.3 | β-Caryophyllene epoxide | 1139-30-6 | C15H24O | 220 | 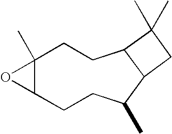 |
| P33 | 40.7 | unknown 1 | | | | |
| P34 | 41.5 | Benzaldehyde, 4- propyl- | 28785-06-0 | C10H12O | 148 | 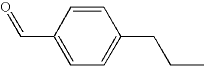 |
| P35 | 41.8 | Cubenol | 21284-22-0 | C15H26O | 222 | 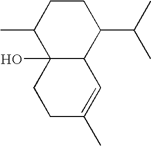 |
| P36 | 42.1 | .alpha.-Cadinol | 481-34-5 | C15H26O | 222 | 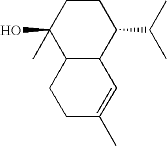 |
| P37 | 42.4 | delta-cardinol | 36563-42-8 | C15H26O | 222 | 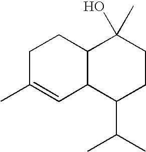 |
| P38 | 42.6 | α-muurolol | 19435-97-3 | C15H26O | 222 | 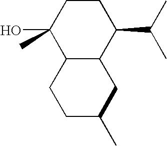 |
| P39 | 42.9 | .tau.-muurolol | 19912-62-0 | C15H26O | 222 | 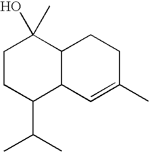 |
| P40 | 43.1 | Germacrene D | 23986-74-5 | C15H24 | 204 | 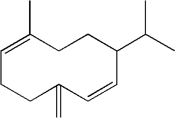 |
| P41 | 43.3 | .alpha.-Cubebene | 17699-14-8 | C15H24 | 204 | 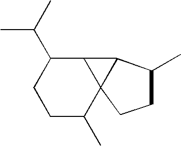 |
| P42 | 43.7 | 1H- Cycloprop[e]azulene, decahydro-1,1,4,7- tetramethyl-,[1aR- (1a.alpha.,4.beta.,4a.be- ta.,7.beta.,7a.beta.,7b.al- pha.)]- | 28580-43-0 | C15H26 | 206 | 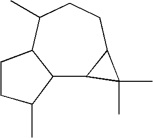 |
| P43 | 43.8 | Naphthalene, 1,6- dimethyl-4-(1- methylethyl)- | 483-78-3 | C15H18 | 198 | 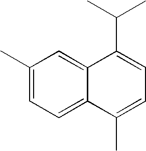 |
| P44 | | unknown | | | | |
| P45 | 44.7 | 2-Propenoic acid, tridecyl ester | 4/8/3076 | C16H30O2 | 254 | 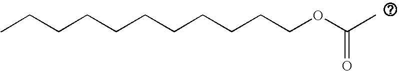 |
| P46 | 47.6 | 1,2,3,4,4A,7- hexahydro-1,6- dimethyl-4-(1- methylethyl)- naphthalene | 16728-99-7 | C15H24 | 204 | 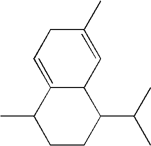 |
| P47 | 48.6 | Propanoic acid, 3- hydroxy-3-phenyl-,t- butyl ester | 5397-27-3 | C13H18O3 | 222 | 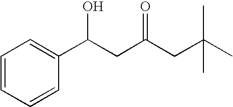 |
| P48 | 49.7 | 2-Dodecanol, 2-methyl- | 1653-37-8 | C13H28O | 200 | 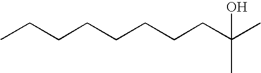 |
| P49 | 51.1 | 1-Hexadecanol | 36653-82-4 | C16H34O | 242 | 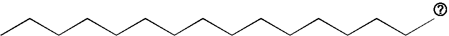 |
| P50 | 52.4 | pentadecanoic acid, methyl ester | 7132-64-1 | C16H32O2 | 256 | 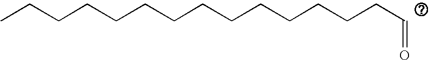 |
| P51 | 52.6 | 1,19-Eicosadiene | 14811-95-1 | C20H38 | 278 | 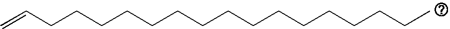 |
| P52 | 53.4 | n-Hexadecanoic acid | 57-10-3 | C16H32O2 | 256 | 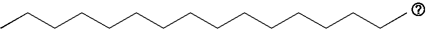 |
| P53 | 56.0 | Oleyl Alcohol | 143-28-2 | C18H36O | 268 | 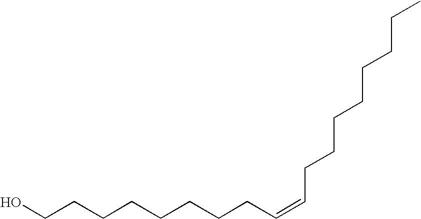 |
| P54 | 56.6 | 1-Nonadecanol | 1454-84-8 | C19H40O | 284 | 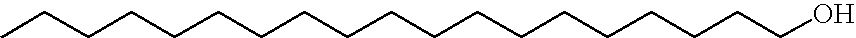 |
| P55 | 57.9 | Ethanol, 2-(9,12- octadecadienyloxy)-, (Z,Z)- | 17367-08-7 | C20H38O2 | 310 | 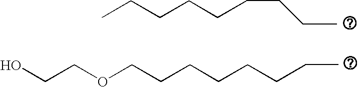 |
| P56 | 58.0 | 9-Octadecenoic acid (Z)- | 112-80-1 | C18H34O2 | 282 | 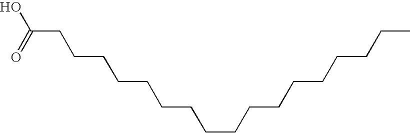 |
| P57 | 58.2 | unknowns | | | | unknowns |
| P58 | 58.6 | Eicosanoic acid | 506-30-9 | C20H40O2 | 312 | 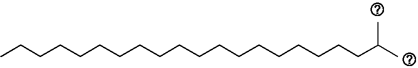 |
| P59 | 59.1 | Hexadecanoic acid, butyl ester | 111-06-8 | C20H40O2 | 312 | 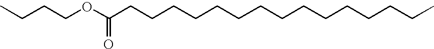 |
| P60 | 63.8 | Octadecanoic acid, butyl ester | 123-95-5 | C22H44O2 | 340 | 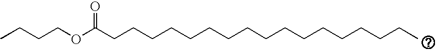 |
| P61 | 64.1 | Heneicosane | 629-94-7 | C12H44 | 296 | 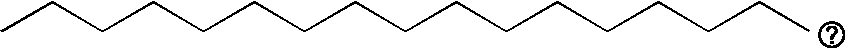 |
| P62 | 64.9 | Benzenepropanoic acid, 10- oxotricyclo[4.2.1.1(2,5)]deca-3,7-dienyl ester | 0-00-0 | C19H18O3 | 294 | |
| P63 | 66.0 | Cyclopentanemethanol, 2-nitro-.alpha.-(2- phenylethenyl)-, [1.alpha.(S@),2.alpha.]- | 103130-01-4 | C14H17NO3 | 247 | 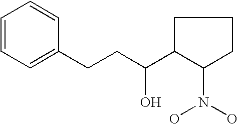 |
| P64 | | 7,22-Ergostadienol | | C28H46O | 398 | 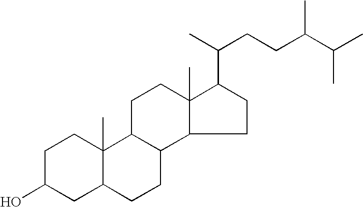 |
| P65 | 67.7 | unknown | | | | |
| P66 | 68.6 | 1,2- Benzenedicarboxylic acid, diisooctyl ester | 27554-26-3 | C24H38O4 | 390 | 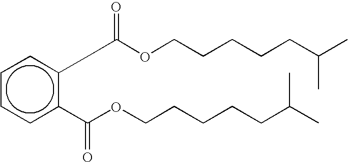 |
| P67 | 68.7 | .beta.-Sitosterol | 83-46-5 | C29H50O | 414 | 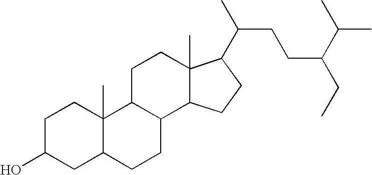 |
| P68 | 70.6 | Ergosta-7,22-dien-3-ol, (3.beta.,22E)- | 17608-76-3 | C28H46O | 398 | 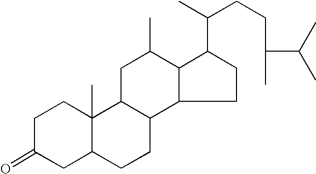 |
| P69 | 72.6 | 4,4,6a,6b,8a,11,11,14b- Octamethyl- 1,4,4a,5,6,6a,6b,7,8,8a, 9,10,11,12,12a,14,14a, 14b-octadecahydro-2H- picen-3-one | | C30H48O | 424 | 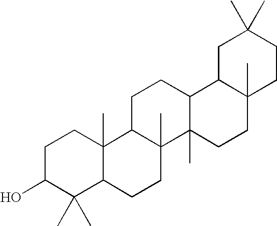 |
| P70 | 74.4 | Ergosta-7,22-dien-3-ol, (3.beta.,5. alpha., 22E)- | 11/4/2645 | C28H46O | 398 | 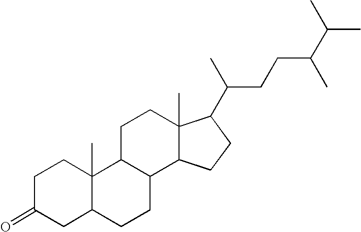 |
| P71 | 76.9 | Chondrillasterol | 481-17-4 | C29H48O | 412 | 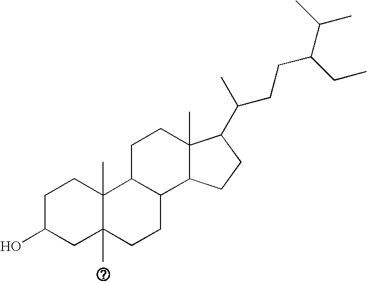 |

TABLE 6
GC-MS analysis peak area, peak area percentage and calculated weight percentage of
cinnamon bark essential oil extracted at different conditions.

| | | T = 40° C., P = 300 bar | | | T = 80° C., P = 100 bar | | | T = 80° C., P = 300 bar | | |
|---|---|---|---|---|---|---|---|---|---|---|
| | Ret. | | peak | | | peak | | | peak | |
| Peak | time | | area | | | area | | | area | |
| No. | (min) | peak area | % | Weight % | peak area | % | Weight % | peak area | % | Weight % |
| P1 | 7.201 | 107275 | 0.03 | 0.03 | 3572872 | 0.57 | 0.57 | 161232 | 0.04 | 0.04 |
| P2 | 9.866 | | | | 239555 | 0.04 | 0.04 | | | |
| P3 | 10.649 | | | | 94664 | 0.02 | 0.02 | | | |
| P4 | 10.822 | | | | 275862 | 0.04 | 0.04 | | | |
| P5 | 14.054 | 29489 | 0.01 | 0.01 | | | | 160992 | 0.04 | 0.04 |
| P6 | 14.282 | 358823 | 0.11 | 0.11 | | | | 386330 | 0.09 | 0.09 |
| P7 | 14.563 | 374620 | 0.12 | 0.12 | | | | 432491 | 0.1 | 0.1 |
| P8 | 14.692 | 400042 | 0.12 | 0.12 | | | | 413562 | 0.1 | 0.1 |
| P9 | 15.153 | 683952 | 0.21 | 0.21 | 200566 | 0.03 | 0.03 | 890990 | 0.21 | 0.21 |
| P10 | 16.112 | 1672873 | 0.51 | 0.51 | 4210512 | 0.67 | 0.67 | 1090882 | 0.26 | 0.26 |
| P11 | 16.528 | 98413 | 0.03 | 0.03 | 305036 | 0.05 | 0.05 | 264483 | 0.06 | 0.06 |
| P12 | 16.848 | | | | 138317 | 0.02 | 0.02 | | | |
| P13 | 17.471 | 314610 | 0.1 | 0.1 | 451375 | 0.07 | 0.07 | 244719 | 0.06 | 0.06 |
| P14 | 18.388 | 228756437 | 70.29 | 70.29 | 560967679 | 89.76 | 89.76 | 358267571 | 86.02 | 86.02 |
| P15 | 18.798 | 804095 | 0.25 | 0.25 | | | | 680662 | 0.16 | 0.16 |
| P16 | 19.499 | 1329676 | 0.41 | 0.41 | 3744998 | 0.6 | 0.6 | 3918089 | 0.94 | 0.94 |
| P17 | 20.038 | 189718 | 0.06 | 0.06 | 215682 | 0.03 | 0.03 | 156726 | 0.04 | 0.04 |
| P18 | 20.494 | 86462 | 0.03 | 0.03 | | | | 60887 | 0.01 | 0.01 |
| P19 | 21.954 | 176284 | 0.05 | 0.05 | 165025 | 0.03 | 0.03 | 205784 | 0.05 | 0.05 |
| P20 | 23.566 | 2473168 | 0.76 | 0.76 | | | | 1468434 | 0.35 | 0.35 |
| P21 | 26.287 | 137651 | 0.04 | 0.04 | | | | 206073 | 0.05 | 0.05 |
| P22 | 26.592 | 367241 | 0.11 | 0.11 | | | | 802082 | 0.19 | 0.19 |
| P23 | 26.908 | | | | | | | 339296 | 0.08 | 0.08 |
| P24 | 27.432 | 15068316 | 4.63 | 4.63 | 23526861 | 3.77 | 3.77 | 25045782 | 6.01 | 6.01 |
| P25 | 28.466 | 3071028 | 0.94 | 0.94 | 12812414 | 2.05 | 2.05 | 4063398 | 0.98 | 0.98 |
| P26 | 33.953 | 387927 | 0.12 | 0.12 | 281914 | 0.05 | 0.05 | 553811 | 0.13 | 0.13 |
| P27 | 34.237 | | | | 281619 | 0.05 | 0.05 | | | |
| P28 | 35.035 | 111608 | 0.03 | 0.03 | | | | 85138 | 0.02 | 0.02 |
| P29 | 35.674 | 158637 | 0.05 | 0.05 | 551592 | 0.09 | 0.09 | 290965 | 0.07 | 0.07 |
| P30 | 35.881 | 119357 | 0.04 | 0.04 | 422786 | 0.07 | 0.07 | 486137 | 0.12 | 0.12 |
| P31 | 36.356 | 72181 | 0.02 | 0.02 | | | | 118059 | 0.03 | 0.03 |
| P32 | 39.334 | 335593 | 0.1 | 0.1 | 1272069 | 0.2 | 0.2 | 460812 | 0.11 | 0.11 |
| P33 | 40.650 | 49352 | 0.02 | 0.02 | | | | | | |
| P34 | 41.464 | 83100 | 0.03 | 0.03 | | | | 246325 | 0.06 | 0.06 |
| P35 | 41.750 | 176194 | 0.05 | 0.05 | 924151 | 0.15 | 0.15 | 354941 | 0.09 | 0.09 |
| P36 | 42.087 | 181715 | 0.06 | 0.06 | 160919 | 0.03 | 0.03 | 245345 | 0.06 | 0.06 |
| P37 | 42.390 | 93210 | 0.03 | 0.03 | 629354 | 0.1 | 0.1 | | | |
| P38 | 42.586 | | | | 311094 | 0.05 | 0.05 | 86504 | 0.02 | 0.02 |
| P39 | 42.927 | | | | 331623 | 0.05 | 0.05 | 117555 | 0.03 | 0.03 |
| P40 | 43.140 | 165961 | 0.05 | 0.05 | 795638 | 0.13 | 0.13 | 469680 | 0.11 | 0.11 |
| P41 | 43.339 | | | | 279943 | 0.04 | 0.04 | 154116 | 0.04 | 0.04 |
| P42 | 43.672 | | | | 144713 | 0.02 | 0.02 | 85123 | 0.02 | 0.02 |
| P43 | 43.789 | | | | 125052 | 0.02 | 0.02 | | | |
| P44 | | | | | | | | 78201 | 0.02 | 0.02 |
| P45 | 44.739 | 153149 | 0.05 | 0.05 | 861436 | 0.14 | 0.14 | 205244 | 0.05 | 0.05 |
| P46 | 47.604 | | | | 232837 | 0.04 | 0.04 | | | |
| P47 | 48.648 | 86340 | 0.03 | 0.03 | 201161 | 0.03 | 0.03 | 158427 | 0.04 | 0.04 |
| P48 | 49.657 | 100070 | 0.03 | 0.03 | | | | 104638 | 0.03 | 0.03 |
| P49 | 51.066 | 104471 | 0.03 | 0.03 | 819892 | 0.13 | 0.13 | 494453 | 0.12 | 0.12 |
| P50 | 52.420 | | | | 106293 | 0.02 | 0.02 | | | |
| P51 | 52.609 | | | | 102916 | 0.02 | 0.02 | | | |
| P52 | 53.430 | 132985 | 0.04 | 0.04 | 930693 | 0.15 | 0.15 | 1910179 | 0.46 | 0.46 |
| P53 | 55.979 | | | | 460792 | 0.07 | 0.07 | 455416 | 0.11 | 0.11 |
| P54 | 56.611 | | | | 287130 | 0.05 | 0.05 | 360772 | 0.09 | 0.09 |
| P55 | 57.895 | | | | | | | 1058073 | 0.25 | 0.25 |
| P56 | 57.997 | | | | | | | 1451917 | 0.35 | 0.35 |
| P57 | 58.195 | 189737 | 0.06 | 0.06 | 123292 | 0.02 | 0.02 | 1178268 | 0.28 | 0.28 |
| P58 | 58.550 | | | | | | | 678103 | 0.16 | 0.16 |
| P59 | 59.112 | 932215 | 0.29 | 0.29 | 850903 | 0.14 | 0.14 | 999986 | 0.24 | 0.24 |
| P60 | 63.761 | 1876786 | 0.58 | 0.58 | | | | 1438157 | 0.35 | 0.35 |
| P61 | 64.109 | | | | | | | 146342 | 0.04 | 0.04 |
| P62 | 64.927 | | | | | | | 327089 | 0.08 | 0.08 |
| P63 | 66.005 | | | | | | | 619225 | 0.15 | 0.15 |
| P64 | 67.234 | 9979848 | 3.07 | 3.07 | | | | | | |
| P65 | 67.746 | | | | | | | 68066 | 0.02 | 0.02 |
| P66 | 68.642 | | | | | | | 152882 | 0.04 | 0.04 |
| P67 | 68.651 | 6629886 | 2.04 | 2.04 | | | | | | |
| P68 | 70.645 | 16769518 | 5.15 | 5.15 | | | | | | |
| P69 | 72.590 | 10386507 | 3.19 | 3.19 | | | | | | |
| P70 | 74.399 | 10804061 | 3.32 | 3.32 | | | | | | |
| P71 | 76.879 | 686165 | 2.67 | 2.67 | | | | | | |

| | | | | | | | | | |
|---|---|---|---|---|---|---|---|---|---|
| total | 325266746.0 | 100.0 | 100.0 | 622411230.0 | 99.6 | 99.6 | 414900414.0 | 99.6 | 99.6 |
| cinna- | 234937289.0 | 72.2 | 72.2 | 585308475.0 | 93.7 | 93.7 | 367840468.0 | 88.3 | 88.3 |
| maldehyde | | | | | | | | | |
| congeners | | | | | | | | | |
| aromaric | 251223142.0 | 77.2 | 77.2 | 611370763.0 | 97.8 | 97.8 | 395724862.0 | 95.0 | 95.0 |
| compounds | | | | | | | | | |
| nomoterpene | 1442817.0 | 0.4 | 0.4 | 200566.0 | 0.0 | 0.0 | 1690882.0 | 0.4 | 0.4 |
| sesquiterpene | 4390841.0 | 1.3 | 1.3 | 5364255.0 | 0.9 | 0.9 | 5122535.0 | 1.2 | 1.2 |
| fatty acid and | 3489413.0 | 1.1 | 1.1 | 4543347.0 | 0.7 | 0.7 | 10481548.0 | 2.5 | 2.5 |
| its derivatives | | | | | | | | | |
| steroids | 63255985.0 | 19.4 | 19.4 | 0.0 | 0.0 | 0.0 | 0.0 | 0.0 | 0.0 |

Note:
weight % were calculated by:
Weight % = (weight of each compound/total weight of extracts) × 100 where weight of each compound = peak area percentage × total weight of extracts.

Step 2. Water Leaching Process and Polysaccharide Precipitation

The polysaccharide extract fraction of the chemical constituents of cinnamon species has been defined in the scientific literature as the “water soluble, ethanol insoluble extraction fraction”. A generalized description of the extraction of the polysaccharide fraction from extracts of cinnamon species using water solvent leaching and ethanol precipitation processes is diagrammed in FIG. 3-Step 2. The feedstock 10 or 40 is native ground cinnamon species plant material or the solid residue from the SFE extraction process of Step 1. This feedstock is leaching extracted in two stages. The solvent is distilled water 220. In this method, the cinnamon species feedstock 10 or 40 and the extraction solvent 220 are loaded into an extraction vessel 100, 110 and heated and stirred. It may be heated to 100° C., to about 80° C., or to about 80-90° C. The extraction is carried out for about 1-5 hours, for about 2-4 hours, or for about 2 hours. The two stage extraction solutions 300+320 are combined and the slurry is filtered 120, centrifuged 130, and the supernatant collected and evaporated 140 to remove water until an about 8-fold increase in concentration of the chemicals in solution 330. Anhydrous ethanol 230 is then used to reconstitute the original volume of solution making the final ethanol concentration at 95%. A large precipitate 150 is observed. The solution is centrifuged 160, decanted 170 and the supernatant residue 340 may be saved for further processing. The precipitate product 350 is the purified polysaccharide fraction that may be analyzed for polysaccharides using the colormetric method by using Dextran 5,000-410,000 molecular weight as reference standards. The actual procedure can be found in Example 3. The purity of the extracted polysaccharide fraction using 3 different molecular weight dextran as standards is about 29, 35, and 47%, respectively, with a total yield of 1.3% by % mass weight of the original native cinnamon bark feedstock. Combining the purity measures of the 3 dextran standards indicates a very high level of purity of greater than 95%. Moreover, AccuTOF-DART mass spectrometry (see Exemplification section) was used to further profile the molecular weights of the compounds comprising the purified polysaccharide fraction. The actual procedure can found in the Exemplification section.

Step 3. Hydroalcoholic Leaching Process for Extraction of Crude Polyphenolic Acid Fraction

In one aspect, the disclosure comprises extraction and concentration of the bio-active polyphenolic acid chemical constituents. A generalized description of this step is diagrammed in FIG. 4-STEP 3. This Step 2 extraction process is a solvent leaching process. The feedstock for this extraction is either cinnamon species ground dry bark material 10 or the residue 40 or 330+340 from the Step 1 SCCO₂ extraction of the essential oil chemical constituents or the Step 2 polysaccharide extraction-precipitation, respectively. The extraction solvent 240 is aqueous ethanol. The extraction solvent may be 10-95% aqueous alcohol, 25% aqueous ethanol is preferred. In this method, the cinnamon feedstock material and the extraction solvent are loaded into an extraction vessel 400 that is heated and stirred. It may be heated to 100° C., to about 90° C., to about 80° C., to about 70° C., to about 60° C. or to about 30-50° C. The extraction is carried out for about 1-10 hours, for about 1-5 hours, for about 2 hours. The resultant extract solution is filtered 410 and centrifuged 420. The filtrate (supernatant) 500, 520, 540 is collected as product, measured for volume and solid content dry mass after evaporation of the solvent. The extraction residue material 530 may be retained and saved for further processing or discarded. The extraction may be repeated as many times as is necessary or desired. It may be repeated 2 or more times, 3 or more times, 4 or more times, etc. For example, FIG. 1-STEP 2 shows a three stage process, where the second stage and the third stage use the same methods and conditions

Interestingly, residual cinnamaldehyde was extracted with this hydroalcoholic leaching extraction process indicating that not all of the essential oil chemical constituents were extracted with relatively exhaustive extraction using the above SFE conditions. Moreover, a significant amount of tannins were extracted making up greater than 20% of the extraction product. Moreover, a two stage hydroalcoholic leaching process is preferred to achieve a high extraction yield of polyphenolics (about 18% by mass weight based on the raw feedstock material) with a total phenolic acid concentration of about 64% by mass weight and a tannin acid concentration of about 20% by mass weight. In order to develop a purified polyphenolic fraction containing a high concentration of bioactive polyphenolics, an additional processing step (Step 4) is required to remove the tannins from the crude Step 3 polyphenolic fraction.

Step 4. Affinity Adsorbent Polyphenolic Extraction and Purification Process

The beneficial bioactive polyphenolic acids are proanthocyanidins. Proanthocyanidin are known as condensed tannins. They are ubiquitous and present as the second most abundant natural plant polyphenolics after lignins. Dubois M et al. Analytical Chem 28:350-356, 1956. The proanthocyanidins are mixtures of oligomers and polymers consisting of (+)-catechin and/or (−)-epicatechin units linked mainly through C4-C8 and/or C4-C6 bonds (B-type). These flavan-3-ol can be double linked by a C4-C8 bond and an additional ether bond between O7-C2 (A type). The molecular weight of proanthocyanidins expressed as degree of polymerization (DPn) is one of the most important properties. As defined in the scientific literature, DP1 is a monomer, DP2-10 are oligomers, and DP>10 are polymers, respectively.

In the biomedical literature regarding cinnamon polyphenolics (see above), DP 4-5 (oligomers) exhibit the medically beneficial biological activity. Therefore, in Step 4 processing, tannin removal and proanthocyanidin extraction and purification has been studied by tracking total phenolic acid concentration and DPn in each step of processing.

As taught herein, a purified polyphenolic acid fraction extract from cinnamon and related species may be obtained by contacting a hydroalcoholic extract of cinnamon feedstock with a solid affinity polymer adsorbent resin so as to adsorb the polyphenolic acids contained in the hydro-alcoholic extract onto the affinity adsorbent. The bound chemical constituents are subsequently eluted by the methods taught herein. Prior to eluting the polyphenolic acid fraction chemical constituents, the affinity adsorbent with the desired chemical constituents adsorbed thereon may be separated from the remainder of the extract in any convenient manner, preferably, the process of contacting with the adsorbent and the separation is effected by passing the aqueous extract through an extraction column or bed of the adsorbent material.

A variety of affinity adsorbents can be utilized to purify the phenolic acid chemical constituents of cinnamon species, such as, but not limited to Sephadex LH-20 (Sigma Aldrich Co.), “Amberlite XAD-2” (Rohm & Hass), “Duolite S-30” (Diamond Alkai Co.), “SP207” (Mitsubishi Chemical), ADS-5 (Nankai University, Tianjin, China), ADS-17 (Nankai University, Tianjin, China), Dialon HP 20 (Mitsubishi, Japan), and Amberlite XAD7 HP (Rohm & Hass).

Sephadex LH020 is preferably used for process chromatography due to the high affinity for the polyphenolic acid chemical constituents of and its ability to separate tannin polyphenolics from non-tannin polyphenolics. The tannin polyphenolics adsorb to Sephadex LH-20 in alcohol. In contrast non-tannin polyphenoics can be eluted from the resin beads using alcohol whereas the tannins remain adsorb on the beads. The tannins can then be eluted later with aqueous acetone. This method permits the separation of the tannin polyphenolic from the desired non-tannin polyphenolics of cinnamon. Thus, different elution solvents can be used for the separation of the polyphenolic compounds and purification of the non-tannin bioactive cinnamon polyphenolics. Using the Folin-Ciocalteu method and the protein-precipitable phenolic method, the tannin and non-tannin polyphenolic concentrations can be measured in the crude extraction fraction and the elution fractions.

Although various eluants may be employed to recover the non-tannin polyphenolic acid chemical constituents from the adsorbent, in one aspect of the disclosure, the eluant comprises low molecular weight alcohols, including, but not limited to, methanol, ethanol, or propanol. In a second aspect, the eluant comprises low molecular alcohol in an admixture with water. In another aspect, the eluant comprises low molecular weight alcohol, a second organic solvent, and water.

Although various eluants may be employed to recover the tannin polyphenolic acid chemical constituents, in one aspect of the disclosure, the eluant comprises aqueous acetone.

Preferably, the cinnamon species feedstock has undergone a one or more preliminary purification process such as, but not limited to, the processes described in Step 1 and 3 prior to contacting the aqueous phenolic acid chemical constituent containing extract with the affinity adsorbent material.

Using affinity adsorbents as taught in the disclosure results in highly purified bioactive polyphenolic oligomers (DP2-10) acid chemical constituents of the cinnamon species that are remarkably free of other chemical constituents which are normally present in natural plant material or in available commercial extraction products. For example, the processes taught in the disclosure can result in purified polyphenolic acid extracts that contain total phenolic acid chemical constituents in excess of 95% by dry mass weight containing only trace tannin polyphenolics.

The extraction and purification of the bioactive polyphenolic acids from the bark of the cinnamon species using polymer affinity adsorbent resin beads is diagrammed in FIG. 1-Step 4. The feedstock for this extraction process may be the aqueous ethanol solution containing the phenolic acids from Step 3 hydroalcoholic Leaching Extraction 500+/−520+/−540. The appropriate weight of adsorbent resin beads (22 mg of polyphenolic acids per gm of adsorbent resin) is washed (soaked) with 4-5 BV of 95% ethanol 250 prior to being packed into a column 620. The polyphenolic acid containing aqueous solution 500+520 is concentrated using evaporation to 1% of its original volume. Then, absolute ethanol 260 is added to the concentrated sample sufficient to increase the volume 20 times, dissolving the polyphenolics in a 95% ethanol solution. This solution is centrifuged 640 to remove any insoluble material and the supernatant collected as the loading sample 550. The loading sample 550 is loaded onto the column 650. Once the column is fully loaded, the column is eluted 660 with 95% ethanol 270 at a flow rate of 2-3 BV/hour to elute the bioactive non-tannin polyphenolics in an isocratic fashion from the affinity adsorbent column. The eluant 700 is collected in 1 BV fractions. The polyphenolic fractions are each tested by UV spectrophotometer at 280 nm (polyphenolic acid wave length absorbance) until the absorbance is not longer detected in the fraction samples at which time the elution is discontinued. Generally 7-10 BV of 95% ethanol are required to elute the non-tannin polyphenolics from the column (about 3-4 hours). The eluted column 670 is washed 680 with 3 BV of 70% aqueous acetone 280 eluting the tannin polyphenolics adsorbed on the resin beads at a flow rate of 5 BV/hr (3 hours). The eluted tannin polyphenolic washing 710 is discarded 730. The washed column 730 is then washed with 4-5 95% ethanol 250 at a flow rate of 5 BV/hr to remove any remaining chemicals in the column preparing the washed column for further process chromatography 740. The washing 720 is discarded 730. The elution fraction volumes 700 may be collected about every 1 BV and these samples are analyzed total polyphenolics (Folin-Ciocalteu method), tannin polyphenolics (Protein-precipitation Method, DPn (Thiolytic degradation HPLC) and tested for solids content and purity.

The oligomeric and polymeric proanthocyanidin polyphenolic compounds are eluted on a wide retention window (retention times 12-30 min) causing baseline deviation and difficulty with precise integration of the chromatographic peaks when calculating the catechin and epicatechin concentration. This HPLC behavior has been verified for most proanthocyanidins in the scientific literature. However, after thiolysis, the HPLC chromatograms clearly show evidence of the improvement of chromatographic resolution. With tholysis, the proanthocyanidins are converted into monomeric units yielding well-resolved peaks on the HPLC chromatograms. Benzylthioethers result from the extension unit of proanthocyanidin structures according to the scientific literature (see Guyot 2001). The DPn can be calculated by the total area of P1, P2, P3, and P4 and the total area of catechin and epicatechin.

Sephadex LH-20 has been shown to be an efficient affinity adsorbent for the separation of tannin from nontannin polyphenolic compounds in cinnamon hydroalcoholic extracts. Combining elution fractions F2-F8 about 77.4% the non-tannin polyphenolic chemical constituents can be recovered with only 0.2% of the tannins being recovered in this combined extraction fraction. The yield of combining elution fractions F2-F8 is 21.5% by mass weight of the loading solution and 3.78% by mass weight based on the raw cinnamon feedstock. The non-tannin polyphenolic purity is 65% by mass dry weight which is 3 times higher than the crude polyphenolic extraction product of Step 3. Moreover, a purity of greater than 95% by % mass weight can be found by combining elution fractions F6-F8.

The average degree of polymerization (DPn) demonstrates the size of the polyphenolic oligomer in each elution fraction. In the crude extract (loading solution), the degree of polymerization was 6.9 due to the presence of the large tannin polyphenolic polymers. In the polyphenolic elution fractions, essentially no tannin polyphenolics were found. Therefore, the purified polyphenolic elution fractions are made up largely of polyphenolic oligomers, a mixture of dimers-DPn=2; trimers-DPn=3; tetamers-DPn=4; etc.). As shown in Table 5, more trimers were eluted in elution fractions F3-F5 and more tetramers were eluted in elution fractions F6-F8. The range of DPn in the elution fractions was from 2.7 to 4.2 confirming that these fractions contain a high level purity of the beneficial bioactive proanthocyanidin polyphenolic chemical constituents of cinnamon. Furthermore, by combining different elution fractions, different extraction products having different purities of the nontannin polyphenolic and yields can be achieved as demonstrated in Tables 7 and 8.

TABLE 7
Analysis of 95% ethanol elutions of polyphenolic fractions from Sephadex LH-20
process chromatography.

| | | Weight (mg) | | | | Purity (%) | | |
|---|---|---|---|---|---|---|---|---|
| | | | Total | | | Non | | |
| | Yield | Total | phenolic | Tannin | Nontannin | Tannin | tannin | Average |
| Name | (%) | solid | acid | acid | acid | acid | acid | DPn |
| Loading | | 132.1 | 61.2 | 32.8 | 28.5 | 21.6 | 24.8 | 6.9 |
| Elution F2 | 37.1 | 49.0 | 3.7 | 0.1 | 3.6 | 7.1 | 0.1 | 3.6 |
| Elution F3 | 7.4 | 9.8 | 2.9 | 0.0 | 2.9 | 29.5 | 0.0 | 2.7 |
| Elution F4 | 5.2 | 6.8 | 4.5 | 0.0 | 4.5 | 66.4 | 0.0 | 3.6 |
| Elution F5 | 3.2 | 4.2 | 3.7 | 0.0 | 3.7 | 87.8 | 0.0 | 3.1 |
| Elution F6 | 2.3 | 3.1 | 2.9 | 0.0 | 2.9 | 91.1 | 0.0 | 4.0 |
| Elution F7 | 2.1 | 2.8 | 2.9 | 0.0 | 2.9 | 100.0 | 0.0 | 4.2 |
| Elution F8 | 1.2 | 1.6 | 1.6 | 0.0 | 1.6 | 93.8 | 0.0 | 4.2 |
| Combine | 5.7 | 7.5 | 7.3 | 0.0 | 7.3 | 97.2 | 0.0 | 4.1 ⊥ 0.1 |
| F6-F8 | | | | | | | | |
| Combine | 21.5 | 28.4 | 18.5 | 0.0 | 18.5 | 65.1 | 0.0 | 3.6 ⊥ 0.6 |
| F2-F8 | | | | | | | | |
| Recovery | | 58.5 | 36.1 | 0.2 | 77.4 | | | |
| (%) | | | | | | | | |

* Elution 1 was not tabulated because there was chemical constituents, only solvent.

TABLE 8
Results of yield and purity
of different nontannin polyphenolic elution fractions.

| | Total | Total | Total phenolic | | |
|---|---|---|---|---|---|
| | solid | phenolic | acid purity | Yield based on | |
| Fractions | (mg) | acid (mg)* | (%) | feedstock (%) | DPn |
| F2-F7 | 28.4 | 18.5 | 65.1 | 3.8 | 3.6 |
| F3-F7 | 18.6 | 15.6 | 83.7 | 2.5 | 3.8 |
| F4-F7 | 11.8 | 11.0 | 93.8 | 1.6 | 3.9 |
| F5-F7 | 7.5 | 7.3 | 97.2 | 1.0 | 4.1 |
| F6-F7 | 4.4 | 4.4 | 100.0 | 0.6 | 4.2 |

*Total phenolic acid have no measurable tannin acids in these combined fractions.

Many methods are known in the art for removal of alcohol from solution. If it is desired to keep the alcohol for recycling, the alcohol can be removed from the solutions, after extraction, by distillation under normal or reduced atmospheric pressures. The alcohol can be reused. Furthermore, there are also many methods known in the art for removal of water from solutions, either aqueous solutions or solutions from which alcohol was removed. Such methods include, but not limited to, spray drying the aqueous solutions onto a suitable carrier such as, but not limited to, magnesium carbonate or maltodextrin, or alternatively, the liquid can be taken to dryness by freeze drying or refractive window drying.

Food and Medicaments

As a form of foods of the present invention, there may be formulated to any optional forms, for example, a granule state, a grain state, a paste state, a gel state, a solid state, or a liquid state. In these forms, various kinds of substances conventionally known for those skilled in the art which have been allowed to add to foods, for example, a binder, a disintegrant, a thickener, a dispersant, a reabsorption promoting agent, a tasting agent, a buffer, a surfactant, a dissolution aid, a preservative, an emulsifier, an isotonicity agent, a stabilizer or a pH controller, etc. may be optionally contained. An amount of the elderberry extract to be added to foods is not specifically limited, and for example, it may be about 10 mg to 5 g, preferably 50 mg to 2 g per day as an amount of take-in by an adult weighing about 60 kg.

In particular, when it is utilized as foods for preservation of health, functional foods, etc., it is preferred to contain the effective ingredient of the present invention in such an amount that the predetermined effects of the present invention are shown sufficiently.

The medicaments of the present invention can be optionally prepared according to the conventionally known methods, for example, as a solid agent such as a tablet, a granule, powder, a capsule, etc., or as a liquid agent such as an injection, etc. To these medicaments, there may be formulated any materials generally used, for example, such as a binder, a disintegrant, a thickener, a dispersant, a reabsorption promoting agent, a tasting agent, a buffer, a surfactant, a dissolution aid, a preservative, an emulsifier, an isotonicity agent, a stabilizer or a pH controller.

An administration amount of the effective ingredient (cinnamon extract) in the medicaments may vary depending on a kind, an agent form, an age, a body weight or a symptom to be applied of a patient, and the like, for example, when it is administrated orally, it is administered one or several times per day for an adult weighing about 60 kg, and administered in an amount of about 10 mg to 5 g, preferably about 50 mg to 2 g per day. The effective ingredient may be one or several components of the cinnamon extract.

Methods also comprise administering such extracts more than one time per day, more than two times per day, more than three times per day and in a range from 1 to 15 times per day. Such administration may be continuously, as in every day for a period of days, weeks, months, or years, or may occur at specific times to treat or prevent specific conditions. For example, a person may be administered cinnamon species extracts at least once a day for years to enhance mental focus, cognition, and memory, or to prevent and treat type 2 diabetes mellitus, to prevent cardiovascular disease stroke, or to treat gastro-intestinal disorders, or to treat inflammatory disorders and arthritis including gout, or to treat the common cold, bacterial and fungal infections.

The foregoing description includes the best presently contemplated mode of carrying out the disclosure. This description is made for the purpose of illustrating the general principles of the disclosures and should not be taken in a limiting sense. This disclosure is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the disclosure.

All terms used herein are considered to be interpreted in their normally accepted usage by those skilled in the art. Patent and patent applications or references cited herein are all incorporated by reference in their entireties.

### EXEMPLIFICATION

Materials

Acetone (67-64-1), >99.5%, ACS reagent (179124); Acetonitrile (75-05-8), for HPLC, gradient grade ≧99.9% (GC) (000687); Hexane (110-54-3), 95+%, spectrophotometric grade (248878); Ethyl acetate (141-78-6), 99.5+%, ACS grade (319902); Ethanol, denatured with 4.8% isopropanol (02853); Ethanol (64-17-5), absolute, (02883); Methanol (67-56-1), 99.93%, ACS HPLC grade, (4391993); and Water (7732-18-5), HPLC grade, (95304). All were purchased from Sigma-Aldrich.

Formic acid (64-18-6), 50% solution (09676); Acetic acid (64-19-7), 99.7+%, ACS reagent (320099); Hydrochloric acid (7647-01-0), volumetric standard 1.0N solution in water (318949); Calcium hydroxide (7789-78-8), powder, CA 0-2 mm, 90-95% (213268); Ferric chloride anhydrous (7705-08-0), 97%, reagent grade(157740); Folin-Clocalteu phenol reagent (2N) (47641); Phenol (108-95-2) (P3653); Sulfuric acid (7664-93-9), ACS reagent, 95-97% (44719); Triethanolamine(102-71-6), triethanolamine free base (T1377); Sodium dodecyl sulfate(151-21-3), minimum 98.5% GC (L4509); all were purchased from Sigma-Aldrich. Sodium carbonate (S263-1, Lot #: 037406) was purchased from Fisher Co.

Serum albumin (9048-46-8), Albumin Bovine Fraction V powder cell culture tested (A9418); (+)-catechin hydrate (88191-48-4), purity >98% (C1251); Gallic acid (149-91-7), ACS reagent, ≧98% (HPLC); Benzylthiol (100-53-8), 99% (B25401); Trans-cinnamaldehyde (14371-10-9), 99+% purity; tannin acid (1401-55-4), powder (T0125); all were purchased from Sigma-Adrich. (−)-epicatechin 93.6% (05125-550, CAS# 490-46-0) was purchased from Chromadex. Dextran standard 5000 (00269), 50,000 (00891) and 410,000 (00895) certified according to DIN were purchased from Fluka. The structures of chemical reference standards used in the disclosure are shown below:

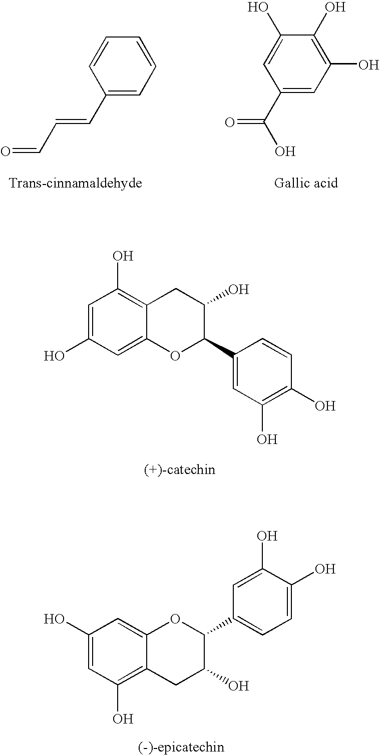

Sephadex LH-20: Sephadex™ LH-20 (Lot #: 308822, pack 167600, product #: 17-0090-01) were purchased from Ambersham Bioscience AB Uppsala Sweden. It is prepared by hydroxypropylation of sephadex G-25, a bead-formed dextran medium, and has been specifically developed for gel filtration of natural products, such as steroids, terpenoids, lipids and low molecular weight peptides, in organic solvent.

HPLC Method

Chromatographic system: Shimadzu high Performance Liquid Chromatographic LC-10AVP system equipped with LC10ADVP pump with SPD-M 10AVP photo diode array detector. The extraction products obtained were measured on a reversed phase Jupiter C18 column (250×4.6 mm I. D., 5 [symbol] , 300 Å) (Phenomenex, Part #: 00G-4053-EO, serial No: 2217520-3, Batch No.: 5243-17). The injection volume was 10 [symbol] l and the flow rate of mobile phase was 1 ml/min. The column temperature was 50° C. The mobile phase consisted of A (0.5% aqueous formic acid, v/v) and B (acetonitrile). The gradient was programmed as follows: with the first 6 minutes, A maintains at 100%, 6-10 min, solvent B increased linearly from 0% to 12%, and 10-35 min, B linearly from 12% to 21%, then 35-40 min, B linearly from 21% to 25%, then 40-50 min, B linearly to 100%.

Methanol stock solutions of 3 reference standards (catechin, epicatechin and Trans-cinnalmaldehyde) were prepared by dissolving weighted quantities of standard compounds into methanol at 1 mg/ml. The mixed reference standard solution was then diluted step by step to yield a series of solutions at final concentrations of 0.75, 0.5, 0.1, 0.05 mg/ml, respectively. All the stock solutions and working solution were used within 7 days and stored in +4° C. chiller and brought to room temperature before use. The solutions were used to identify and quantify the compounds in cinnamon. Retention times of (+)-catechin (C), (−)-epicatechin (EC), and trans-cinnamaldehyde (CAN) were about 14.02, 15.22, and 34.00 min, respectively. A linear fit ranging from 0.01 to 10 [symbol] g was found. The regression equations and correlation coefficients were as follows: (+)-catechin: peak area=465303×C ( [symbol] g) 5701.4, R²=0.9996 (N=6); (−)-epicatechin: peak area=124964×C ( [symbol] g) 215.88, R²=0.9998 (N=6); trans-cinnamaldehyde: peak area/100=69657×C ( [symbol] g)-1162.1, R²=0.9997 (N=6). HPLC results are shown in Table 9. The contents of the reference standards in each sample were calculated by interpolation from the corresponding calibration curves based on the peak area.

TABLE 9
HPLC analysis results of cinnamon standard at concentration of 1 mg/ml in methanol

| | Retention | | | | Start | Stop | |
|---|---|---|---|---|---|---|---|
| | time | Area | Height | Width | time | time | Theoretical |
| ID | (min) | (mAu · min) | (mAu) | (min) | (min) | (min) | plate¹ |
| (+)-catechin | 14.016 | 1479356 | 234337 | 0.46 | 13.83 | 14.29 | 14854 |
| (−)-epicatechin | 15.221 | 164706 | 23537 | 0.64 | 15 | 15.64 | 9050 |
| Trans- | 33.984 | 22590251 | 1029700 | 1.66 | 33.3 | 34.97 | 6706 |
| cinnalmaldehyde | | | | | | | |

¹Theoretical plates was calculated by: N = 16 × (t_{R}/w)². t_{R} is retention time and w is width of the peak, https://www.mn-net.com/web%5CMN-WEB-HPLCkatalog.nsf/WebE/GRUNDLAGEN

GC-MS Analysis

GC-MS analysis was performed using a Shimadzu GCMS-QP2010 system. The system includes high-performance gas chromatograph, direct coupled GC/MS interface, electro impact (EI) ion source with independent temperature control, quadrupole mass filter et al. The system is controlled with GCMS solution Ver. 2 software for data acquisition and post run analysis. Separation was carried out on a Agilent J&W DB-5 fused silica capillary column (30 m×0.25 mm i.d., 0.25 [symbol] m film thickness) (catalog: 1225032, serial No: U.S. Pat. No. 5,285,774H) using the following temperature program. The initial temperature was 60° C., held for 2 min, then it increased to 120° C. at rate of 4° C./min, held for 15 min, then it increased to 240° C. at rate of 4° C./min, held for 15 min with total running time of 77 minutes. The sample injection temperature was 250° C. 1 [symbol] l of the sample was injected by auto injector at splitless mode in 1 minute. The carrier gas was helium and flowrate was controlled by pressure at 60 KPa. Under such pressure, the flowrate was 1.03 ml/min and linear velocity was 37.1 cm/min. MS ion source temperature was 230° C., and GC/MS interface temperature was 250° C. MS detector was scanned between m/z of 50 and 500 at scan speed of 1000 AMU/second. Solvent cutoff temperature was 3.5 min.

Folin-Ciocalteu Method (Markar 1993) for Total Phenolic Acids

Shimazu UV-V is spectrophotometer (UV 1700 with UV probe: S/N: A1102421982LP) has been used.

Standard:

Make stock gallic acid/water solution at concentration of 1 mg/ml. Take suitable amount of gallic acid solution in test tubes, make up the volume to 0.5 ml with distilled water, add 0.25 ml of the Folin Ciocalteu reagent and then 1.25 ml of the 20 wt % sodium carbonate solution. Shake the tube well (untrasonic bath) for 40 min and record absorbance at 725 nm. The data are shown in Table 10.

TABLE 10
Preparations of calibration curve for gallic acid.

| | Gallic acid | | | | Sodium | Absorb- |
|---|---|---|---|---|---|---|
| | solution | Gallic | Distilled | Folin | carbonate | ance |
| | (0.1 mg/ml) | acid | water | reagent | solution | at 725 |
| Tube | (ml) | (μg) | (ml) | (ml) | (ml) | mm* |
| Blank | 0.00 | 0 | 0.50 | 0.25 | 1.25 | 0.000 |
| 1 | 0.02* | 2 | 0.48* | 0.25 | 1.25 | 0.111 |
| 2 | 0.04 | 4 | 0.46 | 0.25 | 1.25 | 0.226 |
| 3 | 0.06 | 6 | 0.44 | 0.25 | 1.25 | 0.324 |
| 4 | 0.08 | 8 | 0.42 | 0.25 | 1.25 | 0.464 |
| 5 | 0.1 | 10 | 0.40 | 0.25 | 1.25 | 0.608 |

*amount of gallic acid solution is depending on the absorption information

Direct Analysis in Real Time (DART) Mass Spectrometry for Polysaccharide Analysis.
Instruments: JOEL AccuTOF DART LC time of flight mass spectrometer (Joel USA, Inc., Peabody, Mass., USA). This Time of Flight (TOF) mass spectrometer technology does not require any sample preparation and yields masses with accuracies to 0.00001 mass units.
Methods: The instrument settings utilized to capture and analyze fractions are as follows: For cationic mode, the DART needle voltage is 3000 V, heating element at 250° C., Electrode 1 at 100 V, Electrode 2 at 250 V, and helium gas flow of 7.45 liters/minute (L/min). For the mass spectrometer, orifice 1 is 10 V, ring lens is 5 V, and orifice 2 is 3 V. The peaks voltage is set to 600 V in order to give resolving power starting at approximately 60 m/z, yet allowing sufficient resolution at greater mass ranges. The micro-channel plate detector (MCP) voltage is set at 2450V. Calibrations are performed each morning prior to sample introduction using a 0.5 M caffeine solution standard (Sigma-Aldrich Co., St. Louis, USA). Calibration tolerances are held to ≦5 mmu.

The samples are introduced into the DART helium plasma with sterile forceps ensuring that a maximum surface area of the sample is exposed to the helium plasma beam. To introduce the sample into the beam, a sweeping motion is employed. This motion allows the sample to be exposed repeatedly on the forward and back stroke for approximately 0.5 sec/swipe and prevented pyrolysis of the sample. This motion is repeated until an appreciable Total Ion Current (TIC) signal is observed at the detector, then the sample is removed, allowing for baseline/background normalization.

For anionic mode, the DART and AccuTOF MS are switched to negative ion mode. The needle voltage is 3000 V, heating element 250° C., Electrode 1 at 100 V, Electrode 2 at 250 V, and helium gas flow at 7.45 L/min. For the mass spectrometer, orifice 1 is 20 V, ring lens is −13 V, and orifice 2 is 5 V. The peak voltage is 200 V. The MCP voltage is set at 2450 V. Samples are introduced in the exact same manner as cationic mode. All data analysis is conducted using MassCenterMain Suite software provided with the instrument.

### Example 1

Example of Step 1A: Single Step SFE Maximal Extraction and Purification of Cinnamon Essential Oil

All SFE extractions were performed on SFT 250 (Supercritical Fluid Technologies, Inc., Newark, Del., USA) designed for pressures and temperatures up to 690 bar and 200° C., respectively. This apparatus allows simple and efficient extractions at supercritical conditions with flexibility to operate in either dynamic or static modes. This apparatus consists of mainly three modules; an oven, a pump and control, and collection module. The oven has one preheat column and one 100 ml extraction vessel. The pump module is equipped with a compressed air-driven pump with constant flow capacity of 300 ml/min. The collection module is a glass vial of 40 ml, sealed with caps and septa for the recovery of extracted products. The equipment is provided with micrometer valves and a flow meter. The extraction vessel pressure and temperature are monitored and controlled within +3 bar and −1° C.

In typical experimental examples, 30 grams of cinnamon bark powder with size above 105 [symbol] sieved by 140 mesh screen was loaded into a 100 ml extraction vessels for each experiment. Glass wool was placed at the two ends of the column to avoid any possible carry over of solid material. The oven was preheated to the desired temperature before the packed vessel was loaded. After the vessel was connected into the oven, the extraction system was tested for leakage by pressurizing the system with CO₂ (˜850 psig), and purged. The system was closed and pressurized to desired extraction pressure using the air-driven liquid pump. The system was then left for equilibrium for ˜3 min. A sampling vial (40 ml) was weighed and connected to the sampling port. The extraction was started by flowing CO₂ at a rate of ˜10 SLPM (19 g/min), which is controlled by a meter valve. The solvent/feed ratio, defined as the weight ration of total CO₂ used to the weight of loaded raw material, was calculated. During the extraction process, the extracted sample was weighed every 5 min. Extraction was presumed to be finished when the weight of the sample did not change more than 5% between two weighing measurements. The yield was defined to be the weight percentage of the essential oil extracted with respect to the initial total weight of the feedstock material loaded into the extraction vessel. A full factorial extraction design was adopted varying the temperature from 40-80° C. to 80-500 bar.

In this experimental example, the extraction conditions were set wherein the temperatures ranged from 40-80° C. and the pressures ranged from 80-500 bar. The CO₂ flow rate was 19 g/min. The results are shown in Tables 11.

TABLE 11
HPLC analysis of single stage SFE cinnamon essential oil extraction.

| | | Density | | | CND | CND yield |
|---|---|---|---|---|---|---|
| T (° C.) | P (bar) | (g/cc) | S/F | Yield (%) | purity (%) | (%) |
| 40 | 80 | 0.293 | 57 | 0.46 | 69.1 | 0.32 |
| 40 | 100 | 0.64 | 57 | 0.87 | 60.2 | 0.53 |
| 40 | 120 | 0.723 | 57 | 0.87 | 61.5 | 0.53 |
| 40 | 300 | 0.915 | 57 | 1.27 | 58.0 | 0.74 |
| 60 | 80 | 0.195 | 38 | 0.34 | 65.4 | 0.22 |
| 60 | 100 | 0.297 | 38 | 0.34 | 68.1 | 0.23 |
| 60 | 120 | 0.448 | 38 | 0.43 | 67.1 | 0.29 |
| 60 | 300 | 0.834 | 38 | 1.14 | 58.7 | 0.67 |
| 80 | 100 | 0.226 | 19 | 0.49 | 68.0 | 0.33 |
| 80 | 300 | 0.751 | 19 | 1.14 | 59.6 | 0.68 |

### Example 2

Example of Step 1B: Multi-stage SCCO₂ Fractionation of Cinnamon Essential Oil.

Multi-stage SCCO₂ extraction/fractionation was performed using a SFT 250 (Supercritical Fluid Technologies, Inc., Newark, Del., USA). In typical multi-stage extractions, 30 g ground cinnamon bark, particle size greater than 105 [symbol] m, was loaded into an extraction vessel with an internal volume of 100 ml. The extraction solution was collected in a 40 ml collector vessel connected to the exit of the extraction vessel. The flow rate of CO₂ was set at 19 g/min. The first extraction step was performed at a pressure of 80 bar and a temperature of 40° C. (CO₂ density=0.29 g/ml). This extraction step was carried out for 1 hour. The second extraction step was performed at a pressure of 100 bar and a temperature of 40° C. (CO₂ density=0.64 g/ml). The second extraction step lasted for 1 hour. The third extraction step was performed at a pressure of 120 bar and a temperature of 40° C. for 1 hour (CO₂ density=0.72 g/ml). A fourth extraction stage at a temperature of 40° C. and a pressure of 300 bar (CO₂ density=0.92 g/ml) was then performed for 1 hour. Multi-stage extractions using three stages at 60 C and 80° C. were also performed. The analytical results including are shown in Table 12 that can be compared with the crude extract and multi-stage GC-MS data under the same SFE conditions.

TABLE 12
Multiple stage SFE extraction yield of cinnamon essential oil.

| | | | Density | | Yield |
|---|---|---|---|---|---|
| stage | T (° C.) | P (bar) | (g/cc) | S/F | (%) |
| 1 | 40 | 80 | 0.293 | 38 | 0.55 |
| 2 | 40 | 100 | 0.64 | 38 | 0.55 |
| 3 | 40 | 120 | 0.723 | 38 | 0.24 |
| 4 | 40 | 300 | 0.915 | 38 | 0.26 |
| 1 | 60 | 100 | 0.297 | 38 | 0.60 |
| 2 | 60 | 300 | 0.835 | 38 | 0.35 |
| 3 | 60 | 500 | 0.938 | 38 | 0.32 |
| 1 | 80 | 100 | 0.227 | 38 | 0.75 |
| 2 | 80 | 300 | 0.751 | 38 | 0.86 |
| 3 | 80 | 500 | 0.88 | 38 | 0.14 |

The total yield of multi-stage extractions at 40, 60, and 80° C. was about 1.6%, 1.3%, and 1.8% by mass weight based on original feedstock, respectively, by summing up the yield from each stage. These yields were higher than the yields in the single stage crude extractions due to a higher solvent-feed ratio that was used in the multi-stage processing. Otherwise, the data are consistent. As is apparent from the data, the concentrations of the chemical constituent chemical compounds such as trans-cinnamaldehyde can be changed in these sub-fraction extraction products confirming the ability of SFE to profile the chemical constituents of cinnamon essential oil.

TABLE 13
Cinnamon essential oil compounds profile in extracts obtained at different conditions.

| | T = 40° C. | | | | T = 60° C. | | | T = 80° C. | | |
|---|---|---|---|---|---|---|---|---|---|---|
| Compounds | Stage 1 | Stage 2 | Stage 3 | Stage 4 | Stage 1 | Stage 2 | Stage 3 | Stage 1 | Stage 2 | Stage 3 |
| Cinnamaldehyde | 67.3 | 88.0 | 83.3 | 67.1 | 93.1 | 86.2 | 74.7 | 90.7 | 88.9 | 74.1 |
| congeners | | | | | | | | | | |
| Sesquiterpenes | 1.4 | 1.5 | 2.1 | 2.1 | 2.7 | 1.7 | 2.0 | 1.1 | 1.1 | 3.5 |
| Fatty acids | 0.9 | 2.5 | 6.6 | 9.9 | 0.9 | 5.9 | 8.6 | 1.0 | 4.1 | 7.8 |
| and derivatives | | | | | | | | | | |
| Steroids | 20.3 | 5.2 | 0.3 | 0.8 | 0.0 | 0.0 | 0.0 | 0.0 | 0.0 | 0.0 |

### Example 3

Example of Step 2 Polysaccharide Fraction Extraction

A typical experimental example of solvent extraction and precipitation of the water soluble, ethanol insoluble purified polysaccharide fraction chemical constituents of cinnamon species is as follows: 20 gm of the solid residue from the SFE extraction at 60° C. and 300 bar was extracted using 400 ml of distilled water for two hours at 85° C. in two stages. The two extraction solutions were combined and the slurry was filtered using Fisherbrand P4 filter paper (pore size 4-8 [symbol] m) and centrifuged at 2,000 rpm for 20 minutes. The supernatant was collected. Rotary evaporation was used to concentrate the clear supernatant extract solution from 800 ml to 80 ml. Then, 1520 ml of anhydrous ethanol was added to make up a final ethanol concentration of 95%. The solution was allowed to sit for 30 min and a precipitate was observed. The extraction solution was centrifuged at 2,000 rpm for 20 minutes and the supernatant decanted and either saved for further processing or discarded. Mass balance was performed before and after precipitation to calculate the yield of polysaccharides. The precipitate was collected and dried in an oven at 50° C. for 12 hours. The dried polysaccharide fraction was weighed and dissolved in water for analysis of polysaccharide purity with the colormetric method, using dextran as reference standards. Moreover, AccuTOF-DART mass spectrometry was used to further characterize the polysaccharide fraction. The results are shown in FIGS. 6 and 7 and Tables 14 and 15.

TABLE 14
Precipitated polysaccharide fraction analysis by water
leaching and using 95% ethanol precipitate.

| | SFE 60° C. and |
|---|---|
| | 300 bar residue |
| Feedstock (g) | 20 |
| Water leaching yield (%) | 4.8 |
| Leaching extracts before precipitate (g) | 0.96 |
| Leaching extracts after precipitate (g) | 0.71 |
| Precipitate (pcp) (g) | 0.25 |
| Precipitate yield (%) | 1.3 |
| Total phenolic acid before precipitate (g) | 0.25 |
| Total phenolic acid after precipitate (g) | 0.26 |
| Dextran 5K (mg/mg pcp) | 0.47 |
| Dextran 50 K (mg/mg pcp) | 0.35 |
| Dextran 410 K (mg/mg pcp) | 0.29 |

TABLE 15
DART analysis polysaccharide from cinnamon.

| | |
|---|---|
| Positive Ion | Negative Ion |

| (m + H)/z | Relative Intensity | (m − H)/z | Relative Intensity |
|---|---|---|---|
| 84.28124 | 99.425442 | 75.01006 | 137.56585 |
| 86.25373 | 81.720883 | 76.98839 | 5128.816052 |
| 93.25277 | 101.372983 | 77.12163 | 118.072363 |
| 98.20619 | 112.664144 | 87.01636 | 784.165496 |
| 101.1977 | 179.003571 | 89.02475 | 3689.452008 |
| 104.2004 | 74.965155 | 89.33272 | 106.713514 |
| 110.1915 | 107.457158 | 93.0378 | 98.710896 |
| 114.1919 | 310.219885 | 94.03036 | 801.832942 |
| 124.1697 | 541.492879 | 101.0621 | 81.171167 |
| 127.1837 | 251.473709 | 112.0237 | 132.567353 |
| 135.1607 | 211.982675 | 113.0289 | 256.6648 |
| 138.1605 | 184.608718 | 121.0391 | 779.921546 |
| 143.1455 | 125.176163 | 136.0431 | 1009.934451 |
| 146.1552 | 51.686867 | 139.0477 | 321.969773 |
| 149.1498 | 146.588712 | 151.0508 | 261.80355 |
| 151.1432 | 124.434696 | 155.0082 | 440.154667 |
| 152.1561 | 426.709823 | 157.0101 | 177.738929 |
| 159.1281 | 92.057677 | 165.0284 | 587.801494 |
| 163.1568 | 508.251143 | 171.107 | 197.524616 |
| 164.1678 | 51.884042 | 176.0796 | 211.346721 |
| 166.156 | 235.18718 | 186.0511 | 116.599949 |
| 168.1337 | 78.968582 | 187.0408 | 1166.858983 |
| 169.1348 | 260.595417 | 188.0499 | 158.886766 |
| 171.1442 | 59.12023 | 203.044 | 112.787336 |
| 173.1572 | 113.644235 | 205.13 | 132.109702 |
| 176.1467 | 108.331449 | 207.1191 | 131.606635 |
| 179.1507 | 137.84007 | 215.0732 | 5416.379733 |
| 180.1665 | 994.055767 | 215.4763 | 298.566964 |
| 185.1359 | 150.707896 | 216.0802 | 729.308918 |
| 186.1501 | 158.322059 | 217.0876 | 99.231184 |
| 190.1563 | 183.096859 | 221.1137 | 111.97474 |
| 195.175 | 86.546205 | 228.0888 | 100.697547 |
| 199.1673 | 227.035116 | 230.0733 | 604.711842 |
| 204.1508 | 71.482813 | 231.0731 | 1097.598023 |
| 205.156 | 282.427685 | 232.0814 | 111.295636 |
| 207.1617 | 187.039509 | 234.1212 | 267.144226 |
| 209.1427 | 76.891885 | 235.1485 | 202.94284 |
| 212.1917 | 121.104614 | 247.0839 | 111.958677 |
| 217.1726 | 778.327585 | 347.5377 | 55.470255 |
| 218.1681 | 219.204541 | 353.1065 | 49.397762 |
| 222.1693 | 68.666762 | 374.1498 | 462.267476 |
| 223.091 | 736.949569 | 381.5363 | 65.488965 |
| 225.161 | 83.791408 | | |
| 227.1636 | 179.282801 | | |
| 234.1969 | 351.374295 | | |
| 235.1968 | 221.299761 | | |
| 237.171 | 165.239214 | | |
| 244.1914 | 173.437145 | | |
| 253.1741 | 170.977467 | | |
| 255.2016 | 151.941156 | | |
| 257.2369 | 211.908424 | | |
| 269.2121 | 633.77052 | | |
| 270.2101 | 154.111628 | | |
| 271.2321 | 1124.577818 | | |
| 272.2465 | 339.732994 | | |
| 273.2465 | 1044.173233 | | |
| 274.2533 | 215.595509 | | |
| 279.1588 | 1902.133282 | | |
| 280.1583 | 320.860255 | | |
| 281.2123 | 96.009582 | | |
| 283.2191 | 1281.201573 | | |
| 284.2204 | 240.818719 | | |
| 285.2101 | 533.098708 | | |
| 286.2286 | 253.564416 | | |
| 287.2236 | 1550.802257 | | |
| 288.2426 | 3224.93612 | | |
| 289.2434 | 3384.734363 | | |
| 290.2552 | 810.746561 | | |
| 291.2548 | 287.438135 | | |
| 293.2133 | 105.940041 | | |
| 295.2189 | 297.089805 | | |
| 297.2621 | 150.897354 | | |
| 298.2583 | 58.674498 | | |
| 299.2333 | 353.940052 | | |
| 300.28 | 277.224355 | | |
| 301.2168 | 662.198609 | | |
| 302.2438 | 316.351463 | | |
| 303.2279 | 1157.364552 | | |
| 304.2443 | 2292.402403 | | |
| 305.2408 | 3391.780079 | | |
| 305.5312 | 171.674883 | | |
| 306.2432 | 871.724411 | | |
| 307.2501 | 5097.759878 | | |
| 307.5592 | 135.272649 | | |
| 307.8653 | 67.055551 | | |
| 308.2576 | 1307.87184 | | |
| 309.2461 | 276.320258 | | |
| 314.2569 | 196.483658 | | |
| 315.2256 | 218.14155 | | |
| 316.2783 | 914.795178 | | |
| 317.2599 | 331.764991 | | |
| 318.2375 | 59.32597 | | |
| 319.2205 | 718.902252 | | |
| 320.2407 | 260.17705 | | |
| 321.235 | 2454.356967 | | |
| 322.2501 | 822.288344 | | |
| 323.2512 | 2417.876001 | | |
| 324.264 | 599.186884 | | |
| 325.2689 | 204.666646 | | |
| 331.2688 | 147.777759 | | |
| 335.2215 | 345.293408 | | |
| 336.2407 | 147.720225 | | |
| 337.2279 | 1077.500668 | | |
| 338.2533 | 412.261973 | | |
| 339.2448 | 1476.416047 | | |
| 340.2592 | 514.704806 | | |
| 344.3092 | 193.613385 | | |
| 345.227 | 60.106943 | | |
| 347.2457 | 2194.894335 | | |
| 348.2636 | 711.622206 | | |
| 349.2606 | 4190.740285 | | |
| 350.262 | 988.545178 | | |
| 351.2579 | 404.799383 | | |
| 353.2215 | 300.675765 | | |
| 354.2486 | 152.247089 | | |
| 355.2466 | 416.642895 | | |
| 356.259 | 552.671805 | | |
| 357.2717 | 201.754991 | | |
| 361.2327 | 90.263863 | | |
| 363.2422 | 1061.838748 | | |
| 364.2584 | 266.66125 | | |
| 365.2561 | 1352.426638 | | |

The cinnamon polysaccharide yield was 1.3% by mass weight based on the original cinnamon bark feedstock. The purity of the polysaccharide fraction was 290-470 mg/g dextran standard equivalent indicating a purity of >95% cinnamon polysaccharide chemical constituents in the fraction. Comparing the analysis of total phenolic acids in solution before and after the precipitation, the precipitation appeared to have no effect on the phenolic acids. Based on a large number and variety of experimental approaches, it is quite reasonable to conclude that 1.3% yield is almost 100% of the water soluble-ethanol insoluble polysaccharides in the natural cinnamon species feedstock material.

### Example 4

Example of Step 3: Hydroalcoholic Leaching Extraction

A typical example of a 3 stage solvent extraction of the phenolic acid chemical constituents of cinnamon species is as follows: The feedstock was 2 gm of ground cinnamon bark SFE residue from Step 1 SCCO₂ (40° C., 300 bar) extraction of the essential oil. The solvent was 40 ml of 25% aqueous ethanol. In this method, the feedstock material and 40 ml aqueous ethanol were separately loaded into 100 ml extraction vessel and mixed in a heated water bath at 40° C. for 4 hours. The extraction solution was filtered using Fisherbrand P4 filter paper having a particle retention size of 4-8 [symbol] m, centrifuged at 2000 rpm for 20 minutes, and the particulate residue used for further extraction. The filtrate (supernatant) was collected for yield calculation and HPLC analysis. The residue of Stage 1 was extracted for 2 hours (Stage 2) and the residue from Stage 2 was extracted for 2 hours using the aforementioned methods. The supernatants were collected for mass balance, HPLC analysis for cinnamaldehyde (CND), catechin (C), and epicatechin (EC) in the extracts. Folin-Ciocalteu assay was used for measuring total phenolic acid concentration (purity) and protein precipitation method was used for measuring tannin acid purity. The results are shown in Table 16.

TABLE 16
Effect of multiple hydroalcoholic leaching stages on extraction yield

| | | Purity (%) | | | | | Yield (%) | | | |
|---|---|---|---|---|---|---|---|---|---|---|
| Stage | Yield (%) | CND | C | EC | TPA | TA | CND | C | EC | TPA |
| 1 | 11.05 | 4.66 | 2.33 | 15.75 | 63.26 | 14.8 | 0.52 | 0.26 | 1.74 | 6.99 |
| 2 | 6.56 | 8.20 | 3.00 | 18.42 | 65.39 | 23.1 | 0.54 | 0.20 | 1.21 | 4.29 |
| 3 | 0.41 | 5.73 | 2.98 | 16.51 | 51.44 | 81.7 | 0.02 | 0.01 | 0.07 | 0.21 |

Note:
1. CND = trans-cinnalmaldehyde; C = (+)-catechin; EC = (−)-epicatechin; TPA = total phenolic acid; TA = tannin acid.
2. CND, C, EC were analyzed by HPLC; TPA was analyzed by Folin-Ciocalteu method by using Gallic acid as standard; TA was analyzed by protein-precipitation method.

In order to verify Folin-Ciocalteu method, known phenolics acid, kaempherol, caffeic acid, catechin, at concentration of 1 mg/ml were tested. The experimental error measuring kaempherol and catechin was in the order of 2-4% and that in caffeic acid case was about 10%. In addition, one reference (Sindhu 2006) tested total phenol acid in their method extracts and the results was 289_{—}2.2 mg gallic acid/g extracts, which is fairly close to the present results.

### Example 5

Example of Step 4 Affinity Adsorbent Extraction of Purified Polyphenolic Acid Fraction

In typical experiments, the working solution was the transparent hydroalcoholic solution of cinnamon species aqueous ethanol leaching extract in Step 3. The affinity adsorbent polymer resin was Sephadex LH-20. 6 gm of affinity adsorbent was pre-washed with 95% ethanol (4-5 BV) before packing into a column with an ID of 1.5 cm and length of 100 cm. The packed column volume was 25 ml. 100 ml of cinnamon 25% ethanol stage I+stage II extraction solution (sample solution. 2.4 mg/ml) was concentrated to 1 ml using rotary evaporation to remove the solvent. Then, 19 ml of absolute ethanol was added to the concentrated solution to dissolve the chemical constituents. This solution was centrifuged at 2000 rpm for 10 minutes and the supernatant collected as the final polyphenolic loading solution (11 mg/ml). 12 ml of the loading solution was loaded onto the column. The loaded column was eluted with 240 ml of 95% ethanol at a flow rate of 2.4 BV/hr (1 ml/min) with an elution time of 100 minutes. During elution, 8 non-tannin polyphenolic fractions were collected (labeled Elution Fraction F1-F8) at each 30 ml of elution. Each fraction was tested using UV spectrophotometry at 280 nm until the absorbance could no longer be detected in the fraction collected. The column washed with 70 ml of 70% aqueous acetone to remove the tannin polyphenolics adsorbed on the affinity adsorbent at a flow rate of 5 BV/hr (2.1 ml/min). The tannin washing solution was discarded. Finally, the column washed with 4-5 BV of 95% ethanol to remove any remaining chemical impurities in order to prepare the column for further processing. Each polyphenolic elution fraction was collected and analyzed and the results are shown in Table 17.

TABLE 17
Analysis of 95% ethanol elutions of polyphenolic fractions from Sephadex LH-20
process chromatography.

| | | Weight (mg) | | | | Purity (%) | | |
|---|---|---|---|---|---|---|---|---|
| | | | Total | | | Non | | |
| | Yield | Total | phenolic | Tannin | Nontannin | Tannin | tannin | Average |
| Name | (%) | solid | acid | acid | acid | acid | acid | DPn |
| Loading | | 132.1 | 61.2 | 32.8 | 28.5 | 21.6 | 24.8 | 6.9 |
| Elution F2 | 37.1 | 49.0 | 3.7 | 0.1 | 3.6 | 7.1 | 0.1 | 3.6 |
| Elution F3 | 7.4 | 9.8 | 2.9 | 0.0 | 2.9 | 29.5 | 0.0 | 2.7 |
| Elution F4 | 5.2 | 6.8 | 4.5 | 0.0 | 4.5 | 66.4 | 0.0 | 3.6 |
| Elution F5 | 3.2 | 4.2 | 3.7 | 0.0 | 3.7 | 87.8 | 0.0 | 3.1 |
| Elution F6 | 2.3 | 3.1 | 2.9 | 0.0 | 2.9 | 91.1 | 0.0 | 4.0 |
| Elution F7 | 2.1 | 2.8 | 2.9 | 0.0 | 2.9 | 100.0 | 0.0 | 4.2 |
| Elution F8 | 1.2 | 1.6 | 1.6 | 0.0 | 1.6 | 93.8 | 0.0 | 4.2 |
| Combine | 5.7 | 7.5 | 7.3 | 0.0 | 7.3 | 97.2 | 0.0 | 4.1 + 0.1 |
| F6-F8 | | | | | | | | |
| Combine | 21.5 | 28.4 | 18.5 | 0.0 | 18.5 | 65.1 | 0.0 | 3.6 ⊥ 0.6 |
| F2-F8 | | | | | | | | |
| Recovery | | 58.5 | 36.1 | 0.2 | 77.4 | | | |
| (%) | | | | | | | | |

* Elution 1 was not tabulated because there were no chemical constituents, only solvent.

### Example 6

The following ingredients are mixed for the formulation:

| | |
|---|---|
| Extract of C. cassia bark | 150.0 mg |
| Essential Oil Fraction (10 mg, 6.6% dry weight) | |
| Polyphenolic Fraction (100 mg, 66.7% dry weight) | |
| Polysaccharides (40 mg, 26.6% dry weight) | |
| Stevioside (Extract of Stevia) | 12.5 mg |
| Carboxymethylcellulose | 35.5 mg |
| Lactose | 77.0 mg |
| Total | 275.0 mg |

The novel extract of cinnamon species comprises an essential oil fraction, phenolic acid-essential oil fraction, and polysaccharide fraction by % mass weight greater than that found in the natural rhizome material or convention extraction products. The formulations can be made into any oral dosage form and administered daily or to 15 times per day as needed for the physiological and psychological effects desired (enhanced brain function and analgesia) and medical effects (non-insulin dependent diabetes mellitus, anti-platelet aggregation and anti-thrombosis, cardiovascular and cerebrovascular disease prevention and treatment, anti-atherosclerosis, anti-hypercholesterolemia, cardiac protection, nervous system protection, anti-inflammatory, anti-allergic, anti-arthritis, anti-rheumatic, anti-gout, gastro-intestinal disorders, cough, common cold, fever, lipolytic, improved wound healing, anti-bacterial, anti-fungal, and anti-cancer).

### Example 7

The following ingredients were mixed for the following formulation:

| | |
|---|---|
| Extract of C. cassia | 150.0 mg |
| Essential Oil Fraction (60 mg, 40% dry weight) | |
| Polyphenolic Fraction (30 mg, 20% dry weight) | |
| Polysaccharides (60.0 mg, 40% dry weight) | |
| Vitamin C | 15.0 mg |
| Sucralose | 35.0 mg |
| Mung Bean Powder 10:1 | 50.0 mg |
| Mocha Flavor | 40.0 mg |
| Chocolate Flavor | 10.0 mg |
| Total | 300.0 mg |

The novel extract of cinnamon chuangxiong comprises an essential oil, phenolic acid-essential oil, and polysaccharide chemical constituent fractions by % mass weight greater than that found in the natural plant material or conventional extraction products. The formulation can be made into any oral dosage form and administered safely up to 15 times per day as needed for the physiological, psychological and medical effects desired (see Example 1, above).

### REFERENCES

1. Khan A et al. Diabetes Care 26:3215-3218, 2003.
2. Anderson R A et al. J Agric Food Chem 52:65-70, 2004.
3. Jarville-Taylor et al. J Am Coll Nutri 20:327-336, 2001.
4. Qin R et al. Horm Metab Res 36:119-123, 2004.
5. Vespohl E J et al. Phytother Res 19:203-206, 2005.
6. Lee S H et al Biochem Pharmacol 69:791-9, 2005.
7. Chericoni S et al. J Agric Food Chem 53:4762-4765, 2005.
8. Lin C C et al. Phytother Res 17: 7260730,2003.
9. Jayaprakasha G K et al. J Agric Food Chem 51:4344-4348, 2003.
10. Huss U et al. J Nat Prod 65:1517-21, 2002.
11. Nagai H et al. Jpn J Pharmacol 32:813-822, 1982.
12. Su M J et al. J Biomed Sci 6:376-386, 1999.
13. Shimada Y et al. Phytomed 11:404-410, 2004.
14. Taher M et al. Med J Malayia 59B:97-98, 2004.
15. Kamath J V et al. Phytother Res 17:970-972, 2003.
16. Kurokawa M et al. Eur J Pharmacol 348:45-51, 1998.
17. Simic A et al. Phytother Res 18:713-717, 2004.
18. Tabak M et al. J Ethnopharmacol 67:269-277, 1999.
19. Kong L D et al. J Ethnopharmacol 73:199-207, 2000.
20. Kwon B M et al. Arch Pharm Res 21:147-152, 1998.
21. Ka H et al. Cancer Lett 196:143-152, 2003.
22. Williamson E M. Phtomedicine 8:401-409, 2001.
23. Dubois M et al. Analytical Chem 28:350-356, 1956.
24. Gu L et al. J Agric Food Chem 51:7513-7521, 2003.
25. Guyot S et al. Methods in Enzymology 335:57-70, 2001.
26. Maria Jerez P et al. Food Chem 94:406-414, 2006.
27. Makkar H P S et al. J Sci Food Agric 61:161-165, 1993.
28. Makkar H P S et al. J Agric Food Chem 36:523-525, 1988.]
29. Shindu, M. Food Chem 94:520-528, 2006.

We claim:

1. A cinnamon species extract comprising a fraction having a Direct Analysis in Real Time (DART) mass spectrometry chromatogram of any of FIGS. 6 to 85.

2. The cinnamon species extract of claim 1, wherein the fraction comprises a compound selected from the group consisting of cinnamaldehyde, benzaldehyde, cinnamyl alcohol, trans-cinnamic acid, cinnamyl acetate, an essential oil, a polyphenol, a polysaccharide, and combinations thereof.

3. The cinnamon species extract of claim 2, wherein the fraction comprises cinnamaldehyde in an amount greater than about 2% by weight.

4. The cinnamon species extract of claim 2, wherein the fraction comprises cinnamaldehyde in an amount greater than about 5, 10, 15, 20, 25, 30, 35, 40, 45, 50, 55, 60, 65, 70, 75, 80, 85, 90, or 95% by weight.

5. The cinnamon species extract of claim 2, wherein the fraction comprises cinnamaldehyde in an amount from about 65% to about 95% by weight.

6. The cinnamon species extract of claim 2, wherein the fraction comprises an essential oil selected from the group consisting of eugenol, 2′-hydroxycinnamaldehyde, 2-methoxycinnamaldehyde, 2′-benzoxycinnamaldehyde, linalool, 1,8-cineole, alpha-pinene, beta-pinene, and combinations thereof.

7. The cinnamon species extract of claim 2, wherein the fraction comprises essential oil in an amount from about 1% to about 5% by weight.

8. The cinnamon species extract of claim 2, wherein the fraction comprises a combined amount of cinnamaldehyde and essential oil of about 5% to about 40% by weight.

9. The cinnamon species extract of claim 2, wherein the fraction comprises a polyphenol selected from the group consisting of flavonoid, flavonol glycoside, and combinations thereof.

10. The cinnamon species extract of claim 2, wherein the fraction comprises a polyphenol in an amount from about 20% to about 70% by weight.

11. The cinnamon species extract of claim 2, wherein the fraction comprises cinnamaldehyde at about 6% by weight and a polyphenol at about 70% by weight.

12. The cinnamon species extract of claim 2, wherein the fraction comprises cinnamaldehyde at about 40% by weight and a polyphenol at about 20% by weight.

13. The cinnamon species extract of claim 2, wherein the fraction comprises a polysaccharide selected from the group consisting of glucose, arabinose, galactose, rhamnose, xylose uronic acid and combinations thereof.

14. The cinnamon species extract of claim 2, wherein the fraction comprises a polysaccharide at about 30% by weight.

15. The cinnamon species extract of claim 9, wherein the flavonoid is selected from the group consisting of 3-(2-hydroxyphenyl)-propanoic acid, 3-(2-hydroxyphenyl)-O-glycoside, anthocyanidin, epitcatechin, catechin, methylhydroxychalcone, catechin oligomers, epicatechin oligomers, oligomeric proanthocyanidins, polymeric proanthocyanidins, and combinations thereof.

16. The cinnamon species extract of claim 9, wherein the flavonol glycoside is selected from the group consisting of kaempferitrin, kaempferol 3-O-Beta-D-glucopyranosyl-(1→4)-alpha-L-rhamnopyranoside, kaempferol 3-O-beta-D-apiofuranosyl-(1→42)-alpha-L-rhamnopyranoside, kaempferol 3-O-beta-D-apiofuranosyl-(1→4)-alpha-L-rhamnopyranoside, and combinations thereof.

17. Food or medicament comprising the cinnamon species extract of claim 1.

18. A method of preparing a cinnamon extract comprising sequentially extracting a cinnamon species plant material to yield an essential oil fraction, a non-tannin polyphenolic fraction and a polysaccharide fraction by
a) extracting cinnamon species plant material by supercritical carbon dioxide extraction to yield the essential oil fraction and a first residue;
b) extracting cinnamon species plant material or the first residue from step a) by water at about 70° C. to about 90° C. extraction and precipitating the polysaccharide with alcohol to yield the polysaccharide fraction and a second residue; and
c) extracting cinnamon species plant material, the first residue from step a) and/or the second residue from step b) with a hydro-alcoholic solution and purifying the extraction using affinity adsorbent processes to yield the non-tannin polyphenolic fraction.

19. The method of claim 18, wherein step a) comprises
1) loading in an extraction vessel ground cinnamon species plant material;
2) adding carbon dioxide under supercritical conditions;
3) contacting the ground cinnamon bark and the carbon dioxide for a time; and
4) collecting an essential oil fraction in a collection vessel.

20. The method of claim 19, wherein supercritical conditions comprise 60 bars to 800 bars of pressure at 35° C. to 90° C.

21. The method of claim 19, wherein supercritical conditions comprise 60 bars to 500 bars of pressure at 40° C. to 80° C.

22. The method of claim 19, wherein the time is 30 minutes to 2.5 hours.

23. The method of claim 19, wherein the time is 1 hour.

24. The method of claim 19, wherein a supercritical carbon dioxide fractional separation system is used for fractionation, purification, and profiling of the essential oil fraction.

25. The method of claim 18, wherein step b) comprises
1) contacting ground cinnamon species plant material or the first residue from step a) with a water for a time sufficient to extract polysaccharide chemical constituent; and
2) separating and purifying the solid polysaccharides from the solution by alcohol precipitation.

26. The method of claim 25, wherein the water is at 70° C. to 90° C.

27. The method of claim 25, wherein the water is at 80° C. to 90° C.

28. The method of claim 25, wherein the time is 1-5 hours.

29. The method of claim 25, wherein the time is 2-4 hours.

30. The method of claim 25, wherein the time is 2 hours.

31. The method of claim 25, wherein the alcohol is ethanol.

32. The method of claim 18, wherein step c) comprises:
1) contacting cinnamon species plant material, the first residue from step a) and/or the second residue from step b) with hydroalcoholic solution for a time sufficient to extract polyphenolic chemical constituents;
2) passing a concentrated alcohol solution of extracted polyphenolic chemical constituents from the hydroalcoholic solvent mixture through an affinity adsorbent resin column wherein the polyphenolic acids are adsorbed; and
3) eluting the purified non-tannin polyphenolic chemical constituent fraction(s) from the affinity adsorbent resin leaving the tannin polyphenolics adsorbed to the affinity adsorbent resin.

33. The method of claim 32, wherein the hydroalcoholic solution comprises ethanol and water wherein the ethanol concentration is 10-95% by weight.

34. The method of claim 32, wherein the hydroalcoholic solution comprises ethanol and water wherein the ethanol concentration is 25% by weight.

35. The method of claim 32, wherein step 1) is carried out at 30° C. to 100° C.

36. The method of claim 32, wherein step 1) is carried out at 60° C. to 100° C.

37. The method of claim 32, wherein the time is 1-10 hours.

38. The method of claim 32, wherein the time is 1-5 hours.

39. The method of claim 32, wherein the time is 2 hours.

40. A cinnamon species extract prepared by the method of claim 18.

41. A cinnamon species extract comprising cinnamaldehyde, cinnamic acid at 1 to 5% by weight of the cinnamaldehyde, methyl cinnamic acid at 5 to 15% by weight of the cinnamaldehyde, cinnamyl alcohol at 1 to 5% by weight of the cinnamaldehyde, β-gualenen/cis-γ-bisababolene at 20 to 30% by weight of the cinnamaldehyde, and pyrogallol at 1 to 5% by weight of the cinnamaldehyde.

42. A cinnamon species extract comprising pyrogallol, cinnamic acid at 80 to 90% by weight of the pyrogallol, methyl cinnamic acid at 85 to 95% by weight of the pyrogallol, coumaric acid at 20 to 30% by weight of the pyrogallol, homovanillic acid at 15 to 25% by weight of the pyrogallol, cinnamaldehyde at 85 to 95% by weight of the pyrogallol, and benzyl benzoate at 10 to 15% by weight of the pyrogallol.

43. A cinnamon species extract comprising catechin, cinnamic acid at 5 to 15% by weight of the catechin, methyl cinnamic acid at 5 to 15% by weight of the catechin, coumaric acid at 5 to 15% by weight of the catechin, ferulic acid at 1 to 10% by weight of the catechin, 2-methoxyphenol at 1 to 5% by weight of the catechin, homovanillic acid at 5 to 15% by weight of the catechin, vanillic acid at 20 to 30% by weight of the catechin, benzaldehyde at 1 to 5% by weight of the catechin, cinnamaldehyde at 35 to 45% by weight of the catechin, pyrogallol at 85 to 95% by weight of the catechin, and caffeic acid at to 15% by weight of the catechin.

44. A cinnamon species extract comprising β-gualenen/cis-γ-bisababolene and cinnamaldehyde at 5 to 15% by weight of the β-gualenen/cis-γ-bisababolene.

45. A cinnamon species extract comprising cinnamaldehyde and β-gualenen/cis-γ-bisababolene at 10 to 20% by weight of cinnamaldehyde.

46. A cinnamon species extract comprising cinnamaldehyde, pyrogallol at 30 to 40% by weight of the cinnamaldehyde, and catechin/epicatechin at 1 to 10% by weight of cinnamaldehyde.

47. A cinnamon species extract comprising cinnamaldehyde, cinnamic acid at 1 to 5% by weight of the cinnamaldehyde, methoxy cinnamaldehyde at 0.5 to 5% by weight of the cinnamaldehyde, eugenol at 0.1 to 5% by weight of the cinnamaldehyde, p-cymene at 1 to 5% by weight of the cinnamaldehyde, camphor at 0.1 to 5% by weight of the cinnamaldehyde, carvacrol at 0.5 to 5% by weight of the cinnamaldehyde, caryophyllene/humulene at 25 to 35% by weight of the cinnamaldehyde, pyrogallol at 0.1 to 5% of the cinnamaldehyde, and cinnamyl cinnamate at 40 to 50% by weight of the cinnamaldehyde.

48. A cinnamon species extract comprising cinnamyl cinnamate, methoxy cinnamaldehyde at 0.5 to 5% by weight of the cinnamyl cinnamate, cinnamyl alcohol at 0.1 to 5% by weight of the cinnamyl cinnamate, p-cymene at 1 to 5% by weight of the cinnamyl cinnamate, linalool at 0.1 to 5% by weight of the cinnamyl cinnamate, camphor at 0.1 to 5% by weight of the cinnamyl cinnamate, carvacrol at 0.5 to 5% by weight of the cinnamyl cinnamate, cinnamaldehyde at 70 to 80% by weight of the cinnamyl cinnamate, caryophyllene/humulene at 45 to 55% by weight of the cinnamyl cinnamate, and pyrogallol at 0.1 to 5% of the cinnamyl cinnamate.

49. A cinnamon species extract comprising pyrogallol, cinnamic acid at 5 to 10% by weight of the pyrogallol, coumaric acid at 60 to 70% by weight of the pyrogallol, ferulic acid at 1 to 10% of the pyrogallol, 2-methoxyphenol at 5 to 15% of the pyrogallol, vanillic acid at 1 to 10% by weight of the pyrogallol, catechin/epicatechin at 30 to 40% by weight of the pyrogallol, benzaldehyde at 1 to 5% by weight of the pyrogallol, afzelechin/epiafzelechin at 5 to 15% by weight of the pyrogallol, resveratrol at 1 to 10% by weight of the pyrogallol, and vanillin at 1 to 5% by weight of the pyrogallol.

50. A cinnamon species extract comprising pyrogallol, cinnamic acid at 0.5 to 5% by weight of the pyrogallol, coumaric acid at 10 to 20% by weight of the pyrogallol, ferulic acid at 0.5 to 5% of the pyrogallol, 2-methoxyphenol at 1 to 5% of the pyrogallol, homo/isovanillic acid at 0.5 to 5% by weight of the pyrogallol, vanillic acid at 1 to 10% by weight of the pyrogallol, catechin/epicatechin at 25 to 35% by weight of the pyrogallol, benzaldehyde at 1 to 5% by weight of the pyrogallol, cinnamaldehyde at 1 to 5% of the pyrogallol, afzelechin/epiafzelechin at 0.1 to 5% by weight of the pyrogallol, and vanillin at 65 to 75% by weight of the pyrogallol.
